SEQUENCE LISTING (5 sequences)

SEQ ID NO: 1 - DNA, 66 bp, Artificial sequence
  Synthetic dsDNA oligonucleotide
ttaagaatttaatacgactcactatagggagagtgactggagttcagacgtgtgctcttccgatct

SEQ ID NO: 2 - DNA, 70 bp, Artificial sequence
  Synthetic ds DNA oligonucleotide
cgaaagatcggaagagcacacgtctgaactccagtcactctccctatagtgagtcgtattaaattcttaa

SEQ ID NO: 3 - DNA, 23 bp, Artificial sequence
  Synthetic dsDNA oligonucleotide
caacgtgattgcttgtgacttaa

SEQ ID NO: 4 - DNA, 19 bp, Artificial sequence
  Synthetic dsDNA oligonucleotide
ttaagtcacaagcaatcac

SEQ ID NO: 5 - DNA, 27 bp, Artificial sequence
  Synthetic primer
  misc_feature (22)..(27): n is any nucleotides
  misc_feature (22)..(27)
tacacgacgctcttccgatctnnnnnn

(19) United States
(12) Patent Application Publication
(10) Pub. No.: US 2022/0206003 A1
(43) Pub. Date: Jun. 30, 2022

(54) BIOMARKERS FOR PREDICTING RESISTANCE TO CANCER DRUGS

(71) Applicants: INSTITUT CURIE, Paris (FR); HIFIBIO, Paris (FR); CENTRE NATIONAL DE LA RECHERCHE SCIENTIFIQUE - CNRS, Paris (FR); SORBONNE UNIVERSITE, Paris (FR); ECOLE SUPERIEURE DE PHYSIQUE ET DE CHIMIE INDUSTRIELLE DE LA VILLE DE PARIS, Paris (FR); PARIS SCIENCES ET LETTERS - QUARTIER LATIN, Paris (FR)

(72) Inventors: Annabelle GERARD, Palaiseau (FR); Kevin GROSSELIN, Paris (FR); Andrew GRIFFITHS, Paris (FR); Céline VALLOT, Paris (FR)

(21) Appl. No.: 17/605,730

(22) PCT Filed: Apr. 24, 2020
(86) PCT No.: PCT/EP2020/061459

§ 371 Date: Oct. 22, 2021

(30) Foreign Application Priority Data
Apr. 24, 2019 (EP) 19305527.4

(51) Int. Cl. G01N 33/574

(52) U.S. Cl. CPC G01N 33/57415; G01N 33/57484

(57) ABSTRACT

The present invention relates to biomarkers which comprise one or more genomic sequence(s) comprising epigenetic modification and their use in a method for predicting resistance to cancer treatment, in particular for patient stratification.

### FIELD OF THE INVENTION

The present invention relates to biomarkers which comprise one or more genomic sequence(s) comprising epigenetic modification and their use in a method for predicting resistance to or assessing possible outcomes of cancer treatment, in particular for patient stratification.

### BACKGROUND OF THE INVENTION

The emergence of resistance to chemotherapy and targeted therapies is a major challenge for the treatment of cancer. Genetic heterogeneity within untreated tumors is now considered to be a key determinant of resistance; sub-population of cells bearing a mutation conveying resistance can survive and be selected in a Darwinian process (Schmitt, M. W. et al. 2016. Nat Rev Clin Oncol 13, 335-347). Deep sequencing and single-cell approaches have revealed the importance of genetic intra-tumor heterogeneity to tumor evolution (Roth, A. et al. 2016. Nat Methods 13, 573-576; Nik-Zainal, S. et al. 2012. Cell 149, 994-1007; McGranahan, N. & Swanton, C. 2017. Cell 168, 613-628) and shown that genetic heterogeneity within untreated tumors is a key factor in tumor resistance (Dagogo-Jack, I. & Shaw, A. 2018. Nat Rev Clin Oncol 15, 81-94).

However, in many cases genetic mechanisms driving resistance cannot be found, pointing to a role for non-genetic mechanisms (Salgia, R. & Kulkarni, P. 2018. Trends Cancer 4, 110-118). Non-genetic and particularly transcriptional and epigenetic mechanisms are anticipated to play a role in the adaptation of cancer cells confronted with environmental, metabolic or therapy-related stresses (Rathert, P. et al. 2015. Nature. 525, 543-547; Kim, C. et al. 2018. Cell 173, 879-893 e813).

Recent studies, using single-cell RNA sequencing (scRNA-seq), indicate that emergence of transcriptional subclones upon treatment may account for the adaptation of cancer cells to therapeutic pressure (Kim, C. et al. 2018. Cell 173, 879-893 e813; Horning, A. M. et al. 2018. Cancer Res 78, 853-864).

In contrast, only a few studies have tracked the clonal evolution of epigenetic alterations, exclusively analyzing DNA methylation at the population level (Mazor, T. et al. 2015. Cancer Cell 28, 307-317; Aryee, M. J. et al. 2013. Sci Transl Med 5, 169ra110) suggesting that DNA methylation alterations and genetic mutations shared a common evolutionary track.

Modulation of chromatin structure via histone modification is a major epigenetic mechanism and key regulator of gene expression. However, the contribution of chromatin heterogeneity to tumor evolution remains unknown.

Profiling histone modifications at single-cell resolution remains challenging, in part because the level of noise associated with non-specific binding during the immunoprecipitation tends to increase with low quantity of starting material. Immunoprecipitating chromatin from one single cell is not technically feasible and one therefore needs to tag the chromatin of each single-cell prior to immuno-precipitation, before pooling the chromatin fragments of several thousand cells and performing immuno-precipitation.

Until now, insufficient coverage has limited the applications of single-cell chromatin profiling to cell lines (Rotem, A. et al. 2015. Nat Biotechnol 33, 1165-1172; WO 2013/134261), preventing the study of the heterogeneity of chromatin states in complex biological systems such as tumors.

### SUMMARY

Using an improved single-cell ChIP method based on droplet microfluidics to profile chromatin landscapes of thousands of cells at single-cell resolution with a coverage of 10,000 loci/cell, the inventors detected the presence of relatively rare chromatin states within tumor samples. In patient-derived xenografts models of acquired resistance to chemotherapy and targeted therapy in breast cancer, the inventors found that respectively 3% and 16% of cells in the untreated tumors possessed chromatin features characteristic of resistant cancer cells. These cells, and cells from the resistant tumors, had lost chromatin marks (H3K27me3) on specific genomic sequences. Some of which are associated with stable transcriptional repression for genes known to promote resistance to treatment, potentially priming them for transcriptional activation.

In certain embodiments, the present invention relates to a biomarker for determining resistance to treatment of a cancer type with a cancer drug which comprises one or more genomic sequence(s) comprising a histone modification, wherein said one or more genomic sequence(s) is selected from the list of Tables 1 to 6. In particular, said histone is in a gene or in the proximity of said gene and said genomic sequences is selected from the list of Table 1 and 4. Preferably, the invention relates to a biomarker as described above for predicting resistance to treatment of a cancer type with a cancer drug prior administration of a treatment to a patient. In another aspect, the invention provides a method for determining the resistance to treatment of a cancer type with a cancer drug to a patient comprising: i) detecting a histone modification of at least one biomarker as described above which comprises one or more genomic sequence(s) selected from the list of Tables 1 to 6 in said patient tumor sample, and ii) determining from the presence or absence of histone modification of said biomarker, whether the patient is likely to be resistant or sensitive to said treatment. In certain embodiments, the present invention also provides a method for determining the resistance to treatment of a cancer type with a cancer drug to a patient comprising: i) determining in said patient tumor sample the expression level of a gene encoding by a biomarker as described above which comprises one or more genomic sequence(s) selected from the list of Tables 1 and 4, ii) determining from the expression level of said gene whether a patient is likely to be resistant or sensitive to said treatment.

Preferably, said method is realized prior to administration of any treatment or said treatment to the patient. Particularly, said cancer drug agent is a chemotherapy drug, preferably capecitabine and said genomic sequence is selected from the list of Tables 1 to 3. In another aspect, said cancer drug agent is an anti-hormonal drug, preferably tamoxifen and said genomic sequence is selected from the list of Tables 4 to 6.

Preferably, said histone modification is associated with transcriptional activation, more preferably said histone modification is a loss of transcriptional repressive chromatin marks, in particular H3K27me3, in said genomic sequence. Cancer according to the disclosure is preferably a breast cancer, preferably triple negative breast cancer.

In another aspect, the invention provides a combined preparation comprising a cancer drug and a compound that modulates the epigenetic status of the biomarker as described above, for use in cancer treatment, to reduce the development of resistance to said cancer treatment. Said combined preparation may comprise a cancer drug and a compound that modulates the epigenetic status of the biomarker as described above, for use in cancer treatment to reduce the development of resistance to said cancer treatment wherein said compound is administered before, after or concurrently with the therapeutic drug.

### DETAILED DESCRIPTION OF THE INVENTION

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

“Treating cancer” includes, without limitation, reducing the number of cancer cells or the size of a tumor in the patient, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms or slowing the progression of symptoms, etc.

As used herein, “drug” or “therapeutic agent” refers to a compound or agent that provides a desired biological or pharmacological effect when administered to a human or animal, particularly results in an intended therapeutic effect or response on the body to treat or prevent conditions or diseases. Therapeutic agents include any suitable biologically-active chemical compounds, biologically derived components such as for example small molecules, cells, proteins, peptides, antibodies, enzymes, polynucleotides, and radiochemical therapeutic agents, such as radioisotopes.

As used herein, a “therapeutic response” or “response to treatment with a drug” refers to a positive medical response characterized by objective parameters or criteria such as objective clinical signs of the disease, patient self-reported parameters and/or the increase of survival. The objective criteria for evaluating the response to drug-treatment will vary from one disease to another and can be determined easily by one skilled in the art by using clinical scores. A positive medical response to a drug can be readily verified in appropriate animal models of the disease which are well-known in the art and illustrated in the examples of the present application.

The term “determining resistance to a treatment with a drug”, as used herein, refers to an ability to assess whether the treatment of a patient with a drug will stop being effective in (e.g., stop providing a measurable benefit or positive medical response to) the subject after some time of administration of the treatment. In other terms, determining the resistance to a treatment refers to an ability to assess possible outcome of treatment of a cancer type. The resistance of a patient to a therapeutic agent may be determined by the lack of improvement in the disease state as measured by the absence of positive medical response, as compared to pre-treatment. In particular, such an ability to assess whether the treatment will stop being effective typically is exercised before treatment with the drug has begun in the subject.

However, it is also possible that such an ability to assess whether the treatment will stop being effective can be exercised after treatment has begun but before an indicator of ineffectiveness has been observed in the patient.

The term “predicting resistance to a treatment with a drug”, as used herein, refers to an ability to assess whether the treatment of a patient with a drug will stop being effective in (e.g., stop providing a measurable benefit or positive medical response to) the subject before treatment with the drug has begun in the subject.

The terms “subject” and “patient” are used interchangeably herein and refer to both human and non-human animals. As used herein, the term “patient” denotes a mammal, such as a rodent, a feline, a canine, and a primate. Preferably, a patient according to the invention is a human.

“a”, “an”, and “the” include plural referents, unless the context clearly indicates otherwise. As such, the term “a” (or “an”), “one or more” or “at least one” can be used interchangeably herein.

Biomarkers for Determining the Cancer Treatment Resistance

The inventors by using improved single-cell chromatin profiling identified epigenetic modifications in a specific genomic sequence characteristic of drug-resistant tumor cells.

These epigenetic modified genomic sequences can be used as biomarkers for determining drug-resistance in cancer patients, preferably before administration of cancer treatment and are listed in Tables 1 to 6. The genomic sequences listed in Tables 1 to 6 are identified by an identification number of the chromosomic region using the reference genome GRCh38 (Genome Reference Consortium Human Build 38 submitted in Dec. 17, 2013) (hg38) (GenBank assembly accession: GCA_000001405.15).

Certain embodiments of the present invention relate to a biomarker for determining resistance to treatment of a cancer type with a cancer drug which comprises one or more genomic sequence(s) selected from the list of Table 1 to 6, more preferably Tables 1 and 4. In a preferred embodiment, said genomic sequence comprises a histone modification, in particular a loss of H3K27me3, wherein said genomic sequence is selected from the list of Tables 1 to 6. In a preferred embodiment, the present invention relates to a predictive biomarker for predicting resistance to cancer treatment with a cancer drug prior administration of a treatment to a patient.

In another embodiment, the present invention relates to an isolated biomarker for determining resistance to or assessing possible outcome of treatment of a cancer type with a cancer drug in a patient which comprises purified nucleic acids having one or more genomic sequence(s) comprising a histone modification, wherein said one or more genomic sequence(s) is selected from the list of Tables 1 to 6.

The term “biomarker” refers to a distinctive biological or biologically derived indicator of a process, event or condition.

A “predictive biomarker” as used herein refers to a biomarker that can be used in advance of therapy to estimate the resistance of a patient suffering from a particular disease to a specific treatment of said disease. The biomarker for predicting the resistance of a patient to treatment with a drug prior administration to said treatment is herein referred to as pre-treatment predictive biomarker of drug-resistance.

By “epigenetic modification” or “epigenetic information” include with no limitations: histone modification, histone variant, DNA methylation, DNA modified bases and chromatin/DNA associated factors, preferably histone modification, histone variant and chromatin/DNA associated factors, more preferably histone modification.

By “characteristic epigenetic modification” or “specific epigenetic modification” it is meant an epigenetic modification that is present in a genomic sequence of tumor cells of a drug-resistant tumor and absent in the same genomic sequence of general population or from a selected population of subjects. The general population may comprise apparently healthy subjects, such as individuals who have not previously had any sign or symptoms indicating the presence of cancer. The term “healthy subjects” as used herein refers to a population of subjects who do not suffer from any known condition, and in particular, who are not affected with any cancer. In a preferred embodiment, the selected population may comprise subjects having an established cancer but who shows a clinically significant relief in a cancer type when treated with a cancer drug as described above.

In another embodiment, the epigenetic modification is present in a specific genomic sequence of tumor cells of a drug-resistant tumor and absent in the same genomic sequence of the majority of tumor cells of an untreated sensitive tumor from which the resistant-tumor is derived. As used herein, “the corresponding tumor cells” means tumor cells of the same tumor type. For example, the corresponding sensitive tumor cells may be the untreated tumor cells from which the resistant tumor cells are derived.

Preferably, epigenetic modifications are histone modifications. Histone modifications or histone post-translational modifications may be selected from the group comprising acetylation, amidation, deamidation, carboxylation, disulfide bond, formylation, glycosylation, hydroxylation, methylation, myristoylation, nitrosylation, phosphorylation, prenylation, ribosylation, sulphation, sumoylation, ubiquitination and derivatives thereof. Said histone modifications may be associated with transcriptional activation, such as for example histone H3 lysine 4 methylation (H3K4me3, H3K4me2 or H3K4me1) and histone acetylation. Alternatively, said histone modifications may be associated with transcriptional repression, such as for example histone H3 lysine 9 trimethylation (H3K9me3), H3K27me3 and H4K20me3.

In some embodiments, said specific epigenetic modification is loss of transcriptional repressive chromatin marks, in particular H3K27me3, in said genomic sequence.

Said specific epigenetic modification may be in a gene or in the proximity of said gene, i.e. less than 1 kb from the transcription start of said gene and said biomarker comprises one or more genomic sequence(s) selected from the list of Table 1 and 4. In a preferred embodiment, said biomarker comprising an epigenetic modification in a gene or in the proximity of said gene can be useful in determining resistance of a subject to a cancer treatment by determining the histone modifications of said biomarker and/or the expression level of said gene (e.g., mRNA or protein expression levels) in a patient sample.

In another embodiment, said specific epigenetic modification is not in a gene or in the proximity of said gene, i.e. more than 1 kb from the transcription start of said gene and said biomarker comprises one or more genomic sequence(s) selected from the list of Table 2 and 5. In a preferred embodiment, said biomarker comprising an epigenetic modification which is not in a gene or in the proximity of said gene can be useful in determining resistance of a subject to a cancer type by determining the epigenetic modifications of said biomarker, preferably by single-cell epigenetic profiling using a microfluidic system as described in examples of the present application.

The use of biomarker permits to determine the resistance to treatment of any cancer type, such as solid or liquid (or blood) cancer. In some embodiments, said biomarker determines the resistance to treatment of a cancer selected from the group consisting of: breast cancer, ovarian cancer, lung carcinoma, colorectal cancer, prostate cancer, pancreatic cancer and melanoma. In some preferred embodiments, said biomarker determines the resistance to treatment of breast cancer, preferably triple-negative breast cancer.

Targeted therapy includes the use of “targeted” drugs such as small molecule inhibitors or neutralizing monoclonal antibodies, that target proteins that are abnormally expressed in cancer cells and that are essential for their growth such as for example receptor and non-receptor tyrosine kinases, growth factors, hormone receptors, and others. Preferably, said targeted drugs are anti-hormonal drugs. Examples of anti-hormonal drugs include with no limitations: Tamoxifen, targeting the estrogen receptor; The cancer agent may be a drug for chemotherapy or targeted therapy. Chemotherapy includes the use of cytotoxic anti-neoplastic agents, such as alkylating agents, anti-metabolites, anti-microtubule agents, Topoisomerase inhibitors, cytotoxic antibiotics and others. Examples of chemotherapeutic drugs include with no limitations: Capecitabine, 5-FU, docetaxel, SN-38, CPT11, cisplatin, carboplatin, etc.

In some preferred embodiment, said cancer drug is a chemotherapy drug such as Capecitabine and said biomarker comprises one or more genomic sequence(s) selected from the list of Tables 1 to 3. Epigenetic modifications in the above listed genomic sequences, in particular loss of H3K27me3 in said genes, are found in Capecitabine resistant tumors, in particular Triple-negative breast cancer tumors.

In a preferred embodiment, said epigenetic modification is in a gene or in the proximity of said gene and the biomarker which determines the resistance to chemotherapy drug such as Capecitabine comprises one or more genomic sequence(s) selected from the list of Table 1.

In another preferred embodiments, said cancer drug is an anti-hormonal drug such as Tamoxifen, targeting the oestrogen receptor and said biomarker comprises one or more genomic sequence(s) selected from the list of Tables 4 to 6. Epigenetic modifications in the above-listed genomic sequences, in particular loss of H3K27me3 in said genes, are found in Tamoxifen resistant tumors, in particular luminal ER+ breast cancer tumors.

In a preferred embodiment, said epigenetic modification is in a gene or in the proximity of said gene and the biomarker which determines the resistance to anti-hormonal drug such as Tamoxifen comprises one or more genomic sequences selected from the list of Table 4.

In a preferred embodiment, the present disclosure provides a biomarker for predicting resistance to treatment of a cancer type prior treatment administration, wherein said biomarker comprises one or more epigenetic modified genomic sequence(s) as described above.

Method for Determining the Cancer Treatment Resistance

In another aspect, the present invention relates to a method for determining the resistance to treatment of a cancer type with a cancer drug to a patient comprising detecting a histone modification in said patient tumor sample of at least one biomarker as described above which comprises one or more genomic sequence selected from the list of Table 1 to 6.

In some preferred embodiments, said specific histone modification as disclosed above is a loss of transcriptional repressive chromatin marks, in particular H3K27me3.

According to the invention, the specific epigenetic modifications present in tumor cells can be identified by any methods well-known in the art including but not limiting to ChIP-qPCR, ChIPseq, ChIP on chip, EMSA, ATACseq, FISH, immunofluorescence, immuno-histochemistry CITEseq, Chem-Seq, DNAase-Seq, Hi-C, DAM-ID, TIRF microscopy (https://doi.org/10.1038/s4158), Split-seq. Preferably, specific epigenetic modifications, such as histone modification can be identified by single-cell epigenetic profiling using a microfluidic system as described in the examples of the present application.

In some embodiments, said at least one histone modification is present in at least 0.01%, preferably 0.05, 0.1, 0.2, 0.3, 0.4, 0.5, 1%, more preferably between 0.5% to 20% of tumor cells of the untreated sensitive tumor from which said drug-resistant tumor is derived.

In another embodiment, the method for determining the resistance to treatment of a cancer type with a cancer drug to a patient comprises determining in said patient tumor sample the expression level of at least one gene encoding by a biomarker as described above comprising one or more genomic sequence(s) selected from the list of Table 1 and 4.

Typically, the expression level of gene encoded by said biomarker in a patient sample is deemed to be higher or lower than the predetermined value obtained from the general population or from healthy subjects if the ratio of the expression level of said gene encoded by said biomarker in said patient to that of said predetermined value is higher or lower than 1.2, preferably 1.5, even more preferably 2, even more preferably 5, 10 or 20.

As used herein, the term “predetermined value of a biomarker” refers to the amount of the biomarker in biological samples obtained from the general population or from a selected population of subjects. For example, the general population may comprise apparently healthy subjects, such as individuals who have not previously had any sign or symptoms indicating the presence of cancer. The term “healthy subjects” as used herein refers to a population of subjects who do not suffer from any known condition, and in particular, who are not affected with any cancer. In another example, the predetermined value may be of the amount of biomarker obtained from selected population of subjects having an established cancer but who shows a clinically significant relief in a cancer type when treated with a cancer drug as described above. The predetermined value can be a threshold value, or a range. The predetermined value can be established based upon comparative measurements between apparently healthy subjects and subjects with established cancer.

The expression level of said gene encoded by a biomarker may be determined by any suitable methods known by skilled persons. Usually, these methods comprise measuring the quantity of mRNA or protein. Methods for determining the quantity of mRNA are well known in the art. For example the mRNA contained in the sample is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. In a preferred embodiment, the mRNA expression level is measured by RNA seq method, more preferably by single-cell RNA-seq. RNA seq can be used to analyse the cellular transcriptome. RNAseq, preferably single cell RNA seq can be performed for example in plate, micro or nano-wells, droplet-based microfluidics, microfluidics, tubes. The approach aims at deciphering tissue, sample, cell heterogeneity by interrogating genetic expression of whole transcriptome, or subset of gene, and classifying cells based on similar/closest transcriptomic expression pattern. The approach can also include as well interrogation of protein expression using nucleic acid tagged antibody against cell surface protein. One example of single cell RNA-seq method is illustrated in the FIGS. 1 and 2.

The level of the protein may be determined by any suitable methods known by skilled persons. Usually, these methods comprise contacting a cell sample, preferably a cell lysate, with a binding partner capable of selectively interacting with the protein present in the sample. The binding partner is generally a polyclonal or monoclonal antibodies, preferably monoclonal. The quantity of the protein may be measured, for example, by semi-quantitative Western blots, enzyme-labelled and mediated immunoassays, such as ELISAs, biotin/avidin type assays, radioimmunoassay, immunoelectrophoresis or immunoprecipitation or by protein or antibody arrays. The reactions generally include revealing labels such as fluorescent, chemiluminescent, radioactive, enzymatic labels or dye molecules, or other methods for detecting the formation of a complex between the antigen and the antibody or antibodies reacted therewith.

The term “patient sample” means any biological sample derived from a patient. Examples of such samples include fluids, tissues, cell samples, organs, biopsies, etc. Preferred biological samples are tumor sample.

The tumor sample may be from a patient tumor biopsy or a patient-derived xenograft (PDX) model of the cancer as disclosed in the examples of the present application. Drug-resistant tumor cells may be isolated directly from sample of patient drug-resistant tumor or generated from sample of patient untreated sensitive tumor or patient-derived xenograft by several rounds of drug treatment as disclosed in the examples of the present application. Various PDX models of cancers are available in the art. PDX models useful to perform the method of the present invention include with no limitations: luminal ER+ breast cancer (HBCx-22; Cottu et al., Breast Cancer Res. Treat., 2012, 133, 595-606) and derived Tamoxifen resistant cells (HBCx-22-TamR; Cottu et al., Clin. Cancer Res., 2014, 20, 4314-4325); Triple-negative breast cancer (HBCx-95; Cottu et al., Clin. Cancer Res., 2014, 20, 4314-4325; Marangoni et al., Clin. Cancer Res., 2018, 24, 2605-2615).

In some embodiments, said patient-derived tumor cells are from breast cancer, ovarian cancer, lung carcinoma, colorectal cancer, prostate cancer, pancreatic cancer and melanoma. Breast cancer include estrogen receptor positive (ER+), progesterone positive (PR+), HER2 positive (HER2+) and triple-negative (ER−, PR−, HER2−) breast cancer. In some preferred embodiments, said patient-derived tumor cells are from breast cancer, preferably triple-negative breast cancer.

“Triple-negative breast cancer” refers to any breast cancer that does not overexpress the genes for estrogen receptor (ER), progesterone receptor (PR) and HER2/Neu. This subtype of breast cancer is clinically characterized as more aggressive and less responsive to standard treatment and associated with poorer overall patient survival.

The presence of the biomarker(s) in the patient sample indicates that the patient is likely to be resistant to said cancer treatment, whereas the absence of the biomarker(s) indicates that the patient is likely to be responsive to said cancer treatment.

In some advantageous embodiments, the method comprises detecting at least one histone modification in at least one genomic sequence selected from the list of Table 1 to 6, and determining therefrom whether or not said patient is likely to be resistant to said cancer-drug treatment.

In another aspect, the present invention relates to a method for predicting the resistance to treatment of a cancer type with a cancer drug prior administration of said treatment to a patient, comprising:
i) detecting a histone modification of at least one biomarker according to the invention in a tumor sample that has been collected from the patient before beginning of treatment; and
ii) determining from the presence or absence of histone modification of said biomarker, whether the patient is likely to be resistant or sensitive to said treatment.

In some advantageous embodiments, the method further comprises a step of classification of the patients into resistant and sensitive group based on the presence or absence of the epigenetic biomarker(s) according to the invention.

In some advantageous embodiments, when the patient is found likely to be resistant to said cancer-drug treatment, the method further includes administering a therapeutically effective amount of a compound that modulates the epigenetic status of the genomic region of interest comprising the epigenetic modification, to reduce the development of resistance to cancer treatment in said patient.

Therefore, the use of the prediction method of the invention increases the efficiency of cancer treatment by reducing the development of resistance to cancer treatment.

Combined Therapy to Reduce the Development of Drug-Resistance

In connection with the above method of prediction of resistance to cancer treatment, the present invention relates to a combined preparation comprising a cancer drug and a compound that modulates the epigenetic status of the biomarker as described above for use in cancer treatment to reduce the development of resistance to said cancer treatment. In some preferred embodiments, said combined preparation is used in a cancer patient previously classified as resistant to treatment with said cancer drug using the method for determining resistance to cancer treatment according to the invention.

The present invention relates also to a method of treating a cancer patient comprising administering to said patient a therapeutically effective amount of a cancer drug and a therapeutically effective amount of a compound that modulates the epigenetic status of the biomarker. In some preferred embodiments, said combined preparation is administered to a cancer patient previously classified as resistant to treatment with said cancer drug using the method for determining resistance to cancer treatment according to the invention.

As used herein, a “therapeutically effective amount” or an “effective amount” means the amount of a composition that, when administered to a subject for treating a state, disorder or condition is sufficient to effect a treatment. The therapeutically effective amount will vary depending on the compound, formulation or composition, the disease and its severity and the age, weight, physical condition and responsiveness of the subject to be treated.

Such compounds that modulate the epigenetic status of a genomic region of interest include histone deacetylase (HDAC) inhibitor, DNA methyltransferase (DNMT) inhibitors, and Histone Methyl Transferase (HMT) inhibitors. In a particular embodiment, said compound is a DNA demethylase inhibitor.

As used herein, a “demethylase inhibitor” is any agent capable of partially or fully inhibiting one or more of the biological activities of a histone demethylase protein including, without limitation, a polypeptide, a polynucleotide, or a small molecule. Histone demethylases are a family of enzymes that catalyze the removal of methyl groups from lysine and arginine residues on histone tails. A demethylase inhibitor, can be KDM1 inhibitors or JmjC KDM inhibitors.

In a more particular embodiment, Histone Lysine Demethylase (KDM) inhibitor which can be used includes but is not limited to tranylcypromine ((trans-2-phenylcyclopropyl-1-amine, trans-2-P CPA)) and analogs thereof, e.g., with substitutions at the benzene ring, e.g., tranylcypromine 7, trans-2-PCPA analogue 28 (trans-2-pentafluorophenylcyclopropylamine, 2-PFPA), and trans-2-PCPA analogues carrying 4-bromo, 4-methoxy, and 4-trifluoromethoxy substitutions at the benzene ring (see, e.g., Gooden et al, Bioorg Med Chem Lett. 2008; 18(10):3047-51; Binda et al, J Am Chem Soc. 2010; 132(19):6827-33; Mimasu et al, Biochemistry. 2010; 49(30):6494-503; Benelkebir et al, Bioorg Med Chem. 2011; 19(12):3709-16; and Mimasu et al. Biochem Biophys Res Commun. 2008; 366(1): 15-22) or other inhibitors, e.g., 2,4-pyridinedicarboxylic acid (2,4-PDCA) (see, e.g., Kristensen et al, FEBS J. 2012 June; 279(1 1): 1905-14), and inhibitors of jumonji C (jmjC)-containing KDMs, e.g., 5-Carboxy-8-hydroxyquinoline (IOX1) and n-octyl ester thereof, as described in Schiller et al, Chem Med Chem. 2014 March; 9(3):566-71, or other inhibitors, as described in Spannhoff et al, Chem Med Chem. 2009; 4(10):1568-82; Varier and Timmers, Biochim Biophys Acta. 2011; 1815(1):75-89; Luo et al, J Am Chem Soc. Jun. 22, 2011; 133(24): 9451-9456; and Rotili et al, J Med Chem. 2014 Jan. 9; 57(1):42-55.

A number of HDAC inhibitors are known in the art, including but not limited to: Sodium Butyrate, Trichostatin A, hydroxamic acids, cyclic tetrapeptides, trapoxin B, depsipeptides, benzamides, electrophilic ketones, aliphatic acid compounds, pyroxamide, valproic acid, phenylbutyrate, valproic acid, hydroxamic acids, romidepsin; CI-994 (N-acetyldinaline, also tacedinaline); vorinostat (SAHA), belinostat (PXD101), LAQ824, panobinostat (LBH589), Entinostat (SNDX-275; formerly MS-275), EVP-0334, SRT501, CUDC-101, JNJ-26481585, PC124781, Givinostat (ITF2357), and mocetinostat (MGCD0103).

A number of DNMT inhibitors are known in the art, including but not limited to azacytidine, decitabine, Zebularine (1-(β-D-ribofuranosyl)-1,2-dihydropyrimidin-2-one), procainamide, procaine, (−)-epigallocatechin-3-gallate, MG98, hydralazine, RG108, and Chlorogenic acid. See also Gros et al, Biochimie. 2012 November; 94(11):2280-96.

A number of EZH2/HMT inhibitors are known in the art, including but not limited to: EPZ005687; E7438; Ell (Qi et al, 2012, supra); EPZ-6438; GSK343; BLX-01294, U C0638, BRD4770, EPZ004777, AZ505 and PDB 4e47, and those described in Garapaty-Rao et al, Chem. Biol. 20(11): 1329-1339 (2013); Ceccaldi et al, ACS Chem Biol. 2013 Mar. 15; 8(3):543-8; US 20130303555; and WO2012/005805; see, e.g., Wagner and Jung, Nature Biotechnology 30:622-623(2012), and Yao et al., J Am Chem Soc. 2011 Oct. 26; 133(42): 16746-9. In some embodiments, inhibitors that act on the G9A H3K9 methyltransferase, are used, e.g., BIX-01294 or BRD4770.

The cancer drug is any drug for chemotherapy or targeted therapy as disclosed above. In some preferred embodiments, said cancer drug is Capecitabine, Tamoxifen, or others.

The cancer is any cancer type as disclosed above. In some embodiments said cancer is selected from the group consisting of: breast cancer, ovarian cancer, lung carcinoma, colorectal cancer, prostate cancer, pancreatic cancer and melanoma. In some preferred embodiments, said cancer is breast cancer, preferably triple-negative breast cancer.

The compound that modulates the epigenetic status of the genomic region of interest and the cancer drug may be used simultaneously, separately or sequentially. The compound that modulates the epigenetic status of the genomic region of interest may be administered before, after, or concurrently with the therapeutic drug. Preferably, to reduce the resistance to cancer treatment, the compound that modulates the epigenetic status of the genomic region of interest may be administered prior to the cancer drug by at least 6 hours, 12 hours, 1 days, 2 days, 3 days, 5 days, 1 week.

The compounds or cancer drugs described herein may be administered by any means known to those skilled in the art, including, without limitation, intravenously, orally, intra-tumoral, intra-lesional, intradermal, topical, intraperitoneal, intramuscular, parenteral, subcutaneous and topical administration. Thus the compositions may be formulated as an injectable, topical, ingestible, or suppository formulation. Administration of the compounds or therapeutic agents to a subject in accordance with the present invention may exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage of compounds or cancer drugs administered in any given case will be adjusted in accordance with the composition or compositions being administered, the volume of the composition that can be effectively delivered to the site of administration, the disease to be treated or inhibited, the condition of the subject, and other relevant medical factors that may modify the activity of the compositions or the response of the subject, as is well known by those skilled in the art.

For example, the specific dose of compounds or cancer drugs for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol. The compositions can be given in a single dose schedule, or in a multiple dose schedule.

Suitable dosage ranges for a compound that modulates the epigenetic status and/or cancer drug may be of the order of several hundred micrograms of the agent with a range from about 0.001 to 10 mg/kg/day, preferably in the range from about 0.01 to 1 mg/kg/day.

The invention will now be exemplified with the following examples, which are not limitative, with reference to the attached drawings in which:

### FIGURE LEGENDS

FIG. 1: High throughput droplet-based microfluidics for single-cell RNA seq. Cells are diluted at optimal concentration to be encapsulated and to minimize cells being encapsulated with a second/third cell. The lysis reagents and reverse transcription (RT) reagents, possibly including other reagent for performing RACE (Rapid Amplification of cDNA Ends) amplification, are merged at a microfluidics junction and are co-encapsulated in sub or nanoliter volume droplet together with solid material, often in the form of hydrogel beads. These beads are used as solid support for single cell barcode (indexing) to be transferred to single cell DNA. The loading of >90% of droplet with beads allow recovery of most cells information.

FIG. 2: The encapsulation of hydrogel bead. The beads, because of their physical and chemical properties, are closely packed into a microfluidic inlet (left panel), are loaded 1:1 into droplet (middle and right panels), thus ‘beating’ Poisson statistics law.

FIG. 3: Barcoded bead production and quality control. (a) Beads were produced in a microfluidic device with 2-inlets by dispersing a mixture comprising PolyEthylene Glycol Di-Acrydrite (PEG-DA), Streptavidin Acrylamide and the photo-initiator. Flow rates were adjusted to produce 9 pl droplets, and immediately exposed to UV light for polymerization of the hydrogel network. Scale bar corresponds to 25 μm. (b) Split-and-pool synthesis principle for the addition of successive indexes. (c) Barcodes were synthesized by successive ligation of double-stranded indexes containing 5′ overhang of 4 base pairs by three rounds of split-and-pool synthesis using 96 Index 1, 96 Index 2 and 96 Index 3. Barcodes were flanked at one end by common sequences comprising a ½ Pac restriction site, a T7 promoter and the Illumina Read #2 sequencing primer, which were bound to the beads via a photocleavable linker (PC-linker). A 3′ C3-spacer was added to the 3′end of the photocleaved site for directed ligation to the other end of the barcode comprising a second common sequence with the ½ Pac restriction site ligated to the index 3. (d) Barcodes that failed in one of the three split-pool rounds were completed with a “block” oligonucleotide comprising a 5′ C3-spacer and a 3′ Inverted ddT to prevent ligation.

FIG. 4: Sequencing library preparation. (a) Enriched barcoded nucleosomes were linearly amplified by in vitro transcription. The amplified RNAs were reverse transcribed into cDNA by random priming, appending a reverse complement of Illumina Read #1 sequencing primer. The cDNAs were amplified by PCR, appending an Illumina P7 and P5 sequences. (b) Schematic of the final sequencing product with size in bp of each element constituting the sequence. (c) Electropherogram showing the size distribution of the final sequencing library post agarose gel purification. The smear ranges from 300 bp to 700 bp and corresponds to barcoded nucleosomes (profile obtained by Tapestation). (d) Single-cell ChIP-seq libraries were sequenced as follows: 50 bp were assigned to read the nucleosomal sequence and 100 bp were assigned to read the barcode.

FIG. 5: Sensitive and drug-resistant specific H3K27me3 chromatin landscapes in PDX model of triple negative breast cancer treated with Capecitabine. (a) Hierarchical clustering and corresponding heatmap of cell-to-cell Pearson correlation scores for scChIP-seq datasets. Sample color code is dark grey for HBCx-95 and light grey for HBCx-95-CapaR and the unique read count is indicated above heatmap. (b-c) t-SNE representation of scChIP-seq datasets, cells are colored according to the sample of origin (b) and consensus clustering segmentation (c). (d) Item consensus score in respect to Chrom_c2, a score of 1 corresponds to a cell as highly representative of Chrom_c2 cluster. Dotted lines represent item consensus score of 0.9 relative to Chrom_c2 (left line) or Chrom_c1 (right line). Dark grey cells originate from HBCx-95, light grey cells from HBCx-95-CapaR. Triangles highlight cells with a consensus score over 0.9 and in opposition to their sample of origin. (e) Volcano plot representing adjusted p-values (Wilcoxon rank test) versus log 2 fold-changes for differential analysis comparing chromatin enrichment between Chrom_c2 and c1 (thresholds of 0.01 for q-value and 1 for log 2FCJ). (f) Left panel: Pie chart representing the number of differentially enriched windows overlapping a TSS and with detectable transcription. Right panel: Log 2 expression fold-change between cells from HBCx-95-CapaR and HBCx-95 for all detected genes (n=37) within differentially enriched loci. Barplot is colored according to log 2FC and associated q-value (black for q>0.01, dark grey for significantly under-expressed and light grey for significantly over-expressed). (g-h) Left panels: aggregated H3K27me3 chromatin profiles for each cluster are shown for IGF2BP3 and COL4A2. The number and the percentage of cells with H3K27me3 enrichment within each cluster are indicated above tracks. Right panels: t-SNE plots representing scRNA-seq datasets, points are colored according to cell expression signal for IGF2BP3 or COL4A2. (i) Aggregated H3K27me3 chromatin profiles for HOXD locus depleted in H3K27me3 in Chrom_c2, but with no detectable transcription with scRNA-seq.

FIG. 6: Clustering of single-cell ChIP-seq H3K27me3 profiles and scRNA-seq profiles of human tumor cells from the HBCx-95 model. (a) Top panel: Plot of copy number in 0.5 Mb non-overlapping regions in Capecitabine-resistant PDX (HBCx-95-CapaR) versus untreated PDX (HBCx-95), obtained from the input of the bulk ChIP-seq experiments. Bottom panel: snapshots of loci affected by copy number variation for bulk DNA profiles of Capecitabine-resistance PDX and untreated PDX indicated in gray. (b) Left panel: hierarchical clustering and corresponding heatmap of cell-to-cell Pearson's correlation scores. Sample of origin (dark grey for HBCx-95 and light grey for HBCx-95-CapaR) and unique read count are indicated above the heatmap. Middle panel: t-SNE plots of scRNA-seq tumor cells, dots are colored according to the sample of origin and consensus clustering segmentation. Right panel: Consensus clustering scores for hierarchical clustering of scRNA-seq tumor cells. Consensus score ranges from 0 (white: never clustered together) to 1 (dark blue: always clustered together). Cluster membership is color coded beneath the dendrogram. (c) Consensus clustering analysis for scChIP-seq dataset. Left panel: mean of all pairwise correlation score between cluster's members is plotted for k clusters ranging from 2 to 10. At k=2 clusters, the intra-cluster correlation is maximized. Right panel: hierarchical clustering and corresponding heatmap of cell-to-cell consensus clustering scores for scChIP-seq on tumor cells (HBCx-95 and HBCx-95-CapaR PDXs). Consensus scores ranges from 0 (white: never clustered together) to 1 (black: always clustered together). Cluster membership is color coded above heatmap. (d) Barplot displaying the −log 10 of adjusted p-values from pathway analysis for regions with depletion of H3K27me3 in resistant cells. The gene sets are indicated on the barplot. (e) Left panels: Aggregated H3K27me3 chromatin profiles for Chrom_c1 and Chrom_c2 are shown for the loci identified in FIG. 5f, as significantly differentially enriched and expressed. For each window indicated in gray, the log 2 fold-change, the adjusted p-value (q-value), the number and the proportion of cells with H3K27me3 enrichment within each cluster are indicated. Right panels: t-SNE representation of scRNA-seq datasets. Dots are colored according to expression signal in each cell.

FIG. 7: A fraction of cells from sensitive tumor shares H3K27me3 chromatin features with resistant cells in a model of luminal ER+ PDX treated with Tamoxifen. (a) Hierarchical clustering and corresponding heatmap of cell-to-cell Pearson correlation scores for scChIP-seq datasets. Sample of origin is indicated in dark grey for HBCx-22 and light grey for HBCx-22-TamR, the unique read count is indicated above heatmap. (b) t-SNE representation of scChIP-seq datasets, cells colored according to sample of origin (left) and consensus clustering segmentation (right) (FIG. 8 c-d). (c) Item consensus score in respect to Chrom_c2. A score of 1 corresponds to a cell as highly representative of Chrom_c2 cluster. Dotted lines represent item consensus score of 0.9 relative to Chrom_c2 (upper line) or Chrom_c1 (lower line). (d) Pie chart representing the number of significantly differentially enriched (H3K27me3, q<0.01) windows overlapping a TSS and with detectable transcription. (e) Hierarchical clustering and corresponding heatmap of cell-to-cell Pearson correlation scores for scRNA-seq datasets. Sample of origin is indicated in dark grey for HBCx-22 and light grey for HBCx-22-TamR, the UMI count is indicated above heatmap. (f) t-SNE representation of scRNA-seq datasets, cells colored according to sample of origin (left) and consensus clustering segmentation (right) (FIG. 8f). (g) Hierarchical clustering of average expression scores per cell for each of the top 10 upregulated pathways (with lowest q-values) in HBCx-22-TamR versus HBCx-22. Sample of origin, RNA cluster and unique read count is indicated above heatmap. (h-i) Left panels: Snapshots for EGFR and IGFBP3 loci of aggregated H3K27me3 chromatin profiles for each cluster. For each window, log 2 fold-change and adjusted p-value are indicated. Middle panels: Barplots displaying the percentage of cells with H3K27me3 enrichment in each cluster. The corresponding number of cells is indicated above the barplots. For each cluster, the origin of cells (dark grey for HBCx-22 and light grey for HBCx-22-TamR) is indicated below. Right panels: Barplots displaying the average log 2 fold-change in EGFR and IGFBP3 expression level for cells in each cluster versus all remaining cells. The percentage of cells, within each cluster, with detectable EGFR or IGFBP3 expression is indicated above the barplot. For each cluster, origin of cells (dark grey for HBCx-22 and light grey for HBCx-22-TamR) is indicated below.

FIG. 8: Clustering of single-cell ChIP-seq profiles of human tumor cells from the HBCx-22 model. (a) Histograms of the distribution of scChIP-seq raw and unique sequencing reads per cell in untreated HBCx-22 and Tamoxifen-resistant HBCx-22-TamR PDX. (b) Copy number in 0.5 Mb non-overlapping regions plotted for bulk DNA profiles of Tamoxifen-resistant PDX (HBCx-22-TamR) versus untreated PDX (HBCx-22). No aberrant variation in copy number was identified in this xenograft model. (c) Consensus clustering analysis for scChIP-seq dataset. Left panel: mean of all pairwise correlation score between cluster's members is plotted for k clusters ranging from 2 to 10. At k=2 clusters, the intra-cluster correlation is maximized. Right panel: hierarchical clustering and corresponding heatmap of cell-to-cell consensus clustering scores for scChIP-seq on tumor cells (HBCx-22 and HBCx-22-TamR PDXs). Consensus scores ranges from 0 (white: never clustered together) to 1 (black: always clustered together). Cluster membership is color coded above heatmap. (d) Volcano plot representing adjusted p-values (Wilcoxon rank test) versus fold-changes for differential analysis comparing chromatin marks between Chrom_c2 and Chrom_c1 (q-value <0.01 and log 2FCJ >1). (e) Barplot displaying the −log 10 of adjusted p-values from pathway analysis for regions with depletion of H3K27me3 in cells from Chrom_c2. The gene sets are indicated on the barplot. (f) Hierarchical clustering and corresponding heatmap of cell-to-cell consensus clustering score for scRNA-seq tumor cells (HBCx-22 and HBCx-22-TamR PDXs). Consensus score ranges from 0 (white: never clustered together) to 1 (black: always clustered together). Cluster membership is color coded above the heatmap. (g) Left panel: aggregated H3K27me3 chromatin profiles for Chrom_c1 and Chrom_c2 are shown for the ALCAM locus. For each window indicated in gray, the log 2 fold-change and the adjusted p-value are indicated. Middle panel: barplot displaying the proportion of cells with H3K27me3 enrichment in each cluster. The corresponding number of cells is indicated above the barplot. For each cluster, the origin of cells (dark grey for HBCx-22 and light grey for HBCx-22-TamR) is indicated below. Right panel: barplot displaying the average log 2 fold-change for ALCAM expression level for cells in each cluster versus all remaining cells. The percentage of cells, within each cluster, with detectable ALCAM expression is indicated above the barplot. For each cluster, the origin of cells (dark grey for HBCx-22 and light grey for HBCx-22-TamR) is indicated below.

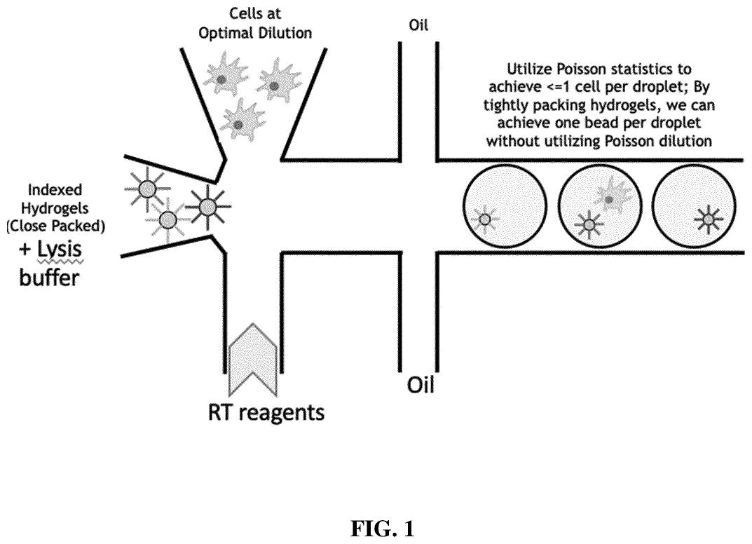

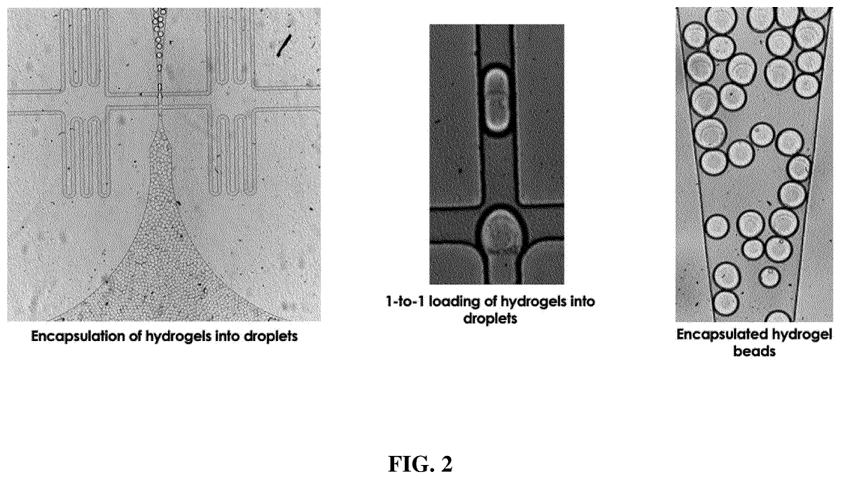

### EXAMPLES

Material and Methods

1. Cell Lines

Jurkat cells (ATCC, T18-125), an immortalized line of human T lymphocytes and Ramos cells (ATCC, CRL-1596), an immortalized line of human B lymphocytes, were grown in RPMI medium (ThermoFisher Scientific, #61870010) supplemented with 10% heat inactivated bovine serum (ThermoFisher Scientific, #16140071) and 1% Pen/Strep (ThermoFisher Scientific, #15140122). Mouse M300.19 cells (a gift from B. Moser), an immortalized line of mouse pre-B lymphocytes, were grown in RPMI 1640 medium (ThermoFisher Scientific, #61870010) supplemented with 10% fetal bovine serum (Fisher, #SH30070.03), 1% Pen/Strep (ThermoFisher Scientific, #15140122), 1% L-glutamine (ThermoFisher Scientific, #25030081) and 5×10⁻⁵ M P-mercapto-ethanol (ThermoFisher Scientific, #21985023).

2. Patient-Derived Xenografts (PDX)

Female Swiss nude mice were purchased from Charles River Laboratories and maintained under specific pathogen-free conditions. Their care and housing were in accordance with institutional guidelines and the rules of the French Ethics Committee (project authorization no. 02163.02). A PDX model of luminal breast cancer (HBCx-22) was previously established at Institut Curie from untreated early-stage luminal breast cancer with informed consent from the patient. Acquisition of a resistant phenotype for a derivative of HBCx-22, HBCx-22-TamR, was previously established and maintained. A PDX from a residual triple negative breast cancer post neo-adjuvant chemotherapy (HBCx-95) was previously established at Institut Curie with informed consent from the patient. HBCx-95 xenografts (n=6) were treated with Capecitabine (Xeloda, Roche Laboratories) orally at a dose of 540 mg/kg/day, 5 days a week for 6 weeks. Relative tumor volumes (RTV, mm3) were calculated. Mice with recurrent tumors were treated for a second round of Capecitabine when PDX reached a volume of over 200 mm³ (mice #35, #40 & #33). Mouse #40 did not respond to Capecitabine and PDX specimen was extracted at 1100 mm³ and tagged as HBCx-95-CapaR.

Prior to single-cell ChIP-seq, single-cell RNA-seq and bulk ChIP-seq, PDX were digested at 37° C. for 2 h with a cocktail of Collagenase I (Roche, #11088793001) and Hyaluronidase (Sigma, #H3506). Cells were further individualized at 37° C. using a cocktail of 0.25% trypsin/Versen (ThermoFisher Scientific, #15040-033), Dispase II (Sigma, #D4693) and Dnase I (Roche, #11284932001). Red Blood Cell lysis buffer (ThermoFisher Scientific, #00-4333-57) was then added to degrade red blood cells. To increase the viability of the cell suspension, dead cells were removed using the Dead Cell Removal kit (Miltenyi Biotec). Cells were re-suspended in PBS/0.04% BSA (ThermoFisher Scientific, #AM2616).

3. Single-Cell ChIP-Seq

3.1 Microfluidic Chips

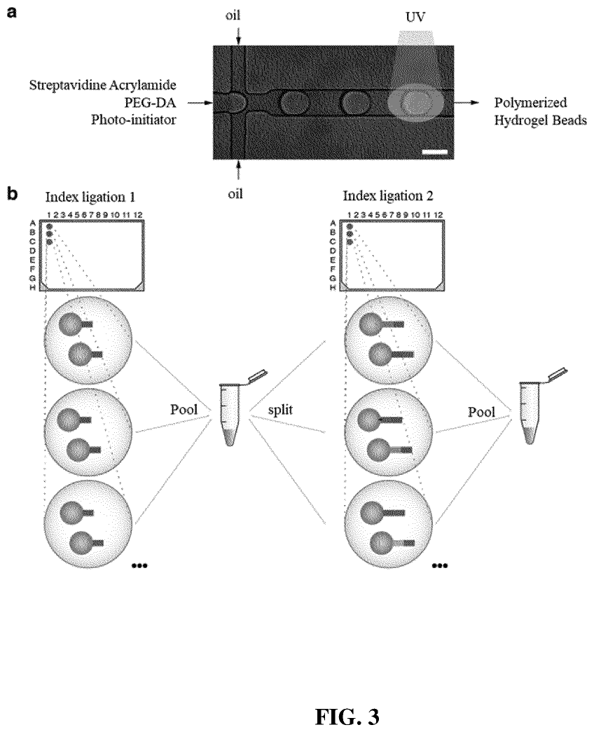

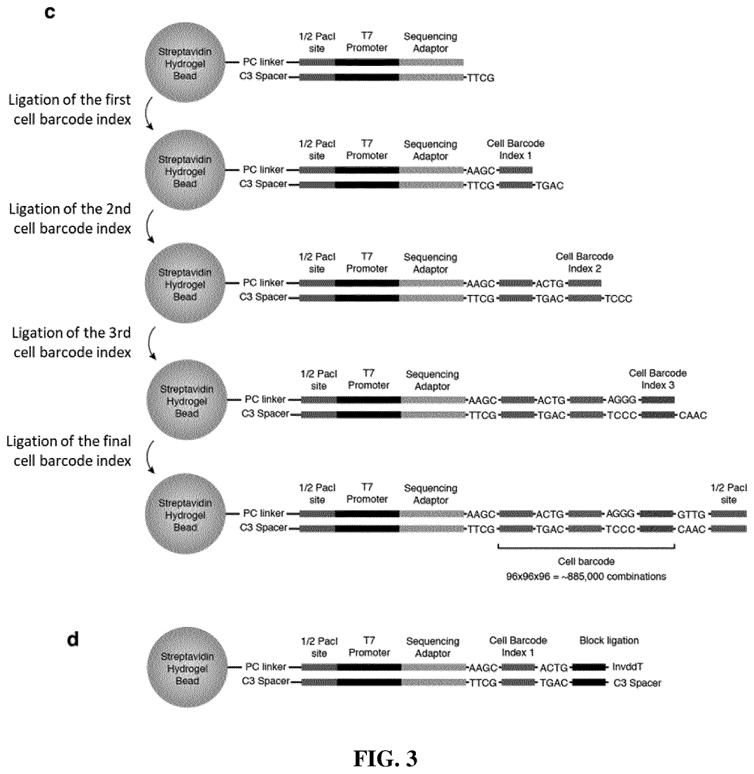

Four microfluidic chips were used: i) to compartmentalize single cells with lysis reagents and MNase in droplets; ii) to produce hydrogel beads; iii) to compartmentalize single hydrogel beads in droplets, and iv) for one-to-one fusion of droplets containing digested nucleosomes (from single lysed cells) with droplets containing single hydrogel beads (FIG. 3a). All chips were fabricated using soft-photolithography in poly-dimethylsiloxane (PDMS, Sylgard). Masters were made using one layer of SU-8 photoresist (MicroChem). The list depth of the photoresist layer for device I was 40.8±1 μm, for device ii was 30.0±1 μm and for device iii was 34.0±1 μm. For device iv, list depth was 45.0±1 μm and electrodes were prepared by melting a 51In 32.5Bi 16.5Sn alloy (Indium Corporation of America) into the electrode channels. Microfluidic devices were treated the day of the experiment with 1% v/v 1H,1H,2H,2H-perfluorodecyltrichlorosilane (ABCR, #AB111155) in Novec HFE7100 fluorinated oil (3M) to prevent droplets wetting the channel walls.

3.2 Microfluidic Operations

Droplet formation, fusion and fluorescence analysis was performed on a dedicated droplet microfluidic station, similar to Mazutis L. et al. 2013, Nat. Protoc. 8:873-891. The continuous oil phase for all droplet microfluidics experiments was Novec HFE7500 fluorinated oil (3M) containing 2% w/w 008-FluoroSurfactant (RAN Biotechnologies).

3.3 Cell Compartmentalization and Chromatin Digestion.

Cells were centrifuged (300 g, 5 min at 4° C.), labeled by 20 min incubation with 1 μM Calcein AM (ThermoFisher Scientific, #C3099). Then, cells are resuspended in cell suspension buffer, comprising DMEM/F12 (ThermoFisher Scientific) supplemented with 30% Percoll (Sigma, #P1644), 0.1% Pluronic F68 (ThermoFisher Scientific, #24040032), 25 mM Hepes pH 7.4 (ThermoFisher Scientific, #15630080) and 50 mM NaCl.

Cells were resuspended to give an average number of cells per droplets X of 0.1, resulting in 90.48% of empty droplets, 9.05% of droplets containing one cell and only 0.46% containing two or more cells due to Poisson distribution of the cells in droplets (Clausell-Tormos, J. et al. 2008. Chem Biol 15, 427-437). Overall, the inventors estimated that among non-empty droplets, 95.16% contained one cell and 4.84% contained two cells or more close to the expected values (94.92 and 5.08%, respectively).

The cells were co-flowed in a microfluidic chip (FIG. 3a) with digestion buffer containing lysis buffer (107.5 mM Tris-HCl pH 7.4, 322.5 mM NaCl, 2.15% Triton Tx-100, 0.215% DOC and 10.75 mM CaCl2)), 2 μM Sulforhodamine B (Sigma, #S1402-5G), 4 μM DY405 (Dyomics, #405-00), Protease Inhibitor cocktail and 0.2 U/μl Mnase enzyme (ThermoFisher Scientific, #EN0181). Droplets were produced by hydrodynamic flow-focusing (Anna, S. L. et al. 2003. Appl Phys Lett 82, 364-366) with a nozzle of 25 μm wide, 40 μm deep and 40 μm long. The flow rates (150 μl/hr for both aqueous phases, 850 μl/hr for the continuous oil phase) were calibrated to produce 45 μl droplets.

The droplets were collected in a collection tube (1.5 ml Eppendorf tube filled with HFE-7500 fluorinated oil) and then incubated at 37° C. for 20 min.

3.4 Production of Hydrogel Beads Carrying Barcoded DNA Adaptors

3.4.1 DNA Barcoding Strategy

Hydrogel beads carrying barcoded DNA adaptors were produced by split-and-mix synthesis using a method similar to that previously described in Zilionis, R. et al. 2017. Nat Protoc 12:44-73; Klein, A. M. et al. 2015. Cell; 161:1187-1201. Briefly, polyethylene diacrylate (PEG-DA) hydrogel beads containing streptavidin acrylamide were produced and barcoded primers were added to the beads by split-and-pool synthesis using ligation (FIG. 3b). PEG-DA hydrogel beads were produced using the microfluidic device indicated in FIG. 3a, essentially as Zilionis, R. et al. 2017. Nat Protoc 12:44-73.

The 9 μl droplets were produced at 4.5 kHz frequency and were exposed at 200 mW/cm2 with a 365 nm UV light source (OmniCure ac475-365) to trigger gel bead polymerization. Recovered gel beads were washed 10 times with washing buffer (100 mM Tris pH 7.4, 0.1% v/v Tween 20). Twelve million PEG-DA beads were incubated in 500 μl final volume for 1 h at room temperature with 50 μM of a photo-cleavable biotinylated dsDNA oligonucleotide (see SEQ ID NO: 1 and 2) and then distributed into a 96-well plate, each well containing 5 μl at 5 μM of a double-stranded DNA with a specific first index (index 1), for split-and-pool synthesis by ligation using T7 DNA ligase (NEB, #M0318) according to the manufacturer's instructions. At each round of split-and-pool, the hydrogel beads were pooled and washed (FIG. 3c). Repeating this splitting and pooling process 3 times in total (adding 3 indexes) results in 963 combinations, which generates ˜8.8×10⁵ different barcodes. After adding the last index, the beads were pooled, and a common double-stranded DNA oligo (SEQ ID NO: 3 and 4) was ligated to the beads (FIG. 3d). Each bead carries on average ˜5×10⁷ copies of a unique barcode.

3.4.2 Compartmentalization of Hydrogel Beads

The barcoded hydrogel beads were labeled by 30 min incubation with 10 μM Cy5-PEG3 biotin (Bioscience Interchim, #FP-1M1220) and washed with washing buffer (100 mM Tris pH 7.4, 0.1% v/v Tween 20), then suspended in bead mix (62.5 mM EGTA, 2 mM dNTPs, 1 mM ATP, 0.5 μM Sulforhodamine B). Barcoded hydrogel beads were co-flowed using the microfluidic device, with ligation mix (2× ligation buffer, 2 mM ATP, 1 μM Sulforhodamine B, 100 mM EGTA, 0.38 U/μl Fast-link DNA ligase [Lucigen, #LK0750H]) and EndRepair mix (4× ligation buffer, 4 mM dNTPs, 1 μM Sulforhodamine B, 0.08 U/μl Fast-link DNA ligase [Lucigen, #LK0750H], 0.15× ENDit repair mix [Lucigen, #ER0720]). The re-injection of close-packed barcoded hydrogel beads resulted in 65±5% of the droplets containing a single bead. The flow rates (150 μl/hr for the beads, 75 μl/hr for both ligation and EndRepair buffers, 150 μl/hr for the continuous oil phase) were calibrated to produce 100 pl droplets.

3.5 Fusion of Beads and Cell Droplets

Droplets containing fragmented chromatin and droplets containing barcoded hydrogel beads were re-injected into a microfluidic device with two aqueous inlets and one oil inlet for droplet fusion (FIG. 3a). The paired droplets were electro coalesced using an electric field generated by applying 100V ac (square wave) at 5 kHz across electrodes embedded in the microfluidic device. 75±5% of the droplets were correctly paired and fused.

3.6 Nucleosomes Barcoding in Droplets

Fused droplets were collected and exposed for 90 seconds at 200 mW/cm² with a 365 nm UV light source (OmniCure ac475-365). The ligation was performed at 16° C. overnight. The emulsion was then broken by addition of 1 volume of 80/20 v/v HFE-7500/1H,1H,2H,2H perfluoro-1-octanol (Sigma, #370533). The aqueous phase containing barcoded-nucleosomes was diluted by addition of 10 volumes of lysis dilution buffer (50 mM Tris-HCl pH 7.4, 1% Triton Tx-100, 0.1% DOC, 37.5 mM EDTA, 37.5 mM EGTA, 262.5 mM NaCl and 1.25 mM CaCl2)) and centrifuged 10 min at 10,000 g at 4° C. The soluble aqueous phase was used for the chromatin immunoprecipitation.

3.7 Immunoprecipitation of Barcoded-Nucleosomes

Protein-A magnetic particles (ThermoFisher Scientific, 10001D) were washed in blocking buffer comprising phosphate buffered saline (PBS) supplemented with 0.5% Tween 20, 0.5% BSA fraction V and incubated 4 hours at 4° C. in 1 ml blocking buffer with 2 μg of antibody (anti-H3K4me3 [Millipore, #07-473] and anti-H3K27me3 [Cell Signaling Technology, #9733]). After incubation, the particles were suspended with the barcoded-nucleosomes and incubated at 4° C. overnight. Magnetic particles were washed as described in Rotem, A. et al. 2015. Nat Biotechnol 33, 1165-1172 and immediately processed to prepare the sequencing library.

3.8 Sequencing Library Preparation & Sequencing

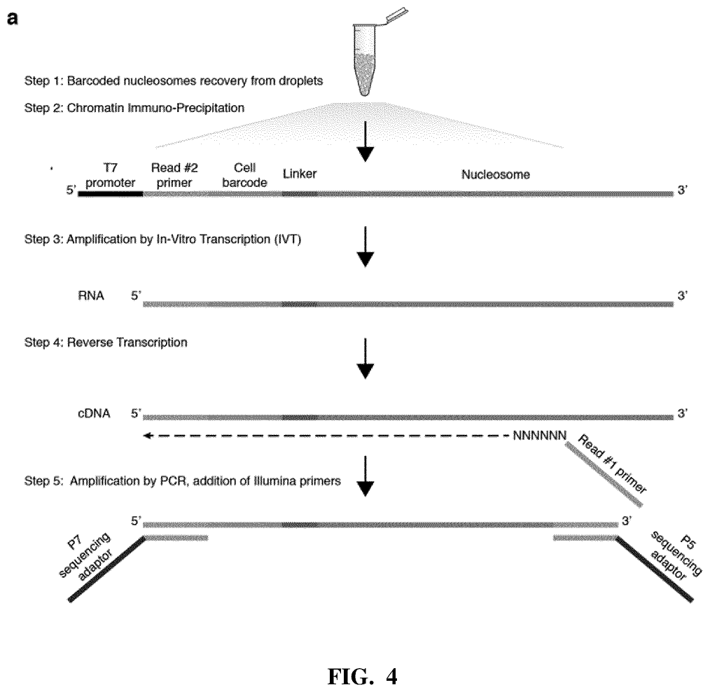

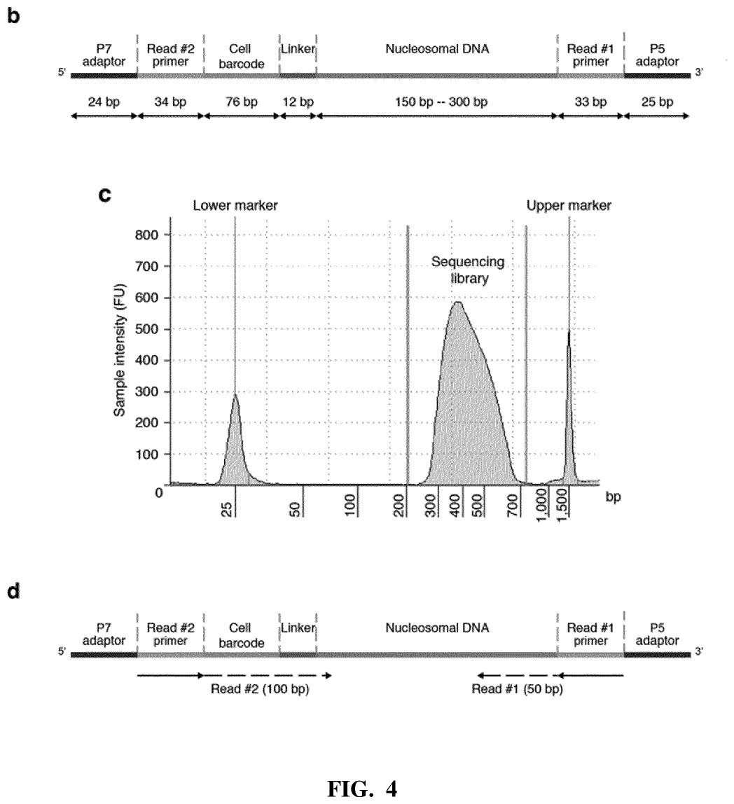

Concatemers of barcodes were digested by Pac restriction enzyme (NEB, #R0547), following the manufacturer's instructions. Immunoprecipitated chromatin was then treated with RNAse A (ThermoFisher Scientific, #EN0531) and with Proteinase K (ThermoFisher Scientific, #E00491). DNA was eluted from the magnetic particles with 1 volume of elution buffer (1% SDS, 10 mM Tris-HCl pH 8, 600 mM NaCl and 10 mM EDTA). Eluted DNA was purified with 1× AMPure XP beads (Beckman, #A63881) and eluted with RNAse/Dnase free water. Barcoded-nucleosomes were amplified by in vitro transcription using the T7 MegaScript kit (ThermoFisher Scientific, #AM1334). The resulting amplified RNA was purified using 1× RNAClean XP beads (Beckman, #A66514) and reverse transcribed using SEQ ID NO: 5 (FIG. 4a). After RNA digestion, DNA was amplified by PCR. The final product was size-selected by gel electrophoresis (FIG. 4c).

Single-cell ChIP-seq libraries were sequenced on an Illumina NextSeq 500 MidOutput 150 cycles. Cycles were distributed as follows: 50 bp (Read #1) were assigned for the genomic sequence and 100 bp (Read #2) were assigned to the barcode (FIG. 4d). The first 4 cycles of Read #2 were dark-cycles to prevent low complexity failure during clusters identification.

4. Single-Cell ChIP-Seq Data Analysis

Sequencing data were analyzed with Python (v2.7.12) and R (v3.3.3) using the reference genome hg38 (GenBank assembly accession: GCA_000001405.15 (Genome Reference Consortium Human Build 38 submitted in Dec. 17, 2013).

4.1 De-Multiplexing Cellular Barcodes

Barcodes were extracted from Reads #2 by first searching for the constant 4 bp linkers found between the 20-mer indices of the barcode allowing up to 1 mismatch in each linker (FIG. 4b). If the correct linkers were identified, the three interspersed 20-mer indices were extracted and concatenated together to form a 60 bp non-redundant barcode sequence. A library of all 884,736 combinations of the 3 sets of 96 indices (96³) was used to map barcode sequences using the sensitive read mapper Cushaw3 (Liu, Y. et al. PLoS One, 2014, 9: e86869). Each set of indices was error-correcting because it takes more than an edit-distance of 3 to convert one index into another. The inventors therefore set a total mismatch threshold of 3 across the entire barcode, with two or less per index to avoid mis-assigning sequences to the wrong barcode Id. In a second, slower step, sequences that could not be mapped to the Cushaw3 index-library were split into their individual indices and each index compared against the set of 96 possible indices, allowing up to 2 mismatches in each individual index. Any sequences not assigned to a barcode Id by these two steps were discarded.

4.2 Alignment, Filtering & Normalization

Reads #1 were aligned to mouse mm10 and human hg38 reference genomes using bowtie (v1.2.2) by keeping only reads having no more than one reportable alignments and 2 mismatches.

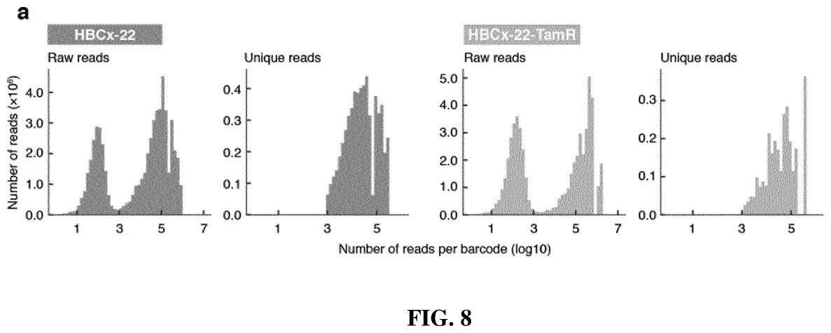

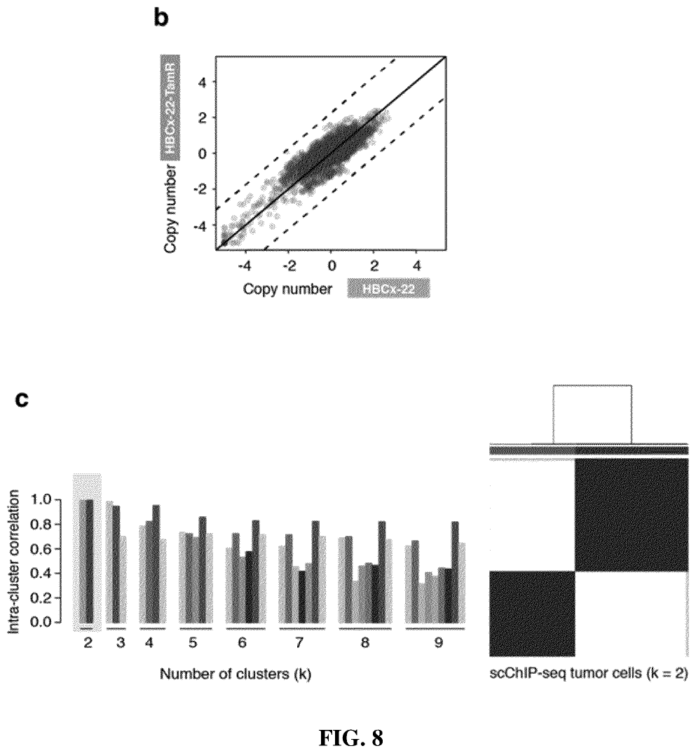

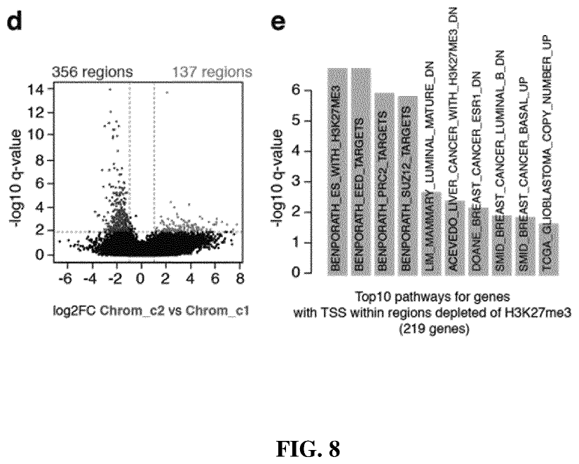

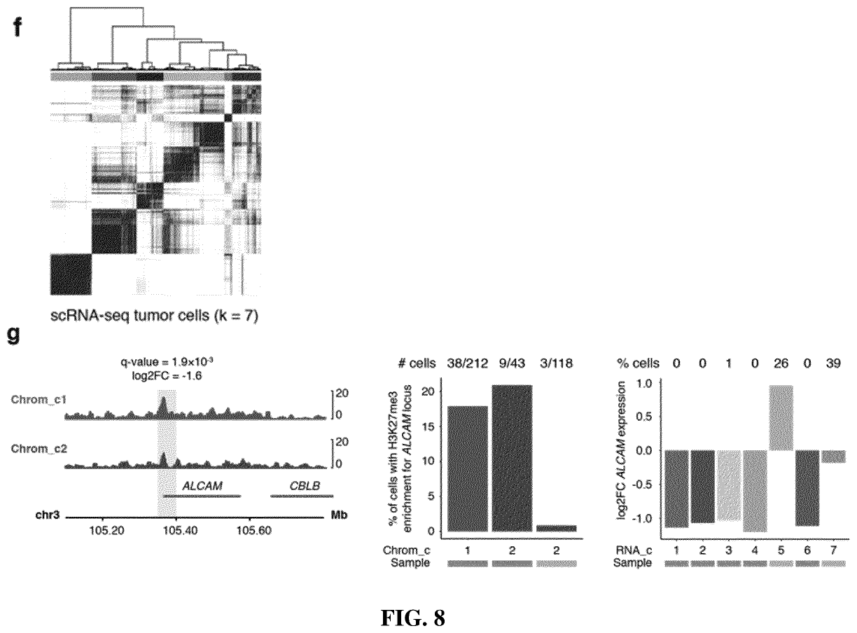

Raw reads are distributed according to a bimodal distribution, the lower peak most probably corresponding to droplets with barcoded beads but without cells (Rotem, A. et al. Nat Biotechnol; 2015, 33:1165-1172), and the right peak corresponding to droplets with cell with bead (FIG. 8a); thereby setting a read count cut-off to define barcodes associated to a cell. For subsequent analysis, the inventors kept barcodes with a unique (post PCR duplicate removal) read count above this cut-off. To remove PCR duplicates, for each barcode (i.e cell), all the reads falling in the same 150 bp window were stacked into one as reads possibly originating from PCR duplicates or from the same nucleosome. The inventors generated coverage matrix and metrics from these de-duplicated reads, referred to as ‘unique reads’ in the text.

For each cell, reads were binned in non-overlapping 50 kb for H3K27me3, known to accumulate over broad genomic regions, and 5 kb genomic bins for H3K4me3, known to accumulate in narrow peaks around transcription start sites, spanning the genome to generate a n×m coverage matrix with n barcodes and m genomic bins. The inventors combined coverage matrices for each of the four analyses from the following samples: (i) Ramos and Jurkat, (ii) mouse cells from HBCx95 and HBCx-95-CapaR, (iii) human cells from HBCx95 and HBCx95-CapaR, and (iv) human cells from HBCx-22 and HBCx-22-TamR.

The inventors first removed cells with a total number of uniquely mapped reads within the upper percentile, considered as outliers, and filtered out genomic regions not represented in at least 1% of all cells. By PCA analysis, the inventors could group cells independently of coverage only if cells had at least 1,600 unique reads per cell. For all subsequent analyses, the inventors excluded cells with lower coverage. Coverage matrices were then normalized by dividing counts by the total number of reads per cell and multiplying by the average number of reads across all cells.

4.3 Unsupervised Clustering of Single-Cell ChIP-Seq Profiles

Normalized matrices were reduced by principal component analysis (n=50 first components selected for further analysis). To improve the stability of the clustering approaches, the inventors further limited the analysis to cells displaying a Pearson's pairwise correlation score above a threshold t with at least 1% of cells. Threshold t was defined as the upper percentile of Pearson's pairwise correlation scores for a randomized dataset.

The inventors used consensus clustering, Bioconductor ConsensusClusterPlus package (Wilkerson, M. D. & Hayes, D. N. 2010. Bioinformatics 26, 1572-1573), to examine the stability of the clusters and compute item consensus score for each cell. The inventors established consensus partitions of the data set in k clusters (for k=2, 3, . . . ), on the basis of 1,000 resampling iterations (80% of cells) of hierarchical clustering, with Pearson's dissimilarity as the distance metric and Ward's method for linkage analysis. The optimal number of clusters (k) was chosen to maximize intra-cluster correlation score. Clustering results were visualized with t-SNE plots (Van der Maaten, L. & Hinton, G. 2008. J Mach Learn Res 9, 2579-2605). To visualize chromatin profiles of subpopulations, the inventors aggregated reads of single-cells within each cluster and created enrichment profiles using the R package Sushi (Phanstiel, D. H. et al. 2014. Bioinformatics. 30, 2808-2810).

4.4 Differential Analysis of Single-Cell ChIP-Seg Profiles

To identify differentially enriched regions across single-cells for a given cluster, the inventors performed a non-parametric Wilcoxon rank sum test comparing normalized counts from individual cells from one cluster versus all other cells. The inventors testes for the null hypothesis that the distribution of normalized counts from the two compared groups have the same median, with a confidence interval 0.95. The inventors limited analysis to the windows selected for unsupervised analysis above.

P-values were corrected for multiple testing using Benjamini-Hochberg procedure (Benjamini, Y. & Hochberg, Y. 1995. J R Stat Soc 57, 289-300). Genomic regions were considered as ‘enriched’ or ‘depleted’ for H3K27me3 or H3K4me3 if adjusted p-values, ‘q-values’, were lower than 0.01 and the absolute log 2 fold change greater than 1.

4.5 scRNA-Seq Comparison.

For H3K27me3 scChIP-seq analysis, the inventors used peak annotation from bulk ChIP-seq datasets to further annotate 50 kbp windows and corresponding genes: for each window, the inventors kept for subsequent analyses (gene annotation and scRNA-seq comparison), genes with a transcription start site (TSS) overlapped by a peak in any condition, using bedtools (v2.17)50 and the reference annotation of the human transcriptome Gencode_hg38_v26, limited to protein coding, antisense and lncRNA genes.

5. Bulk ChIP-seq

ChIP experiments were performed as described previously in Vallot, C. et al. 2015. Cell Stem Cell 16, 533-546, on 10⁶ cells from cell suspensions obtained above from HBCx-22, HBCx-22-TamR, HBCx-95 and HBCx-95-CapaR using anti-H3K27me3 antibody (Cell Signaling Technology, #9733). 2 ng of immune-precipitated and input DNA were used to prepare sequencing libraries using the Ovation Ultralow Library System V2 (Nugene) according to the manufacturer's instructions. Bulk ChIP-seq libraries were sequenced on an Illumina HiSeq 2500 in Rapid run mode SE50.

6. Bulk ChIP-Seq Data Analysis

Reads were aligned to mouse mm10 and human hg38 reference genomes using bowtie (v1.2.2) and the tool bamcmp was used to separate human from mouse sequences. Subsequent analysis was performed as explained previously. Only uniquely mapping reads were kept for the analysis, in addition PCR duplicates were removed using Picard Tools (https://broadinstitute.github.io/picard/). Data were binned in 5 kb (H3K4me3) or 50 kb (H3K27me3) consecutive genomic windows. For each window, log 2 RPM were computed, as the logged number of reads per millions of mapped reads.

7. Single-Cell RNA-Seq

Approximately 3,000 cells from each cell suspensions, HBCx-22, HBCx-22-TamR, HBCx-95 and HBCx-95-CapaR, were loaded on a Chromium Single Cell Controller Instrument (10× Genomics) according to the manufacturer's instructions. Samples and libraries were prepared according to the manufacterer's instructions. Libraries were sequenced on Illumina an HiSeq 2500 in Rapid run mode, using paired-end 26 bp-98 bp sequencing.

8. Single-Cell RNA-Seq Data Analysis

Single-cell sequencing files were processed using the Cell Ranger Single Cell Software Suite (v1.3.1) to perform quality control, sample de-multiplexing, barcode processing, and single-cell 3′ gene counting (http://software.10xgenomics.com/single-cell/overview/welcome) using the UCSC mouse (mm10) and human (hg19) transcriptome and genome with default parameters. 2,728 cells with an average coverage of 30,166 reads/cell (1,564 human and 1,191 mouse cells) for HBCx-22, 1,746 cells with an average coverage of 41,166 reads/cell (753 human and 1,013 mouse cells) for HBCx-22-TamR, 1,184 cells with an average coverage of 160,583 reads/cell (545 human and 647 mouse cells) for HBCx-95, 2,087 cells with an average coverage of 38,345 reads/cell (861 human and 1,242 mouse cells) for HBCx-95-CapaR were analyzed. Further analysis was performed in R (v3.3.3) using custom R scripts. Any cell with more than 10% of mitochondrial UMI counts was filtered out. The inventors only kept cells with a total count of UMI below 100,000 and of detected genes below 6,000 and over 1,000. The inventors then only kept genes with at least 1 transcript in at least 2 cells. Using the R package scater, scRNAseq count matrices were normalized for coverage and transformed by RLE, ‘Relative Log Expression’ method (McCarthy, D. J et al. Bioinformatics, 2017, 33:1179-1186). Using annotations from the R package ccRemover (Barron, M. et al. Sci Rep, 2016, 6:33892), the inventors removed genes related to cell cycle from subsequent clustering analyses to group cells according to cell identity and not cell-cycle related phenomena. Barnes-hut approximation to t-SNE was then performed on the n=50 first principal components (PCA) to visualize cells in a two-dimensional space. Clusters were identified using Consensus Clustering as for scChIP-seq analyses above. The inventors identified genes that were differentially expressed between clusters using edgeR GLM statistical models (Robinson, M. D. et al. Bioinformatics, 2010, 26:139-140). For stromal mouse cells, clusters were identified according to the differential expression of hallmark genes.

9. Copy Number Profiles of Bulk Tumor Cells

The R package HMMcopy was used to correct for copy number variation in non-treated versus resistant xenograft models. Reads from bulk input ChIP-seq samples were binned in 0.5 Mb non-overlapping regions spanning the genome. Regions with a deviation to the mean greater than n=2 standard deviations were removed for analysis.

Results

Characterization of Epigenetic and Transcriptional Makers

The inventors studied the heterogeneity of chromatin profiles among tumor cells from the same pair of triple-negative breast tumor samples (n=4,331 cells from HBCx_95 and HBCx_CapaR, with average coverage of 5,160 reads per cell).

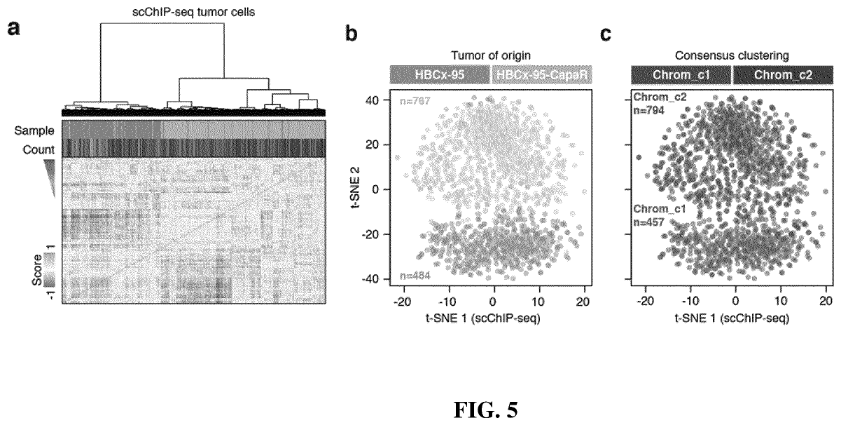

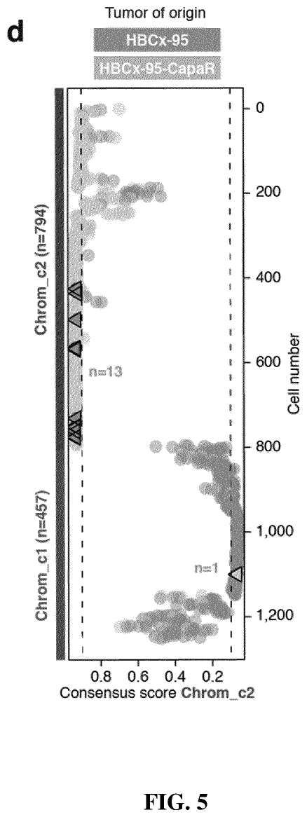

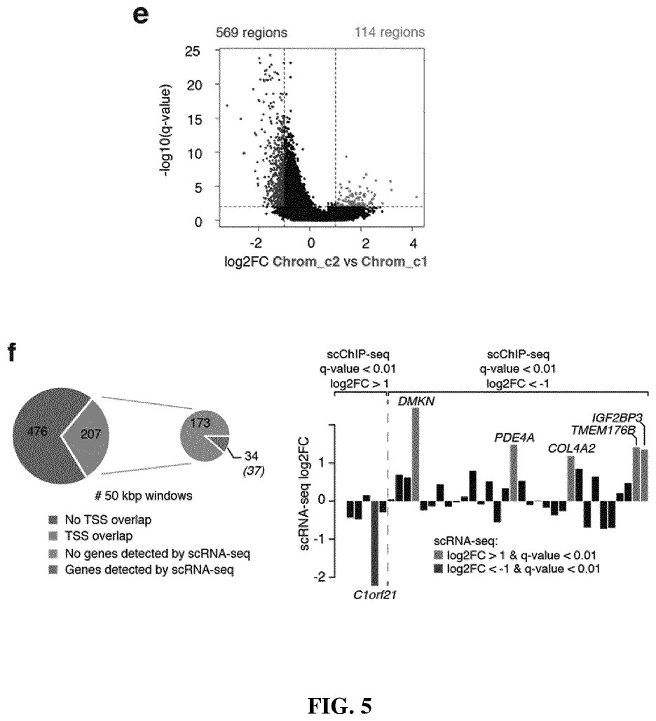

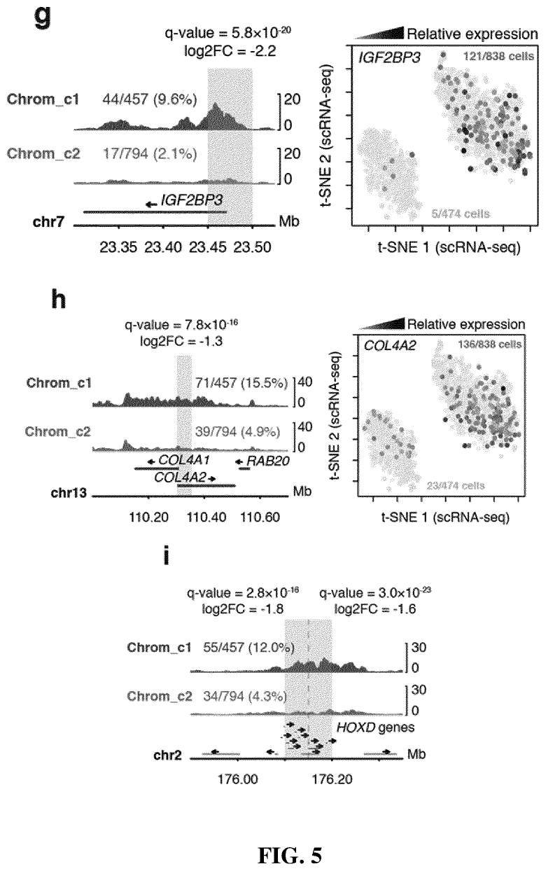

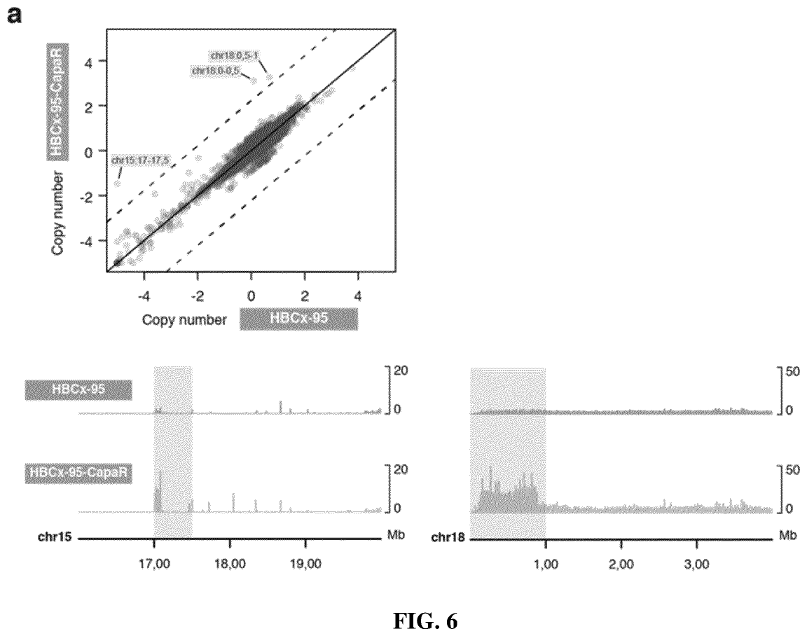

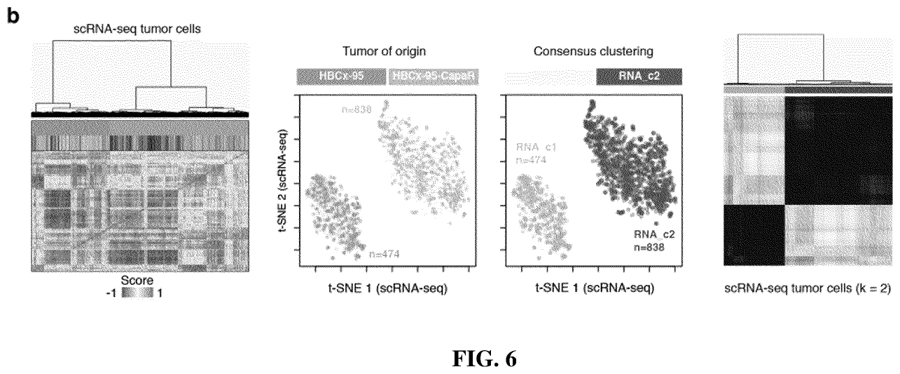

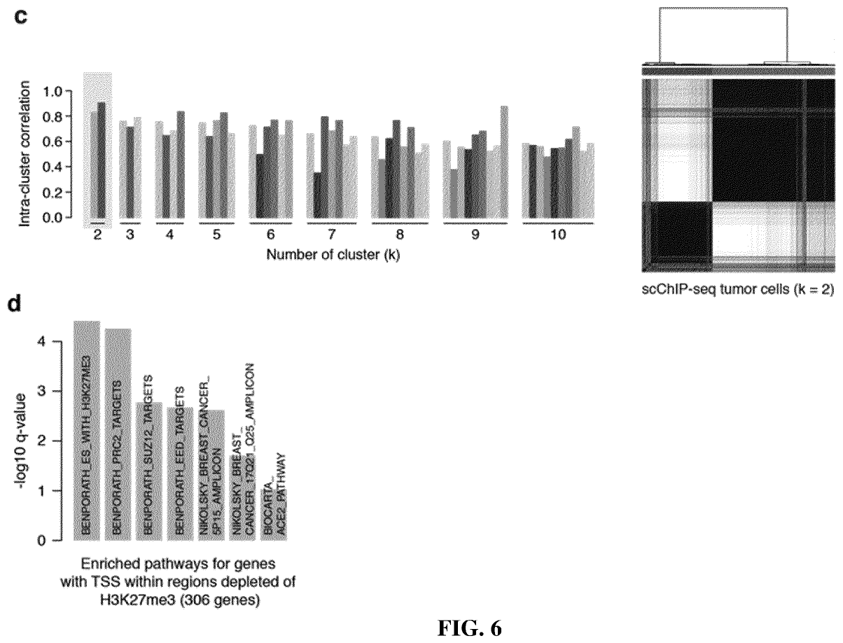

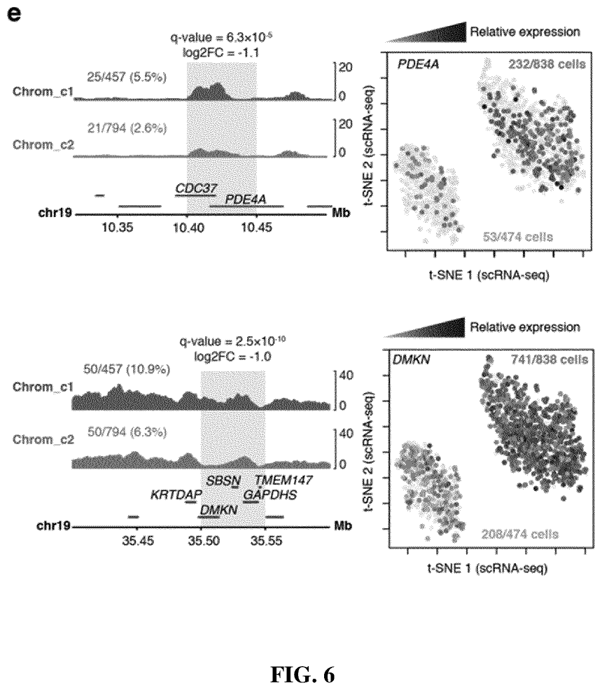

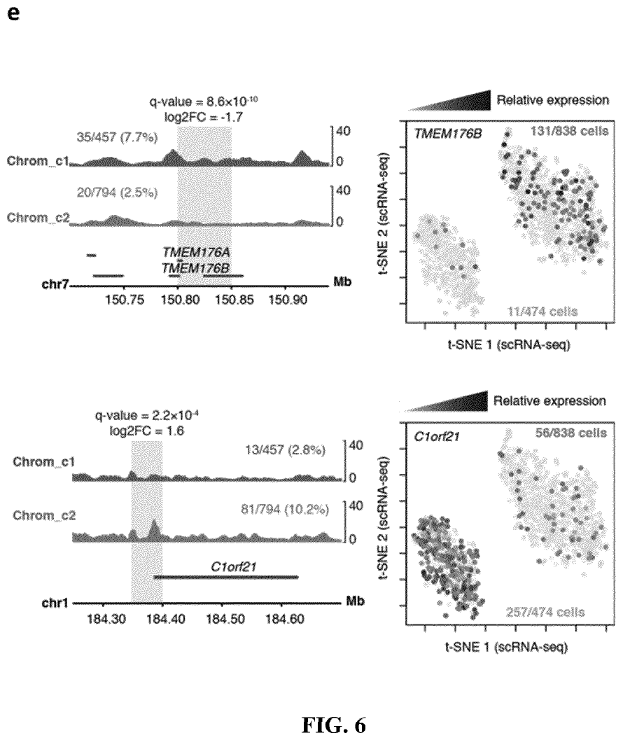

The inventors removed from the analysis loci affected by copy-number variations, as identified from bulk DNA profiles, to focus on chromatin alterations (FIG. 6a). Based on both chromatin and transcriptomic profiles, cells clustered primarily according to their sensitive or resistant tumor origin (FIG. 5a-c, FIG. 6b-c).

While the chromatin profiles of sensitive cells were largely homogeneous, distinct chromatin states within the resistant population were apparent (FIG. 5a), suggesting that heterogeneous populations of resistant cells, with distinct chromatin features, emerged.

However, consensus clustering also showed that 3% of cells of from the untreated tumor (n=13 out of 484) robustly classify with resistant cells (resistant-like cells) (FIG. 5d, Consensus Score over 0.9), suggesting that they share common chromatin features. Resistant and resistant-like cells, corresponding to Chrom_c2, displayed a high number of loci depleted in H3K27me3 enrichment compared to sensitive-like cells from Chrom_c1 (FIG. 5 e-f, n=569 loci with depleted versus 114 with enriched H3K27me3, q-value <0.01 and |log 2FC|>1, overlapping a transcription start site, see Table 1).

Loci specifically devoid of H3K27me3 in cells from Chrom_c2 were enriched in genes targets of the Polycomb complex (FIG. 6e), indicating that the inventors are observing a demethylation of expected EZH2 targets. The inventors could only detect transcription within 5% of these loci, either due to the absence of transcription or to insufficient sensitivity of the scRNA-seq procedure. Within these loci, six genes were significantly deregulated according to scRNA-seq, and all in accordance to their H3K27me3 chromatin states (FIG. 5f and FIG. 6e).

Interestingly, the inventors identified a genomic region including IGF2BP3, a gene known to promote resistance to chemotherapy ((FIG. 5g) (Lederer, M et al. Semin Cancer Biol, 2014; 29:3-12) and regions with genetic markers of epithelial-to-mesenchymal transition (COL4A1, HOXD cluster, FIG. 5h-i) (Zheng, X. et al. Nature, 2015; 527:525-530; Fischer, K. R. et al. Nature; 2015, 527:472-476), which induces resistance to chemotherapy.

In addition, the inventors profiled a pair of luminal ER+ breast PDXs: HBCx-22, responsive to Tamoxifen and HBCx-22-TamR, a tumor derivative with previously characterized acquired resistance to Tamoxifen. To obtain a high average coverage of 10,228 reads per cell, the inventors limited the number of encapsulated cells (n=822 tumor cells, FIG. 8a-b).

Tumor cells displayed two major chromatin profiles related to their tumor of origin.

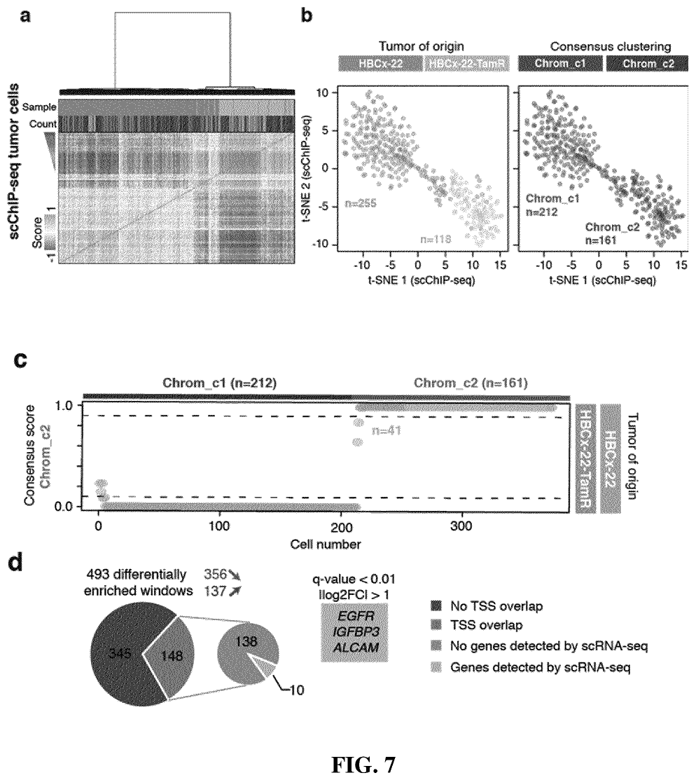

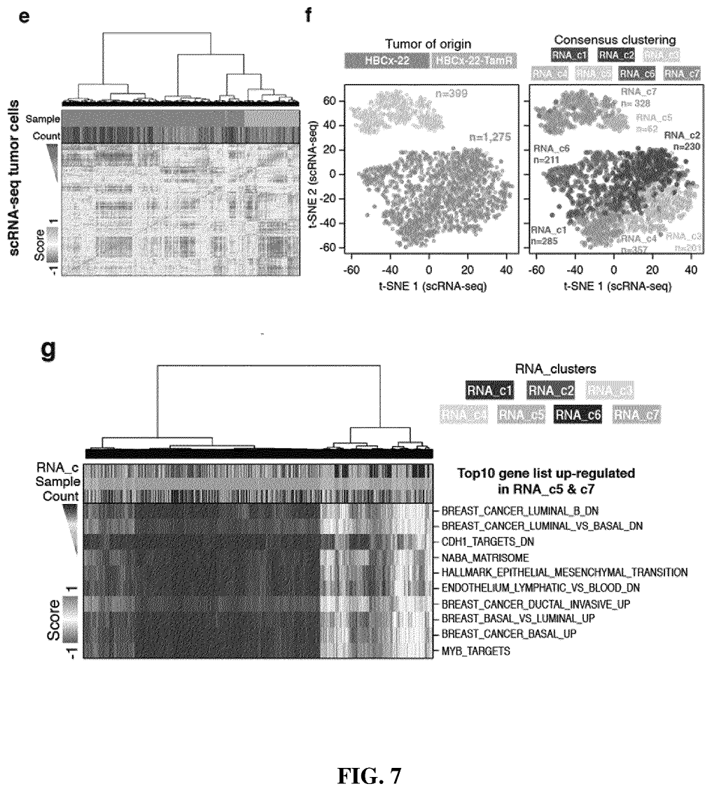

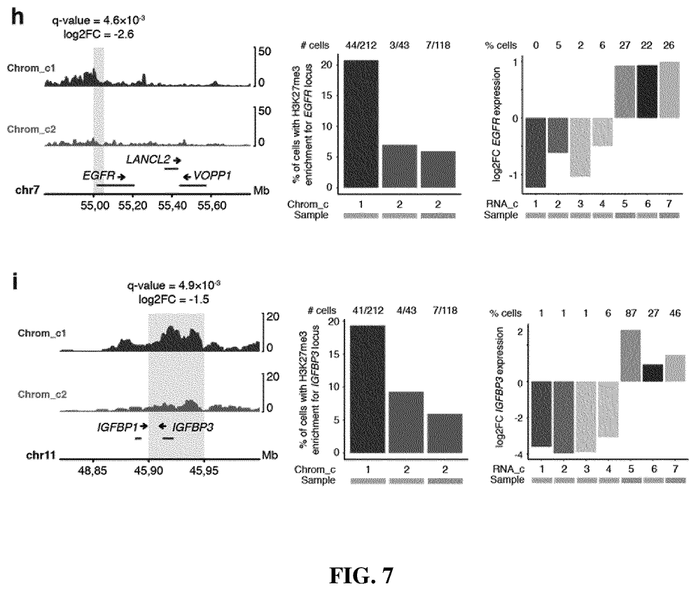

However, 16% (n=41 out of 255) of cells within the sensitive tumor shared chromatin features with all resistant cells (FIG. 7a-c and FIG. 8c). Chromatin features characteristic of resistant cells were, thus, already found in rare cells from the sensitive tumor (resistant-like cells), and could have been selected for by Tamoxifen treatment.

Differential analysis of chromatin features revealed that resistant-like cells have predominantly lost H3K27me3 marks compared to sensitive-like cells (FIG. 8d, n=356 loci with depleted versus 137 with enriched H3K27me3, see Table 4).

Loci specifically devoid of H3K27me3 in cells from Chrom_c2 were enriched in genes targets of the Polycomb complex, and characteristic of basal-like signatures of the mammary epithelium (FIG. 8e).

With scRNA-seq, the inventors could only detect transcription in 2% of differentially enriched windows, and significant differential expression for 3 genes, all showing transcription activation in a fraction of resistant cells in mirror to their loss of H3K27me3 enrichment: EGFR, a gene implicated in resistance to Tamoxifen (Massarweh, S. et al. Cancer Res; 2008, 68:826-833; Ciupek, A. et al. Breast Cancer Res Treat; 2015, 154:225-237), IGFBP3 and ALCAM (FIG. 7d, h-i and FIG. 8g).

Parallel scRNA-seq analysis of the same samples revealed several clusters of cells within the resistant and sensitive tumor (FIG. 7e-f, FIG. 8f). While no cells from the sensitive tumor clustered with the resistant cells, the inventors show that cells from the RNA_c6 cluster, originating from the sensitive tumor (corresponding to 17%, 211 out of 1,275 cells), display activation of the pathways characteristic of resistant tumor cells, among which basal-like gene signatures and signature of epithelial to mesenchymal transition (FIG. 7g). These observations independently confirm that non-genetic features common to resistant cells, either at the transcriptomic or chromatin-level, are already found in cells from the sensitive tumor.

Conclusions

Profiling histone modifications at the single-cell level with high coverage, up to 10,000 loci in average per cell, was instrumental to reveal the presence of relatively rare chromatin states within tumor samples. This study suggests that rare cells with chromatin features characteristic of resistant cancer cells exist before treatment and could be selected for by cancer therapy.

Spontaneous heterogeneity of chromatin states in untreated cells may be a key molecular component in the acquisition of drug-resistance, regardless of the mechanism of action of the cancer treatment: here Tamoxifen, targeting the estrogen receptor, and Capecitabine, a classical chemotherapy inhibiting the synthesis of thymidine monophosphate. This study permits to discover new biomarkers for patient stratification and opens up perspectives for novel therapeutic strategies both in luminal and triple-negative breast cancer to counteract resistance. For example, preventing loss of repressive chromatin marks such as H3K27me3, as observed in resistant cells here, by combining treatment with Capecitabine and Tamoxifen with drugs such as demethylase inhibitors could be a strategy to consider to minimize resistance.

TABLE 1
Genomic regions depicted in H3K27me3 enrichment in resistant and resistant-like cells compared to sensitive
cells for Capecitabine treatment overlapping a transcription start site, q-value < 0.01 (sixth column)
and |log2FC| > 1 (fifth column). The first column relates to the identification numbers of the
genomic sequences using the reference genome GRCh38 (GeneBank assembly accession GCA_000001405.15),
second column shows the chromosome number. The third and fourth columns relate to the start and end position
of genomic sequence respectively. Last column indicates genes related to transcription start site.

| | | | | log2FC. res. | adj. | Associated |
|---|---|---|---|---|---|---|
| ID | chr | start | end | vs. sens | pval | gene |
| chr19_35500000_35550000 | chr19 | 35500000 | 35550000 | −1.00452 | 2.46E−10 | DMKN; SBSN; GAPDHS |
| chr19_10400000_10450000 | chr19 | 10400000 | 10450000 | −1.10188 | 6.32E−05 | PDE4A; CDC37 |
| chr1_184350000_184400000 | chr1 | 1.84E+08 | 1.84E+08 | 1.582341 | 0.000221 | RP11-382D12.2; C1orf21 |
| chr7_150800000_150850000 | chr7 | 1.51E+08 | 1.51E+08 | −1.68136 | 8.56E−10 | TMEM176A; TMEM176B; AOC1 |
| chr17_81150000_81200000 | chr17 | 81150000 | 81200000 | −1.47673 | 3.79E−08 | AATK-AS1; AATK |
| chr9_133250000_133300000 | chr9 | 1.33E+08 | 1.33E+08 | −1.18304 | 3.19E−05 | ABO |
| chr19_53850000_53900000 | chr19 | 53850000 | 53900000 | −1.42565 | 2.11E−07 | AC008440.10; MYADM; PRKCG |
| chr2_176150000_176200000 | chr2 | 1.76E+08 | 1.76E+08 | −1.56875 | 2.95E−23 | AC009336.19; HOXD4; |
| | | | | | | RP11-387A1.5; HAGLROS; |
| | | | | | | HOXD1; HAGLR |
| chr2_16200000_16250000 | chr2 | 16200000 | 16250000 | −1.0729 | 1.8E−06 | AC010745.2; AC010745.3 |
| chr2_23350000_23400000 | chr2 | 23350000 | 23400000 | −1.13323 | 7.67E−14 | AC012506.3; AC012506.4; |
| | | | | | | KLHL29 |
| chr18_76650000_76700000 | chr18 | 76650000 | 76700000 | −1.08204 | 1.73E−06 | AC034110.1 |
| chr2_11100000_11150000 | chr2 | 11100000 | 11150000 | −1.07671 | 0.000243 | AC062028.1; C2orf50 |
| chr2_236150000_236200000 | chr2 | 2.36E+08 | 2.36E+08 | −1.38347 | 8.23E−06 | AC079135.1; GBX2 |
| chr2_650000_700000 | chr2 | 650000 | 700000 | −1.21475 | 1.76E−09 | AC092159.1; AC092159.3 |
| chr17_73750000_73800000 | chr17 | 73750000 | 73800000 | −1.69298 | 1.69E−05 | AC125421.1 |
| chr4_3750000_3800000 | chr4 | 3750000 | 3800000 | −1.62856 | 2E−12 | AC141928.1; ADRA2C |
| chr17_63450000_63500000 | chr17 | 63450000 | 63500000 | −1.05734 | 2.36E−06 | ACE |
| chr10_133050000_133100000 | chr10 | 1.33E+08 | 1.33E+08 | −1.82195 | 1.49E−09 | ADGRA1; ADGRA1-AS1 |
| chr9_73050000_73100000 | chr9 | 73050000 | 73100000 | 1.79854 | 9.56E−05 | ALDH1A1 |
| chr5_10550000_10600000 | chr5 | 10550000 | 10600000 | −1.07725 | 1.22E−13 | ANKRD33B |
| chr5_10600000_10650000 | chr5 | 10600000 | 10650000 | −1.28289 | 2.53E−22 | ANKRD33B-AS1 |
| chr11_64600000_64650000 | chr11 | 64600000 | 64650000 | −1.00427 | 4.98E−07 | AP001092.4 |
| chr11_64400000_64450000 | chr11 | 64400000 | 64450000 | −1.09774 | 2.49E−11 | AP003774.4 |
| chr11_64350000_64400000 | chr11 | 64350000 | 64400000 | −1.06934 | 1.35E−07 | AP003774.6 |
| chr11_64450000_64500000 | chr11 | 64450000 | 64500000 | −1.42725 | 2.67E−19 | AP005273.1 |
| chr11_64500000_64550000 | chr11 | 64500000 | 64550000 | −1.19624 | 2.47E−21 | AP005273.1 |
| chr17_66250000_66300000 | chr17 | 66250000 | 66300000 | −1.80233 | 2.4E−21 | APOH |
| chr15_58100000_58150000 | chr15 | 58100000 | 58150000 | −1.12337 | 0.0003 | AQP9 |
| chr19_1050000_1100000 | chr19 | 1050000 | 1100000 | −1.21637 | 5.65E−11 | ARHGAP45 |
| chr2_85750000_85800000 | chr2 | 85750000 | 85800000 | −1.20415 | 3.2E−14 | ATOH8 |
| chr13_113650000_113700000 | chr13 | 1.14E+08 | 1.14E+08 | −1.24571 | 2.16E−13 | ATP4B; GRK1 |
| chr17_34550000_34600000 | chr17 | 34550000 | 34600000 | −1.41712 | 4.53E−18 | C17orf102; TMEM132E |
| chr22_49650000_49700000 | chr22 | 49650000 | 49700000 | −1.30684 | 3.93E−11 | C22orf34 |
| chr15_62150000_62200000 | chr15 | 62150000 | 62200000 | −1.29678 | 3.97E−08 | C2CD4B |
| chr19_400000_450000 | chr19 | 400000 | 450000 | −1.03052 | 1.11E−05 | C2CD4C |
| chr17_60150000_60200000 | chr17 | 60150000 | 60200000 | −1.0426 | 2.58E−09 | CA4 |
| chr17_66800000_66850000 | chr17 | 66800000 | 66850000 | −1.20236 | 4.74E−14 | CACNG5 |
| chr16_88950000_89000000 | chr16 | 88950000 | 89000000 | −1.0954 | 9.66E−11 | CBFA2T3; CTD-2555A7.1 |
| chr17_79750000_79800000 | chr17 | 79750000 | 79800000 | −1.64389 | 1.08E−11 | CBX2 |
| chr17_74450000_74500000 | chr17 | 74450000 | 74500000 | −1.71917 | 1.35E−10 | CD300A |
| chr17_74500000_74550000 | chr17 | 74500000 | 74550000 | −1.42622 | 3.24E−06 | CD300LB; CD300C |
| chr2_173350000_173400000 | chr2 | 1.73E+08 | 1.73E+08 | −1.23826 | 0.000236 | CDCA7 |
| chr14_23100000_23150000 | chr14 | 23100000 | 23150000 | −1.0244 | 4.24E−05 | CEBPE |
| chr22_17250000_17300000 | chr22 | 17250000 | 17300000 | −1.05714 | 1.71E−09 | CECR3 |
| chr2_181650000_181700000 | chr2 | 1.82E+08 | 1.82E+08 | −1.15771 | 2.94E−10 | CERKL; NEUROD1; |
| | | | | | | AC013733.3 |
| chr10_132900000_132950000 | chr10 | 1.33E+08 | 1.33E+08 | −1.24943 | 1.75E−12 | CFAP46 |
| chr11_46350000_46400000 | chr11 | 46350000 | 46400000 | −1.44764 | 0.000272 | CHRM4 |
| chr19_14550000_14600000 | chr19 | 14550000 | 14600000 | −1.40803 | 1.01E−05 | CLEC17A |
| chr5_178550000_178600000 | chr5 | 1.79E+08 | 1.79E+08 | −1.04047 | 2.06E−10 | COL23A1 |
| chr19_55350000_55400000 | chr19 | 55350000 | 55400000 | −1.26839 | 4.54E−06 | COX6B2; CTD- |
| | | | | | | 2105E13.6; FAM71E2; |
| | | | | | | IL11; TMEM190 |
| chr18_59300000_59350000 | chr18 | 59300000 | 59350000 | −1.15321 | 6.96E−10 | CPLX4 |
| chr3_194350000_194400000 | chr3 | 1.94E+08 | 1.94E+08 | −1.79826 | 4.24E−18 | CPN2; LRRC15; GP5 |
| chr5_146400000_146450000 | chr5 | 1.46E+08 | 1.46E+08 | −1.03565 | 0.007277 | CTB-1H10.1 |
| chr19_14250000_14300000 | chr19 | 14250000 | 14300000 | −1.30407 | 7.7E−09 | CTD-2189E23.2 |
| chr5_1850000_1900000 | chr5 | 1850000 | 1900000 | −1.23911 | 3.7E−22 | CTD-2194D22.1; CTD- |
| | | | | | | 2194D22.2; CTD- |
| | | | | | | 2194D22.3; IRX4; |
| | | | | | | CTD-2194D22.4 |
| chr5_1550000_1600000 | chr5 | 1550000 | 1600000 | −1.02172 | 3.99E−10 | CTD-2245E15.3; |
| | | | | | | CTD-2012J19.3 |
| chr14_28800000_28850000 | chr14 | 28800000 | 28850000 | 1.652933 | 0.00178 | CTD-2384A14.1 |
| chr17_80450000_80500000 | chr17 | 80450000 | 80500000 | −1.34562 | 6.3E−09 | CTD-2526A2.2; NPTX1 |
| chr11_62350000_62400000 | chr11 | 62350000 | 62400000 | −1.28724 | 1.2E−15 | CTD-2531D15.5 |
| chr17_73700000_73750000 | chr17 | 73700000 | 73750000 | −1.06378 | 6.78E−05 | CTD-2532D12.4 |
| chr19_51300000_51350000 | chr19 | 51300000 | 51350000 | −1.22403 | 0.000558 | CTD-2616J11.16 |
| chr22_37250000_37300000 | chr22 | 37250000 | 37300000 | −1.25707 | 2.3E−11 | CYTH4 |
| chr18_4450000_4500000 | chr18 | 4450000 | 4500000 | 1.359826 | 0.001529 | DLGAP1 |
| chr18_3950000_4000000 | chr18 | 3950000 | 4000000 | 1.123732 | 0.003263 | DLGAP1-AS4 |
| chr5_13900000_13950000 | chr5 | 13900000 | 13950000 | −1.23042 | 1.38E−12 | DNAH5 |
| chr5_38300000_38350000 | chr5 | 38300000 | 38350000 | −1.11404 | 0.000195 | EGFLAM-AS3 |
| chr1_184950000_185000000 | chr1 | 1.85E+08 | 1.85E+08 | 1.068218 | 0.002831 | FAM129A |
| chr2_28950000_29000000 | chr2 | 28950000 | 29000000 | −1.05175 | 0.004387 | FAM179A |
| chr12_13000000_13050000 | chr12 | 13000000 | 13050000 | 1.458347 | 0.000444 | FAM234B |
| chr2_238900000_238950000 | chr2 | 2.39E+08 | 2.39E+08 | −1.32936 | 2.54E−07 | FLJ43879 |
| chr17_77850000_77900000 | chr17 | 77850000 | 77900000 | −1.52592 | 4.75E−09 | FLJ45079 |
| chr5_180600000_180650000 | chr5 | 1.81E+08 | 1.81E+08 | −1.12303 | 5.12E−15 | FLT4 |
| chr11_65900000_65950000 | chr11 | 65900000 | 65950000 | −1.10015 | 1.79E−07 | FOSL1; TSGA10IP |
| chr3_180850000_180900000 | chr3 | 1.81E+08 | 1.81E+08 | −1.06411 | 7.49E−05 | FXR1; CCDC39 |
| chr11_117800000_117850000 | chr11 | 1.18E+08 | 1.18E+08 | −1.17787 | 0.000402 | FXYD2 |
| chr2_170800000_170850000 | chr2 | 1.71E+08 | 1.71E+08 | −1.69452 | 8.74E−23 | GAD1; AC007405.8 |
| chr12_132300000_132350000 | chr12 | 1.32E+08 | 1.32E+08 | −1.0235 | 2.68E−15 | GALNT9; |
| | | | | | | RP13-895J2.7 |
| chr15_45350000_45400000 | chr15 | 45350000 | 45400000 | −1.32196 | 3.43E−09 | GATM-AS1 |
| chr11_58900000_58950000 | chr11 | 58900000 | 58950000 | −1.22539 | 9.49E−06 | GLYATL1 |
| chr5_178950000_179000000 | chr5 | 1.79E+08 | 1.79E+08 | −1.06954 | 0.001943 | GRM6 |
| chr16_72050000_72100000 | chr16 | 72050000 | 72100000 | −1.57126 | 2.2E−09 | HP; HPR |
| chr17_79700000_79750000 | chr17 | 79700000 | 79750000 | −1.15355 | 1E−06 | HP09025; ENPP7 |
| chr22_36750000_36800000 | chr22 | 36750000 | 36800000 | −1.16711 | 2.97E−08 | IFT27 |
| chr19_17800000_17850000 | chr19 | 17800000 | 17850000 | −1.20685 | 9.98E−08 | INSL3; JAK3 |
| chr18_79850000_79900000 | chr18 | 79850000 | 79900000 | −1.48778 | 1.79E−06 | KCNG2 |
| chr11_64250000_64300000 | chr11 | 64250000 | 64300000 | −1.59904 | 1.12E−05 | KCNK4 |
| chr20_63450000_63500000 | chr20 | 63450000 | 63500000 | −1.38066 | 4.01E−08 | KCNQ2; EEF1A2 |
| chr10_133150000_133200000 | chr10 | 1.33E+08 | 1.33E+08 | −1.74858 | 4.16E−07 | KNDC1 |
| chr7_108000000_108050000 | chr7 | 1.08E+08 | 1.08E+08 | 1.876644 | 0.006441 | LAMB1 |
| chr9_135650000_135700000 | chr9 | 1.36E+08 | 1.36E+08 | −1.05928 | 2.22E−14 | LCN9; SOHLH1 |
| chr5_135950000_136000000 | chr5 | 1.36E+08 | 1.36E+08 | −1.00989 | 1.56E−09 | LECT2 |
| chr8_142150000_142200000 | chr8 | 1.42E+08 | 1.42E+08 | −1.5417 | 2.09E−21 | LINC00051 |
| chr17_73800000_73850000 | chr17 | 73800000 | 73850000 | −1.22798 | 0.000278 | LINC00469 |
| chr2_6750000_6800000 | chr2 | 6750000 | 6800000 | −1.42012 | 1.56E−10 | LINC00487 |
| chr13_27950000_28000000 | chr13 | 27950000 | 28000000 | −1.34625 | 1.82E−05 | LINC00543; CDX2; |
| | | | | | | URAD |
| chr20_62750000_62800000 | chr20 | 62750000 | 62800000 | −1.00624 | 3.87E−08 | LINC00659 |
| chr10_42450000_42500000 | chr10 | 42450000 | 42500000 | −1.3965 | 4.31E−09 | LINC00839 |
| chr3_196600000_196650000 | chr3 | 1.97E+08 | 1.97E+08 | −1.02337 | 4.51E−09 | LINC01063; |
| | | | | | | NRROS; PIGX |
| chr10_132950000_133000000 | chr10 | 1.33E+08 | 1.33E+08 | −1.9464 | 7.64E−24 | LINC01167; LINC01166; |
| | | | | | | LINC01168 |
| chr2_3100000_3150000 | chr2 | 3100000 | 3150000 | −1.2032 | 7.29E−13 | LINC01250; |
| | | | | | | AC019118.3 |
| chr2_174300000_174350000 | chr2 | 1.74E+08 | 1.74E+08 | −1.25709 | 0.000886 | LINC01305 |
| chr1_110050000_110100000 | chr1 | 1.1E+08 | 1.1E+08 | −1.02978 | 9.86E−05 | LINC01397 |
| chr20_44450000_44500000 | chr20 | 44450000 | 44500000 | −1.39707 | 2.52E−05 | LINC01620 |
| chr5_150000_200000 | chr5 | 150000 | 200000 | −1.187 | 1.42E−05 | LRRC14B |
| chr11_57150000_57200000 | chr11 | 57150000 | 57200000 | −1.04874 | 0.00089 | LRRC55 |
| chr16_79600000_79650000 | chr16 | 79600000 | 79650000 | −1.10989 | 4.38E−05 | MAF; RP11-70D24.3; |
| | | | | | | RP11-70D24.2 |
| chr17_62800000_62850000 | chr17 | 62800000 | 62850000 | −1.49812 | 1.43E−23 | MARCH10; RP11-453A 12.1 |
| chr13_112850000_112900000 | chr13 | 1.13E+08 | 1.13E+08 | −1.55061 | 4.86E−10 | MCF2L |
| chr13_102650000_102700000 | chr13 | 1.03E+08 | 1.03E+08 | −1.20503 | 1.9E−06 | METTL21C |
| chr7_4950000_5000000 | chr7 | 4950000 | 5000000 | −1.0104 | 1.16E−05 | MMD2 |
| chr11_60750000_60800000 | chr11 | 60750000 | 60800000 | −1.09898 | 2.27E−18 | MS4A15; MS4A10 |
| chr11_112950000_113000000 | chr11 | 1.13E+08 | 1.13E+08 | −1.02568 | 8.93E−05 | NCAM1; RP11-629G13.1 |
| chrX_71900000_71950000 | chrX | 71900000 | 71950000 | −1.24642 | 0.007701 | NHSL2 |
| chr10_132750000_132800000 | chr10 | 1.33E+08 | 1.33E+08 | −1.05534 | 0.000272 | NKX6-2 |
| chr9_35950000_36000000 | chr9 | 35950000 | 36000000 | 1.212461 | 0.000236 | OR2S2 |
| chr2_19350000_19400000 | chr2 | 19350000 | 19400000 | −1.28904 | 0.000356 | OSR1 |
| chr14_56800000_56850000 | chr14 | 56800000 | 56850000 | −1.06298 | 1.38E−11 | OTX2; OTX2-AS1 |
| chr8_12950000_13000000 | chr8 | 12950000 | 13000000 | −1.13467 | 0.000499 | PEBP4 |
| chr8_13200000_13250000 | chr8 | 13200000 | 13250000 | −1.16954 | 2.48E−05 | PEBP4 |
| chr8_17650000_17700000 | chr8 | 17650000 | 17700000 | −2.69986 | 1.27E−15 | PEBP4 |
| chr8_18450000_18500000 | chr8 | 18450000 | 18500000 | −1.01063 | 0.004993 | PEBP4 |
| chr8_350000_400000 | chr8 | 350000 | 400000 | −1.00967 | 1.48E−07 | PEBP4; FAM87A |
| chr8_18200000_18250000 | chr8 | 18200000 | 18250000 | −1.02744 | 4.14E−05 | PEBP4; PEBP4 |
| chr8_13000000_13050000 | chr8 | 13000000 | 13050000 | −1.32631 | 1.66E−09 | PEBP4; PEBP4 |
| chr8_13150000_13200000 | chr8 | 13150000 | 13200000 | −1.51665 | 2.39E−11 | PEBP4; PEBP4 |
| chr8_17600000_17650000 | chr8 | 17600000 | 17650000 | −1.3765 | 1.82E−07 | PEBP4; PEBP4 |
| chr1_950000_1000000 | chr1 | 950000 | 1000000 | −1.27896 | 5.57E−06 | PERM1; RP11-54O7.17 |
| chr10_6100000_6150000 | chr10 | 6100000 | 6150000 | −1.18925 | 2.49E−15 | PFKFB3 |
| chr20_45900000_45950000 | chr20 | 45900000 | 45950000 | −1.08245 | 0.000326 | PLTP |
| chr19_46450000_46500000 | chr19 | 46450000 | 46500000 | −1.03211 | 5.86E−07 | PNMAL1 |
| chr13_113150000_113200000 | chr13 | 1.13E+08 | 1.13E+08 | −1.02328 | 7.78E−07 | PROZ; RP11-98F14.11 |
| chr19_3750000_3800000 | chr19 | 3750000 | 3800000 | −1.22621 | 8.57E−11 | RAX2 |
| chr12_94250000_94300000 | chr12 | 94250000 | 94300000 | −1.16006 | 6.94E−06 | RP11-1105G2.4; |
| | | | | | | RP11-1105G2.3 |
| chr15_71550000_71600000 | chr15 | 71550000 | 71600000 | −1.01141 | 0.000701 | RP11-1123I8.1 |
| chr15_71500000_71550000 | chr15 | 71500000 | 71550000 | −1.02351 | 0.000396 | RP11-1123I8.1 |
| chr11_67850000_67900000 | chr11 | 67850000 | 67900000 | −1.01713 | 5.59E−06 | RP11-119D9.1 |
| chr5_3450000_3500000 | chr5 | 3450000 | 3500000 | −1.47992 | 1.93E−17 | RP11-121L11.3 |
| chr4_8050000_8100000 | chr4 | 8050000 | 8100000 | −1.30741 | 3.53E−10 | RP11-1258F18.1 |
| chr7_101300000_101350000 | chr7 | 1.01E+08 | 1.01E+08 | −1.45343 | 3.92E−07 | RP11-132A1.3 |
| chr12_118050000_118100000 | chr12 | 1.18E+08 | 1.18E+08 | −1.03919 | 0.001267 | RP11-136L23.2 |
| chr17_62700000_62750000 | chr17 | 62700000 | 62750000 | −1.0654 | 3.03E−19 | RP11-156L14.1 |
| chr2_178700000_178750000 | chr2 | 1.79E+08 | 1.79E+08 | −1.16136 | 9.08E−19 | RP11-171I2.1 |
| chr2_178600000_178650000 | chr2 | 1.79E+08 | 1.79E+08 | −1.16943 | 3.21E−14 | RP11-171I2.4; |
| | | | | | | RP11-171I2.3 |
| chr2_178550000_178600000 | chr2 | 1.79E+08 | 1.79E+08 | −1.46985 | 1.27E−21 | RP11-171I2.5; |
| | | | | | | RP11-171I2.2 |
| chr5_10500000_10550000 | chr5 | 10500000 | 10550000 | −1.00634 | 1.08E−15 | RP11-1C1.4; RP11-1C1.5 |
| chr1_3300000_3350000 | chr1 | 3300000 | 3350000 | −1.04875 | 0.003272 | RP11-22L13.1 |
| chr1_6700000_6750000 | chr1 | 6700000 | 6750000 | −1.53151 | 2.95E−08 | RP11-242F24.1 |
| chr20_63350000_63400000 | chr20 | 63350000 | 63400000 | −1.02252 | 9.88E−13 | RP11-261N11.8; CHRNA4 |
| chr8_52150000_52200000 | chr8 | 52150000 | 52200000 | −1.0021 | 1.28E−09 | RP11-26M5.3; |
| | | | | | | RP11-26M5.2 |
| chr4_185450000_185500000 | chr4 | 1.85E+08 | 1.86E+08 | 2.380619 | 0.001999 | RP11-279O9.4; CCDC110 |
| chr15_62200000_62250000 | chr15 | 62200000 | 62250000 | −1.20134 | 5.65E−07 | RP11-299H22.1 |
| chr15_62250000_62300000 | chr15 | 62250000 | 62300000 | −1.7386 | 1.62E−10 | RP11-299H22.7 |
| chr1_7000000_7050000 | chr1 | 7000000 | 7050000 | −1.07579 | 1.29E−07 | RP11-334N17.1 |
| chr4_8000000_8050000 | chr4 | 8000000 | 8050000 | −1.03029 | 1.71E−09 | RP11-338K13.1 |
| chr17_79100000_79150000 | chr17 | 79100000 | 79150000 | −1.16282 | 1.16E−07 | RP11-398J5.1 |
| chr15_62350000_62400000 | chr15 | 62350000 | 62400000 | −1.14322 | 2.82E−11 | RP11-435J9.2; TLN2 |
| chr18_77050000_77100000 | chr18 | 77050000 | 77100000 | −3.23317 | 1.48E−17 | RP11-4B16.3 |
| chr6_3300000_3350000 | chr6 | 3300000 | 3350000 | −1.93831 | 2.09E−21 | RP11-506K6.4 |
| chr1_1050000_1100000 | chr1 | 1050000 | 1100000 | −1.05732 | 2.03E−05 | RP11-54O7.18; RNF223 |
| chr14_56500000_56550000 | chr14 | 56500000 | 56550000 | −1.13952 | 1.73E−05 | RP11-624J12.1 |
| chr17_74300000_74350000 | chr17 | 74300000 | 74350000 | −1.05469 | 7.04E−08 | RP11-647F2.2; KIF19 |
| chr5_179500000_179550000 | chr5 | 1.8E+08 | 1.8E+08 | −1.10803 | 0.000165 | RP11-798K23.4 |
| chr12_131350000_131400000 | chr12 | 1.31E+08 | 1.31E+08 | −1.72051 | 2.35E−11 | RP13-507P19.1 |
| chr12_132250000_132300000 | chr12 | 1.32E+08 | 1.32E+08 | −1.23833 | 2.22E−11 | RP13-895J2.2; |
| | | | | | | RP13-895J2.3; |
| | | | | | | RP13-895J2.6 |
| chr4_3600000_3650000 | chr4 | 3600000 | 3650000 | −1.08324 | 2.9E−06 | RP3-368B9.2 |
| chr4_3650000_3700000 | chr4 | 3650000 | 3700000 | −1.35836 | 5.1E−12 | RP3-513G18.2 |
| chr6_1000000_1050000 | chr6 | 1000000 | 1050000 | −1.11331 | 1.23E−12 | RP5-856G1.1 |
| chr2_199450000_199500000 | chr2 | 1.99E+08 | 2E+08 | −1.11263 | 0.00013 | SATB2- AS1; SATB2 |
| chr11_62400000_62450000 | chr11 | 62400000 | 62450000 | −1.50124 | 6.07E−15 | SCGB1A1; CTD-2531D15.4 |
| chr22_30500000_30550000 | chr22 | 30500000 | 30550000 | −1.38728 | 0.000595 | SEC14L4 |
| chr16_4950000_5000000 | chr16 | 4950000 | 5000000 | −1.20974 | 9.91E−11 | SEC14L5; PPL |
| chr14_36500000_36550000 | chr14 | 36500000 | 36550000 | −1.05782 | 2.72E−06 | SFTA3; NKX2-1-AS1; |
| | | | | | | NKX2-1 |
| chr12_111400000_111450000 | chr12 | 1.11E+08 | 1.11E+08 | −1.04798 | 0.000323 | SH2B3 |
| chr19_450000_500000 | chr19 | 450000 | 500000 | −1.12505 | 0.000277 | SHC2; ODF3L2; |
| | | | | | | MADCAM1 |
| chr19_51450000_51500000 | chr19 | 51450000 | 51500000 | −2.03959 | 7.67E−07 | SIGLEC8 |
| chr11_64550000_64600000 | chr11 | 64550000 | 64600000 | −1.12216 | 3.5E−15 | SLC22A11; |
| | | | | | | SLC22A12 |
| chr6_3450000_3500000 | chr6 | 3450000 | 3500000 | −1.0949 | 1.05E−11 | SLC22A23 |
| chr16_89200000_89250000 | chr16 | 89200000 | 89250000 | −1.27616 | 0.000247 | SLC22A31 |
| chr19_6400000_6450000 | chr19 | 6400000 | 6450000 | −1.08148 | 6.78E−05 | SLC25A41 |
| chr11_57500000_57550000 | chr11 | 57500000 | 57550000 | −1.00379 | 1.12E−06 | SLC43A1; SMTNL1 |
| chr19_17850000_17900000 | chr19 | 17850000 | 17900000 | −1.08249 | 4.02E−05 | SLC5A5 |
| chr5_1200000_1250000 | chr5 | 1200000 | 1250000 | −1.3662 | 1.82E−22 | SLC6A19; SLC6A18 |
| chr3_165150000_165200000 | chr3 | 1.65E+08 | 1.65E+08 | −1.28057 | 0.00177 | SLITRK3 |
| chr11_130900000_130950000 | chr11 | 1.31E+08 | 1.31E+08 | −1.00601 | 0.007277 | SNX19 |
| chr1_153050000_153100000 | chr1 | 1.53E+08 | 1.53E+08 | 1.39812 | 0.000882 | SPRR2A |
| chr1_153100000_153150000 | chr1 | 1.53E+08 | 1.53E+08 | 1.523265 | 0.000427 | SPRR2G |
| chr12_10650000_10700000 | chr12 | 10650000 | 10700000 | 1.621621 | 0.004887 | STYK1 |
| chr19_11350000_11400000 | chr19 | 11350000 | 11400000 | −1.0463 | 6.46E−06 | SWSAP1; CTD- |
| | | | | | | 2342J14.6; EPOR |
| chr11_67600000_67650000 | chr11 | 67600000 | 67650000 | −1.35543 | 0.000272 | TBX10 |
| chr17_61350000_61400000 | chr17 | 61350000 | 61400000 | −1.00431 | 1.52E−06 | TBX2 |
| chr5_1250000_1300000 | chr5 | 1250000 | 1300000 | −1.10092 | 3.07E−11 | TERT |
| chr6_50800000_50850000 | chr6 | 50800000 | 50850000 | 1.560261 | 5.75E−05 | TFAP2B |
| chr6_158500000_158550000 | chr6 | 1.59E+08 | 1.59E+08 | −1.01298 | 1.52E−06 | TMEM181 |
| chr17_10700000_10750000 | chr17 | 10700000 | 10750000 | −1.00513 | 7.26E−07 | TMEM220-AS1; |
| | | | | | | TMEM220 |
| chr1_175050000_175100000 | chr1 | 1.75E+08 | 1.75E+08 | −1.03928 | 1.06E−15 | TNN |
| chr2_238800000_238850000 | chr2 | 2.39E+08 | 2.39E+08 | −1.14262 | 3.26E−08 | TWIST2 |
| chr10_117100000_117150000 | chr10 | 1.17E+08 | 1.17E+08 | −1.29555 | 6.11E−06 | VAX1 |
| chr2_174650000_174700000 | chr2 | 1.75E+08 | 1.75E+08 | −1.22565 | 6.59E−07 | WIPF1 |
| chr16_17450000_17500000 | chr16 | 17450000 | 17500000 | −1.44538 | 1.59E−09 | XYLT1 |
| chr22_43250000_43300000 | chr22 | 43250000 | 43300000 | −1.28094 | 2.27E−08 | Z99756.1 |
| chr16_2800000_2850000 | chr16 | 2800000 | 2850000 | −1.40171 | 0.00039 | ZG16B |
| chr19_44050000_44100000 | chr19 | 44050000 | 44100000 | −1.04168 | 0.000426 | ZNF223 |
| chr16_49850000_49900000 | chr16 | 49850000 | 49900000 | −1.15524 | 5.49E−07 | ZNF423 |
| chr19_5450000_5500000 | chr19 | 5450000 | 5500000 | −1.04759 | 2.29E−09 | ZNRF4 |

TABLE 2
genomic regions of depleted in H3K27me3 enrichment in resistant and resistant-like
cells compared to sensitive cells for Capecitabine treatment, non-overlapping a
transcription start site, q-value < 0.01 (sixth column) and |log2FC| > 1 (fifth
column). The first column relates to the identification numbers of the genomic
sequences using the reference genome GRCh38 (GeneBank assembly accession
GCA_000001405.15), second column shows the chromosome number. The third and
fourth columns relate to the start and end position of genomic sequence respectively.

| | | | | log2FC. res. | adj. |
|---|---|---|---|---|---|
| ID | chr | Start | end | vs. sens | pval |
| chr1_184500000_184550000 | chr1 | 1.85E+08 | 1.85E+08 | 1.703358 | 0.002603 |
| chr1_7300000_7350000 | chr1 | 7300000 | 7350000 | −2.00093 | 2.22E−18 |
| chr1_216000000_216050000 | chr1 | 2.16E+08 | 2.16E+08 | 1.550191 | 0.003777 |
| chr1_4450000_4500000 | chr1 | 4450000 | 4500000 | −1.01717 | 0.002299 |
| chr1_6000000_6050000 | chr1 | 6000000 | 6050000 | −1.02909 | 8.66E−09 |
| chr1_7050000_7100000 | chr1 | 7050000 | 7100000 | −1.58679 | 4.84E−12 |
| chr1_7200000_7250000 | chr1 | 7200000 | 7250000 | −1.37604 | 2.12E−12 |
| chr1_7600000_7650000 | chr1 | 7600000 | 7650000 | −1.13246 | 6.8E−09 |
| chr1_18700000_18750000 | chr1 | 18700000 | 18750000 | −1.00934 | 3.31E−08 |
| chr1_34950000_35000000 | chr1 | 34950000 | 35000000 | −1.12938 | 4.16E−06 |
| chr1_85300000_85350000 | chr1 | 85300000 | 85350000 | −1.31095 | 0.001747 |
| chr1_178750000_178800000 | chr1 | 1.79E+08 | 1.79E+08 | 1.813959 | 0.00066 |
| chr1_185950000_186000000 | chr1 | 1.86E+08 | 1.86E+08 | 1.132933 | 0.008953 |
| chr1_222950000_223000000 | chr1 | 2.23E+08 | 2.23E+08 | 1.591981 | 0.008527 |
| chr1_233000000_233050000 | chr1 | 2.33E+08 | 2.33E+08 | 1.463604 | 0.008718 |
| chr1_241650000_241700000 | chr1 | 2.42E+08 | 2.42E+08 | 1.573111 | 5.07E−05 |
| chr1_241800000_241850000 | chr1 | 2.42E+08 | 2.42E+08 | 1.680636 | 0.006678 |
| chr1_3200000_3250000 | chr1 | 3200000 | 3250000 | −1.51054 | 3.87E−10 |
| chr1_4200000_4250000 | chr1 | 4200000 | 4250000 | −1.23488 | 0.000161 |
| chr1_5300000_5350000 | chr1 | 5300000 | 5350000 | −1.21185 | 0.002253 |
| chr1_7100000_7150000 | chr1 | 7100000 | 7150000 | −1.32785 | 1.97E−09 |
| chr1_7150000_7200000 | chr1 | 7150000 | 7200000 | −1.40459 | 1.97E−08 |
| chr1_7250000_7300000 | chr1 | 7250000 | 7300000 | −1.97673 | 1.63E−24 |
| chr1_7350000_7400000 | chr1 | 7350000 | 7400000 | −2.55441 | 1.35E−10 |
| chr1_7500000_7550000 | chr1 | 7500000 | 7550000 | −1.52105 | 6.01E−13 |
| chr1_7550000_7600000 | chr1 | 7550000 | 7600000 | −2.14612 | 1.62E−13 |
| chr1_11300000_11350000 | chr1 | 11300000 | 11350000 | −1.04955 | 0.000282 |
| chr1_25500000_25550000 | chr1 | 25500000 | 25550000 | −1.21853 | 0.002057 |
| chr1_65150000_65200000 | chr1 | 65150000 | 65200000 | −1.01781 | 8.35E−14 |
| chr1_97800000_97850000 | chr1 | 97800000 | 97850000 | 1.293935 | 0.00123 |
| chr1_110550000_110600000 | chr1 | 1.11E+08 | 1.11E+08 | 1.464861 | 0.001936 |
| chr1_154350000_154400000 | chr1 | 1.54E+08 | 1.54E+08 | −1.03691 | 6.81E−16 |
| chr1_171850000_171900000 | chr1 | 1.72E+08 | 1.72E+08 | 1.110073 | 0.006107 |
| chr1_178000000_178050000 | chr1 | 1.78E+08 | 1.78E+08 | 1.584452 | 0.00186 |
| chr1_178700000_178750000 | chr1 | 1.79E+08 | 1.79E+08 | 1.691778 | 0.000283 |
| chr1_184850000_184900000 | chr1 | 1.85E+08 | 1.85E+08 | 1.086434 | 0.00575 |
| chr1_185800000_185850000 | chr1 | 1.86E+08 | 1.86E+08 | 1.231087 | 1.64E−05 |
| chr1_199300000_199350000 | chr1 | 1.99E+08 | 1.99E+08 | 2.408183 | 0.002773 |
| chr1_207150000_207200000 | chr1 | 2.07E+08 | 2.07E+08 | 2.080851 | 0.003895 |
| chr1_215650000_215700000 | chr1 | 2.16E+08 | 2.16E+08 | 1.714067 | 0.004897 |
| chr1_216200000_216250000 | chr1 | 2.16E+08 | 2.16E+08 | 2.540948 | 0.000174 |
| chr1_216700000_216750000 | chr1 | 2.17E+08 | 2.17E+08 | 1.632707 | 0.002003 |
| chr1_222900000_222950000 | chr1 | 2.23E+08 | 2.23E+08 | 2.053532 | 0.006573 |
| chr1_244200000_244250000 | chr1 | 2.44E+08 | 2.44E+08 | −1.13229 | 2.62E−19 |
| chr10_132100000_132150000 | chr10 | 1.32E+08 | 1.32E+08 | −2.58882 | 1.47E−10 |
| chr10_1550000_1600000 | chr10 | 1550000 | 1600000 | −1.0994 | 2.22E−06 |
| chr10_3650000_3700000 | chr10 | 3650000 | 3700000 | 1.694478 | 0.004443 |
| chr10_12800000_12850000 | chr10 | 12800000 | 12850000 | −1.06619 | 0.000105 |
| chr10_12950000_13000000 | chr10 | 12950000 | 13000000 | −1.6436 | 2.75E−21 |
| chr10_22250000_22300000 | chr10 | 22250000 | 22300000 | −1.23274 | 0.000446 |
| chr10_23700000_23750000 | chr10 | 23700000 | 23750000 | −1.06389 | 0.002556 |
| chr10_45600000_45650000 | chr10 | 45600000 | 45650000 | −1.09931 | 3.31E−08 |
| chr10_85550000_85600000 | chr10 | 85550000 | 85600000 | −1.01603 | 0.004844 |
| chr10_126200000_126250000 | chr10 | 1.26E+08 | 1.26E+08 | −1.23226 | 6E−06 |
| chr10_128050000_128100000 | chr10 | 1.28E+08 | 1.28E+08 | −1.5342 | 7.12E−08 |
| chr10_131550000_131600000 | chr10 | 1.32E+08 | 1.32E+08 | −1.2437 | 0.006307 |
| chr10_131700000_131750000 | chr10 | 1.32E+08 | 1.32E+08 | −1.45384 | 4.75E−05 |
| chr10_4350000_4400000 | chr10 | 4350000 | 4400000 | 1.738394 | 0.002829 |
| chr10_12850000_12900000 | chr10 | 12850000 | 12900000 | −1.0578 | 2.16E−08 |
| chr10_23600000_23650000 | chr10 | 23600000 | 23650000 | −1.0418 | 0.0007 |
| chr10_29950000_30000000 | chr10 | 29950000 | 30000000 | −1.03345 | 0.003763 |
| chr10_38150000_38200000 | chr10 | 38150000 | 38200000 | −1.33036 | 5.02E−05 |
| chr10_126300000_126350000 | chr10 | 1.26E+08 | 1.26E+08 | −1.08201 | 0.001362 |
| chr10_132850000_132900000 | chr10 | 1.33E+08 | 1.33E+08 | −1.08342 | 9.63E−15 |
| chr10_133100000_133150000 | chr10 | 1.33E+08 | 1.33E+08 | −1.20201 | 1.57E−07 |
| chr11_8350000_8400000 | chr11 | 8350000 | 8400000 | −1.04715 | 1.94E−05 |
| chr11_60800000_60850000 | chr11 | 60800000 | 60850000 | −1.04425 | 3.8E−07 |
| chr11_116350000_116400000 | chr11 | 1.76E+08 | 1.16E+08 | −1.48571 | 0.003783 |
| chr11_1400000_1450000 | chr11 | 1400000 | 1450000 | −1.92964 | 1.44E−11 |
| chr11_3500000_3550000 | chr11 | 3500000 | 3550000 | −1.11685 | 1.04E−07 |
| chr11_19250000_19300000 | chr11 | 19250000 | 19300000 | −1.03649 | 0.003568 |
| chr11_44400000_44450000 | chr11 | 44400000 | 44450000 | −1.10481 | 2.57E−08 |
| chr11_61600000_61650000 | chr11 | 61600000 | 61650000 | −1.08585 | 0.001333 |
| chr11_61700000_61750000 | chr11 | 61700000 | 61750000 | −1.28899 | 1.41E−05 |
| chr11_109550000_109600000 | chr11 | 1.25E+08 | 1.1E+08 | −1.18028 | 0.002851 |
| chr11_113100000_113150000 | chr11 | 1.13E+08 | 1.13E+08 | −1.31631 | 9.96E−05 |
| chr11_115600000_115650000 | chr11 | 1.16E+08 | 1.16E+08 | −1.51953 | 0.000417 |
| chr11_124900000_124950000 | chr11 | 1.25E+08 | 1.25E+08 | −1.76914 | 4.91E−07 |
| chr11_125300000_125350000 | chr11 | 1.25E+08 | 1.25E+08 | −1.09307 | 0.001802 |
| chr11_125400000_125450000 | chr11 | 1.25E+08 | 1.25E+08 | −1.08324 | 1.03E−05 |
| chr11_134300000_134350000 | chr11 | 1.34E+08 | 1.34E+08 | −1.26225 | 0.0002 |
| chr11_134600000_134650000 | chr11 | 1.35E+08 | 1.35E+08 | −1.14713 | 0.008687 |
| chr11_1450000_1500000 | chr11 | 1450000 | 1500000 | −1.40029 | 3.22E−07 |
| chr11_5300000_5350000 | chr11 | 5300000 | 5350000 | −1.57528 | 0.001303 |
| chr11_35850000_35900000 | chr11 | 35850000 | 35900000 | −1.00448 | 0.007054 |
| chr11_44350000_44400000 | chr11 | 44350000 | 44400000 | −1.10257 | 1.4E−07 |
| chr11_61000000_61050000 | chr11 | 61000000 | 61050000 | −1.59334 | 4.27E−06 |
| chr11_64950000_65000000 | chr11 | 64950000 | 65000000 | −1.028 | 0.001667 |
| chr11_65300000_65350000 | chr11 | 65300000 | 65350000 | −1.0665 | 0.000254 |
| chr11_83300000_83350000 | chr11 | 83300000 | 83350000 | −1.27672 | 4.45E−11 |
| chr11_90200000_90250000 | chr11 | 90200000 | 90250000 | 2.048584 | 0.0011 |
| chr11_109100000_109150000 | chr11 | 1.09E+08 | 1.09E+08 | −1.82979 | 0.007431 |
| chr11_112500000_112550000 | chr11 | 1.13E+08 | 1.13E+08 | −1.274 | 4.36E−11 |
| chr11_112900000_112950000 | chr11 | 1.13E+08 | 1.13E+08 | −1.11797 | 5.69E−08 |
| chr11_114100000_114150000 | chr11 | 1.14E+08 | 1.14E+08 | −1.12348 | 1.34E−09 |
| chr11_114900000_114950000 | chr11 | 1.15E+08 | 1.15E+08 | −1.4021 | 0.000109 |
| chr11_115650000_115700000 | chr11 | 1.16E+08 | 1.16E+08 | −1.79415 | 0.000527 |
| chr11_125200000_125250000 | chr11 | 1.25E+08 | 1.25E+08 | −1.03398 | 0.001161 |
| chr11_125350000_125400000 | chr11 | 1.25E+08 | 1.25E+08 | −1.30388 | 8.02E−05 |
| chr11_134500000_134550000 | chr11 | 1.35E+08 | 1.35E+08 | −1.62529 | 2.14E−06 |
| chr12_10050000_10100000 | chr12 | 10050000 | 10100000 | 1.435649 | 0.000395 |
| chr12_54750000_54800000 | chr12 | 54750000 | 54800000 | −1.0467 | 4.81E−05 |
| chr12_5400000_5450000 | chr12 | 5400000 | 5450000 | −1.16176 | 0.003205 |
| chr12_9250000_9300000 | chr12 | 9250000 | 9300000 | 1.150544 | 0.000794 |
| chr12_13700000_13750000 | chr12 | 13700000 | 13750000 | 1.119335 | 0.000899 |
| chr12_26000000_26050000 | chr12 | 26000000 | 26050000 | 2.320188 | 0.004025 |
| chr12_34100000_34150000 | chr12 | 34100000 | 34150000 | −1.23192 | 0.006238 |
| chr12_34250000_34300000 | chr12 | 34250000 | 34300000 | −1.14028 | 2.12E−07 |
| chr12_38150000_38200000 | chr12 | 38150000 | 38200000 | −1.02888 | 4.84E−05 |
| chr12_110050000_110100000 | chr12 | 1.1E+08 | 1.1E+08 | −1.26057 | 0.000427 |
| chr12_130450000_130500000 | chr12 | 1.3E+08 | 1.31E+08 | −1.12974 | 0.000306 |
| chr12_131650000_131700000 | chr12 | 1.32E+08 | 1.32E+08 | −1.01189 | 2.03E−06 |
| chr12_132950000_133000000 | chr12 | 1.33E+08 | 1.33E+08 | −1.62212 | 0.003763 |
| chr13_25950000_26000000 | chr13 | 25950000 | 26000000 | −1.10289 | 0.006407 |
| chr13_44650000_44700000 | chr13 | 44650000 | 44700000 | −1.12856 | 0.001796 |
| chr13_110200000_110250000 | chr13 | 1.1E+08 | 1.1E+08 | −1.74674 | 1.19E−22 |
| chr13_110350000_110400000 | chr13 | 1.1E+08 | 1.1E+08 | −1.57715 | 6.32E−22 |
| chr13_28350000_28400000 | chr13 | 28350000 | 28400000 | −1.09035 | 1.38E−06 |
| chr13_28500000_28550000 | chr13 | 28500000 | 28550000 | −1.05731 | 0.001624 |
| chr13_99900000_99950000 | chr13 | 99900000 | 99950000 | −1.2164 | 2.9E−06 |
| chr13_100800000_100850000 | chr13 | 1.01E+08 | 1.01E+08 | −1.717 | 0.000598 |
| chr13_110150000_110200000 | chr13 | 1.1E+08 | 1.1E+08 | −1.73291 | 3.7E−22 |
| chr13_110250000_110300000 | chr13 | 1.1E+08 | 1.1E+08 | −1.55496 | 3.96E−17 |
| chr13_110400000_110450000 | chr13 | 1.1E+08 | 1.1E+08 | −1.27041 | 2.35E−16 |
| chr13_112650000_112700000 | chr13 | 1.13E+08 | 1.13E+08 | −1.33497 | 6.34E−10 |
| chr13_113950000_114000000 | chr13 | 1.14E+08 | 1.14E+08 | −1.17094 | 0.002559 |
| chr14_52000000_52050000 | chr14 | 52000000 | 52050000 | −1.24241 | 0.0014 |
| chr14_63550000_63600000 | chr14 | 63550000 | 63600000 | −1.45904 | 0.000289 |
| chr14_81000000_81050000 | chr14 | 81000000 | 81050000 | −1.0042 | 6.24E−06 |
| chr14_81100000_81150000 | chr14 | 81100000 | 81150000 | −1.51256 | 2.84E−05 |
| chr14_59150000_59200000 | chr14 | 59150000 | 59200000 | 2.183487 | 0.009181 |
| chr14_65800000_65850000 | chr14 | 65800000 | 65850000 | −1.55194 | 0.000696 |
| chr15_70900000_70950000 | chr15 | 70900000 | 70950000 | 2.131749 | 0.000141 |
| chr15_87900000_87950000 | chr15 | 87900000 | 87950000 | −1.67374 | 5.55E−05 |
| chr15_88200000_88250000 | chr15 | 88200000 | 88250000 | −1.06051 | 6.9E−05 |
| chr15_94200000_94250000 | chr15 | 94200000 | 94250000 | −1.28591 | 0.005182 |
| chr15_98400000_98450000 | chr15 | 98400000 | 98450000 | −1.4489 | 5.32E−09 |
| chr15_23650000_23700000 | chr15 | 23650000 | 23700000 | −1.23327 | 0.007732 |
| chr15_33350000_33400000 | chr15 | 33350000 | 33400000 | 2.4234 | 0.00089 |
| chr15_71100000_71150000 | chr15 | 71100000 | 71150000 | −1.25789 | 2.87E−05 |
| chr15_72250000_72300000 | chr15 | 72250000 | 72300000 | −1.67028 | 4.54E−05 |
| chr15_87600000_87650000 | chr15 | 87600000 | 87650000 | −1.17403 | 0.000619 |
| chr15_88000000_88050000 | chr15 | 88000000 | 88050000 | −1.30016 | 2.9E−05 |
| chr16_6150000_6200000 | chr16 | 6150000 | 6200000 | −1.61516 | 0.000817 |
| chr16_11250000_11300000 | chr16 | 11250000 | 11300000 | −1.54771 | 1.62E−06 |
| chr16_12950000_13000000 | chr16 | 12950000 | 13000000 | −1.3094 | 0.003354 |
| chr16_20050000_20100000 | chr16 | 20050000 | 20100000 | −1.13282 | 0.000458 |
| chr16_48100000_48150000 | chr16 | 48100000 | 48150000 | −1.15822 | 4.95E−09 |
| chr16_61700000_61750000 | chr16 | 61700000 | 61750000 | −1.13654 | 0.000358 |
| chr16_77700000_77750000 | chr16 | 77700000 | 77750000 | −1.42812 | 0.001383 |
| chr16_78900000_78950000 | chr16 | 78900000 | 78950000 | −1.02144 | 0.000719 |
| chr16_86100000_86150000 | chr16 | 86100000 | 86150000 | −1.38214 | 0.000145 |
| chr16_3950000_4000000 | chr16 | 3950000 | 4000000 | −1.08116 | 0.000443 |
| chr16_10250000_10300000 | chr16 | 10250000 | 10300000 | 1.649916 | 0.00274 |
| chr16_12650000_12700000 | chr16 | 12650000 | 12700000 | −1.10151 | 0.00053 |
| chr16_13000000_13050000 | chr16 | 13000000 | 13050000 | −1.15069 | 0.00274 |
| chr16_17500000_17550000 | chr16 | 17500000 | 17550000 | −2.13436 | 6.18E−08 |
| chr16_49050000_49100000 | chr16 | 49050000 | 49100000 | −1.73151 | 0.000711 |
| chr16_64650000_64700000 | chr16 | 64650000 | 64700000 | −1.77506 | 0.002257 |
| chr16_65550000_65600000 | chr16 | 65550000 | 65600000 | −1.2921 | 0.000293 |
| chr16_65850000_65900000 | chr16 | 65850000 | 65900000 | −1.25892 | 3.54E−05 |
| chr16_79700000_79750000 | chr16 | 79700000 | 79750000 | −1.22851 | 0.002816 |
| chr16_79750000_79800000 | chr16 | 79750000 | 79800000 | −1.50527 | 0.001384 |
| chr16_83150000_83200000 | chr16 | 83150000 | 83200000 | −1.54855 | 4.4E−05 |
| chr16_84300000_84350000 | chr16 | 84300000 | 84350000 | −1.27849 | 5.76E−13 |
| chr17_34600000_34650000 | chr17 | 34600000 | 34650000 | −1.27973 | 1.24E−08 |
| chr17_62900000_62950000 | chr17 | 62900000 | 62950000 | −1.02139 | 6.55E−14 |
| chr17_6650000_6700000 | chr17 | 6650000 | 6700000 | −1.03826 | 1.4E−11 |
| chr17_34450000_34500000 | chr17 | 34450000 | 34500000 | −1.22912 | 4.57E−05 |
| chr17_60400000_60450000 | chr17 | 60400000 | 60450000 | −1.0055 | 4.93E−08 |
| chr17_72600000_72650000 | chr17 | 72600000 | 72650000 | −1.00165 | 0.006324 |
| chr17_4650000_4700000 | chr17 | 4650000 | 4700000 | −1.19444 | 0.003867 |
| chr17_11350000_11400000 | chr17 | 11350000 | 11400000 | −1.33153 | 0.000122 |
| chr17_21950000_22000000 | chr17 | 21950000 | 22000000 | −1.03613 | 7.19E−13 |
| chr17_34500000_34550000 | chr17 | 34500000 | 34550000 | −1.02708 | 9.76E−07 |
| chr17_56700000_56750000 | chr17 | 56700000 | 56750000 | −1.01296 | 7.64E−24 |
| chr17_65150000_65200000 | chr17 | 65150000 | 65200000 | −1.06337 | 5.11E−16 |
| chr17_66200000_66250000 | chr17 | 66200000 | 66250000 | −1.43575 | 2.25E−17 |
| chr17_73550000_73600000 | chr17 | 73550000 | 73600000 | −1.1232 | 1.09E−09 |
| chr17_73950000_74000000 | chr17 | 73950000 | 74000000 | −1.49284 | 2.07E−05 |
| chr17_74000000_74050000 | chr17 | 74000000 | 74050000 | −1.35127 | 1.1E−07 |
| chr17_77600000_77650000 | chr17 | 77600000 | 77650000 | −1.00989 | 2.7E−09 |
| chr17_79050000_79100000 | chr17 | 79050000 | 79100000 | −1.27032 | 5.6E−06 |
| chr17_79300000_79350000 | chr17 | 79300000 | 79350000 | −1.09248 | 5.98E−10 |
| chr18_1550000_1600000 | chr18 | 1550000 | 1600000 | 1.648921 | 0.000142 |
| chr18_2200000_2250000 | chr18 | 2200000 | 2250000 | 1.701009 | 0.002571 |
| chr18_3650000_3700000 | chr18 | 3650000 | 3700000 | 1.951961 | 1.17E−05 |
| chr18_3800000_3850000 | chr18 | 3800000 | 3850000 | 1.497146 | 2.03E−05 |
| chr18_4550000_4600000 | chr18 | 4550000 | 4600000 | 1.477665 | 0.008031 |
| chr18_35900000_35950000 | chr18 | 35900000 | 35950000 | 1.714657 | 0.001837 |
| chr18_36400000_36450000 | chr18 | 36400000 | 36450000 | 2.128372 | 0.006743 |
| chr18_51200000_51250000 | chr18 | 51200000 | 51250000 | −1.03904 | 1.91E−08 |
| chr18_1050000_1100000 | chr18 | 1050000 | 1100000 | 1.552271 | 0.001943 |
| chr18_1600000_1650000 | chr18 | 1600000 | 1650000 | 1.955026 | 0.000138 |
| chr18_3200000_3250000 | chr18 | 3200000 | 3250000 | 1.878371 | 0.000432 |
| chr18_3700000_3750000 | chr18 | 3700000 | 3750000 | 1.422112 | 4.71E−10 |
| chr18_3750000_3800000 | chr18 | 3750000 | 3800000 | 1.195597 | 5.31E−06 |
| chr18_4000000_4050000 | chr18 | 4000000 | 4050000 | 1.162231 | 8.17E−05 |
| chr18_4350000_4400000 | chr18 | 4350000 | 4400000 | 1.664361 | 0.000326 |
| chr18_4600000_4650000 | chr18 | 4600000 | 4650000 | 1.838991 | 0.006171 |
| chr18_25300000_25350000 | chr18 | 25300000 | 25350000 | 1.527804 | 0.001297 |
| chr18_31650000_31700000 | chr18 | 31650000 | 31700000 | 4.167086 | 0.000392 |
| chr18_33700000_33750000 | chr18 | 33700000 | 33750000 | 2.484546 | 0.000691 |
| chr18_36650000_36700000 | chr18 | 36650000 | 36700000 | 1.799654 | 0.00644 |
| chr18_60200000_60250000 | chr18 | 60200000 | 60250000 | −1.22311 | 4.93E−06 |
| chr18_76050000_76100000 | chr18 | 76050000 | 76100000 | −1.06078 | 0.005921 |
| chr18_77000000_77050000 | chr18 | 77000000 | 77050000 | −1.77965 | 3.7E−21 |
| chr18_78400000_78450000 | chr18 | 78400000 | 78450000 | −1.58652 | 5.14E−05 |
| chr18_49250000_49300000 | chr19 | 49250000 | 49300000 | −1.14055 | 4.99E−06 |
| chr19_51350000_51400000 | chr19 | 51350000 | 51400000 | −1.00106 | 0.001936 |
| chr19_6650000_6700000 | chr19 | 6650000 | 6700000 | −1.03981 | 0.000544 |
| chr19_13200000_13250000 | chr19 | 13200000 | 13250000 | −1.32198 | 1.58E−07 |
| chr19_54000000_54050000 | chr19 | 54000000 | 54050000 | −1.19239 | 6.8E−07 |
| chr19_2650000_2700000 | chr19 | 2650000 | 2700000 | −1.04632 | 1.12E−08 |
| chr19_3150000_3200000 | chr19 | 3150000 | 3200000 | −1.18475 | 0.004966 |
| chr19_28200000_28250000 | chr19 | 28200000 | 28250000 | −1.0437 | 2.52E−08 |
| chr19_35050000_35100000 | chr19 | 35050000 | 35100000 | −1.12944 | 0.000368 |
| chr19_38450000_38500000 | chr19 | 38450000 | 38500000 | −1.01216 | 8.21E−06 |
| chr19_39850000_39900000 | chr19 | 39850000 | 39900000 | −1.25048 | 0.001208 |
| chr2_23050000_23100000 | chr2 | 23050000 | 23100000 | −1.0355 | 4.21E−05 |
| chr2_40850000_40900000 | chr2 | 40850000 | 40900000 | −1.38671 | 0.004404 |
| chr2_85950000_86000000 | chr2 | 85950000 | 86000000 | −1.54056 | 1.27E−11 |
| chr2_233800000_233850000 | chr2 | 2.34E+08 | 2.34E+08 | −1.20206 | 3.28E−06 |
| chr2_2450000_2500000 | chr2 | 2450000 | 2500000 | −1.49472 | 0.001872 |
| chr2_2850000_2900000 | chr2 | 2850000 | 2900000 | −1.0184 | 0.003231 |
| chr2_6650000_6700000 | chr2 | 6650000 | 6700000 | −1.25761 | 0.000161 |
| chr2_6800000_6850000 | chr2 | 6800000 | 6850000 | −1.34613 | 3.63E−11 |
| chr2_12150000_12200000 | chr2 | 12150000 | 12200000 | −1.16528 | 0.00492 |
| chr2_22400000_22450000 | chr2 | 22400000 | 22450000 | −1.35154 | 6.09E−05 |
| chr2_26850000_26900000 | chr2 | 26850000 | 26900000 | −1.05961 | 3.47E−13 |
| chr2_64450000_64500000 | chr2 | 64450000 | 64500000 | −1.17654 | 0.008767 |
| chr2_84100000_84150000 | chr2 | 84100000 | 84150000 | −1.07598 | 0.001162 |
| chr2_100250000_100300000 | chr2 | 1E+08 | 1E+08 | 2.150171 | 0.000209 |
| chr2_104950000_105000000 | chr2 | 1.05E+08 | 1.05E+08 | 1.28313 | 0.004832 |
| chr2_117700000_117750000 | chr2 | 1.18E+08 | 1.18E+08 | 1.754579 | 0.00624 |
| chr2_118050000_118100000 | chr2 | 1.18E+08 | 1.18E+08 | 1.9142 | 0.000869 |
| chr2_176400000_176450000 | chr2 | 1.76E+08 | 1.76E+08 | −1.37353 | 0.001505 |
| chr2_177050000_177100000 | chr2 | 1.77E+08 | 1.77E+08 | −1.0333 | 2.55E−11 |
| chr2_178650000_178700000 | chr2 | 1.79E+08 | 1.79E+08 | −1.05572 | 1.32E−09 |
| chr2_50000_100000 | chr2 | 50000 | 100000 | −1.17498 | 1.6E−06 |
| chr2_1650000_1700000 | chr2 | 1650000 | 1700000 | −1.18883 | 6.99E−05 |
| chr2_1850000_1900000 | chr2 | 1850000 | 1900000 | −1.11104 | 4.86E−05 |
| chr2_2000000_2050000 | chr2 | 2000000 | 2050000 | −1.50164 | 8.45E−07 |
| chr2_2150000_2200000 | chr2 | 2150000 | 2200000 | −1.40181 | 0.000167 |
| chr2_2250000_2300000 | chr2 | 2250000 | 2300000 | −1.17994 | 0.000259 |
| chr2_2900000_2950000 | chr2 | 2900000 | 2950000 | −1.0003 | 0.004784 |
| chr2_3600000_3650000 | chr2 | 3600000 | 3650000 | −1.88031 | 2.01E−15 |
| chr2_3950000_4000000 | chr2 | 3950000 | 4000000 | −1.05685 | 0.002895 |
| chr2_10500000_10550000 | chr2 | 10500000 | 10550000 | −1.02047 | 4.81E−06 |
| chr2_11850000_11900000 | chr2 | 11850000 | 11900000 | −1.25014 | 9.48E−09 |
| chr2_12000000_12050000 | chr2 | 12000000 | 12050000 | −1.08777 | 4.15E−12 |
| chr2_21250000_21300000 | chr2 | 21250000 | 21300000 | −1.02728 | 1.47E−05 |
| chr2_21550000_21600000 | chr2 | 21550000 | 21600000 | −1.13311 | 0.0011 |
| chr2_23400000_23450000 | chr2 | 23400000 | 23450000 | −1.19267 | 2.02E−07 |
| chr2_25100000_25150000 | chr2 | 25100000 | 25150000 | −1.08045 | 8.35E−14 |
| chr2_27550000_27600000 | chr2 | 27550000 | 27600000 | −1.13349 | 2.86E−05 |
| chr2_61800000_61850000 | chr2 | 61800000 | 61850000 | −1.13915 | 0.00774 |
| chr2_64900000_64950000 | chr2 | 64900000 | 64950000 | −1.01044 | 4.96E−07 |
| chr2_65300000_65350000 | chr2 | 65300000 | 65350000 | −1.01504 | 0.000213 |
| chr2_73300000_73350000 | chr2 | 73300000 | 73350000 | −1.29104 | 8.84E−12 |
| chr2_85900000_85950000 | chr2 | 85900000 | 85950000 | −1.02651 | 4.18E−07 |
| chr2_88250000_88300000 | chr2 | 88250000 | 88300000 | −1.19037 | 5.85E−06 |
| chr2_170850000_170900000 | chr2 | 1.71E+08 | 1.71E+08 | −1.18008 | 1.03E−10 |
| chr2_172150000_172200000 | chr2 | 1.72E+08 | 1.72E+08 | −1.18227 | 1.38E−06 |
| chr2_173750000_173800000 | chr2 | 1.74E+08 | 1.74E+08 | −1.03036 | 0.001813 |
| chr2_174750000_174800000 | chr2 | 1.75E+08 | 1.75E+08 | −1.17873 | 0.000443 |
| chr2_176200000_176250000 | chr2 | 1.76E+08 | 1.76E+08 | −1.36586 | 6.34E−10 |
| chr2_177000000_177050000 | chr2 | 1.77E+08 | 1.77E+08 | −1.08851 | 4.04E−09 |
| chr2_177650000_177700000 | chr2 | 1.78E+08 | 1.78E+08 | −1.05552 | 1.14E−06 |
| chr2_180000000_180050000 | chr2 | 1.8E+08 | 1.8E+08 | −1.288 | 0.001387 |
| chr2_192850000_192900000 | chr2 | 1.93E+08 | 1.93E+08 | −1.56664 | 4.36E−12 |
| chr2_200050000_200100000 | chr2 | 2E+08 | 2E+08 | −1.20132 | 0.000546 |
| chr2_200200000_200250000 | chr2 | 2E+08 | 2E+08 | −1.71996 | 9.63E−06 |
| chr2_206450000_206500000 | chr2 | 2.06E+08 | 2.07E+08 | −1.01927 | 0.005164 |
| chr2_236200000_236250000 | chr2 | 2.36E+08 | 2.36E+08 | −1.22637 | 1.77E−08 |
| chr2_238700000_238750000 | chr2 | 2.39E+08 | 2.39E+08 | −1.14448 | 7.01E−05 |
| chr20_61850000_61900000 | chr20 | 61850000 | 61900000 | −1.23278 | 2.51E−11 |
| chr20_22150000_22200000 | chr20 | 22150000 | 22200000 | −1.06731 | 2.38E−05 |
| chr20_29050000_29100000 | chr20 | 29050000 | 29100000 | 1.984034 | 9.34E−05 |
| chr20_31050000_31100000 | chr20 | 31050000 | 31100000 | −1.01872 | 5.32E−15 |
| chr20_48350000_48400000 | chr20 | 48350000 | 48400000 | −1.00631 | 0.000665 |
| chr20_62100000_62150000 | chr20 | 62100000 | 62150000 | −1.00493 | 0.000483 |
| chr20_63400000_63450000 | chr20 | 63400000 | 63450000 | −1.60551 | 4.27E−07 |
| chr20_29250000_29300000 | chr20 | 29250000 | 29300000 | 2.201218 | 1.82E−07 |
| chr20_29300000_29350000 | chr20 | 29300000 | 29350000 | 1.029045 | 0.009307 |
| chr20_43150000_43200000 | chr20 | 43150000 | 43200000 | −1.19405 | 0.009125 |
| chr20_43950000_44000000 | chr20 | 43950000 | 44000000 | −1.20597 | 0.00048 |
| chr20_44250000_44300000 | chr20 | 44250000 | 44300000 | −1.1374 | 4.44E−06 |
| chr20_48300000_48350000 | chr20 | 48300000 | 48350000 | −1.49426 | 3.61E−10 |
| chr20_48550000_48600000 | chr20 | 48550000 | 48600000 | −1.21247 | 1.91E−06 |
| chr20_60450000_60500000 | chr20 | 60450000 | 60500000 | −1.19108 | 3.47E−05 |
| chr20_60600000_60650000 | chr20 | 60600000 | 60650000 | −1.01331 | 0.001472 |
| chr20_61150000_61200000 | chr20 | 61150000 | 61200000 | −1.00715 | 0.000543 |
| chr20_61350000_61400000 | chr20 | 61350000 | 61400000 | −1.2252 | 6.77E−07 |
| chr20_62050000_62100000 | chr20 | 62050000 | 62100000 | −1.01514 | 1.2E−06 |
| chr20_62950000_63000000 | chr20 | 62950000 | 63000000 | −1.10242 | 7.25E−13 |
| chr20_63600000_63650000 | chr20 | 63600000 | 63650000 | −1.28666 | 8.72E−05 |
| chr20_63750000_63800000 | chr20 | 63750000 | 63800000 | −1.24282 | 3.35E−14 |
| chr21_32500000_32550000 | chr21 | 32500000 | 32550000 | −1.40444 | 0.006637 |
| chr21_32600000_32650000 | chr21 | 32600000 | 32650000 | −1.04684 | 0.004505 |
| chr21_41500000_41550000 | chr21 | 41500000 | 41550000 | −1.22552 | 0.000546 |
| chr21_42900000_42950000 | chr21 | 42900000 | 42950000 | −1.13447 | 0.002586 |
| chr22_47150000_47200000 | chr22 | 47150000 | 47200000 | −1.00364 | 0.006991 |
| chr22_49250000_49300000 | chr22 | 49250000 | 49300000 | −1.05503 | 0.003111 |
| chr22_39700000_39750000 | chr22 | 39700000 | 39750000 | −1.06677 | 8.97E−08 |
| chr22_48350000_48400000 | chr22 | 48350000 | 48400000 | −1.6512 | 0.005326 |
| chr22_48600000_48650000 | chr22 | 48600000 | 48650000 | −1.34723 | 0.000498 |
| chr22_25300000_25350000 | chr22 | 25300000 | 25350000 | −1.17411 | 0.001708 |
| chr22_27050000_27100000 | chr22 | 27050000 | 27100000 | −1.5259 | 2.05E−05 |
| chr22_39600000_39650000 | chr22 | 39600000 | 39650000 | −1.02697 | 8.16E−08 |
| chr22_39650000_39700000 | chr22 | 39650000 | 39700000 | −1.0308 | 0.00167 |
| chr22_47250000_47300000 | chr22 | 47250000 | 47300000 | −1.10449 | 1.43E−06 |
| chr22_48500000_48550000 | chr22 | 48500000 | 48550000 | −1.22377 | 0.000741 |
| chr22_48650000_48700000 | chr22 | 48650000 | 48700000 | −1.39759 | 0.001096 |
| chr22_49600000_49650000 | chr22 | 49600000 | 49650000 | −1.40128 | 5.79E−10 |
| chr3_184500000_184550000 | chr3 | 1.85E+08 | 1.85E+08 | −1.15206 | 3.17E−05 |
| chr3_10450000_10500000 | chr3 | 10450000 | 10500000 | −1.0227 | 0.002066 |
| chr3_50950000_51000000 | chr3 | 50950000 | 51000000 | 2.113869 | 0.00522 |
| chr3_139300000_139350000 | chr3 | 1.39E+08 | 1.39E+08 | −1.04011 | 8.7E−05 |
| chr3_194600000_194650000 | chr3 | 1.95E+08 | 1.95E+08 | −1.37636 | 0.000435 |
| chr3_196200000_196250000 | chr3 | 1.96E+08 | 1.96E+08 | −1.33663 | 7.66E−05 |
| chr3_4600000_4650000 | chr3 | 4600000 | 4650000 | −1.0859 | 2.03E−10 |
| chr3_4650000_4700000 | chr3 | 4650000 | 4700000 | −1.55173 | 5.57E−25 |
| chr3_10300000_10350000 | chr3 | 10300000 | 10350000 | −1.11643 | 6.67E−05 |
| chr3_11150000_11200000 | chr3 | 11150000 | 11200000 | −1.09439 | 0.001214 |
| chr3_64550000_64600000 | chr3 | 64550000 | 64600000 | 2.449593 | 0.004463 |
| chr3_72300000_72350000 | chr3 | 72300000 | 72350000 | −1.19329 | 0.004934 |
| chr3_154700000_154750000 | chr3 | 1.55E+08 | 1.55E+08 | 1.569021 | 0.003982 |
| chr3_180800000_180850000 | chr3 | 1.81E+08 | 1.81E+08 | −1.06799 | 3.05E−05 |
| chr3_181800000_181850000 | chr3 | 1.82E+08 | 1.82E+08 | −1.08333 | 0.0045 |
| chr3_192950000_193000000 | chr3 | 1.93E+08 | 1.93E+08 | −1.25858 | 0.008334 |
| chr3_194300000_194350000 | chr3 | 1.94E+08 | 1.94E+08 | −1.49923 | 2.62E−06 |
| chr3_195900000_195950000 | chr3 | 1.96E+08 | 1.96E+08 | −1.35024 | 1.65E−05 |
| chr3_196650000_196700000 | chr3 | 1.97E+08 | 1.97E+08 | −1.32667 | 7.19E−13 |
| chr4_3800000_3850000 | chr4 | 3800000 | 3850000 | −1.12542 | 2.72E−07 |
| chr4_4700000_4750000 | chr4 | 4700000 | 4750000 | −1.16068 | 5.01E−06 |
| chr4_6400000_6450000 | chr4 | 6400000 | 6450000 | −1.10966 | 3.37E−11 |
| chr4_7200000_7250000 | chr4 | 7200000 | 7250000 | −1.44176 | 0.000254 |
| chr4_7600000_7650000 | chr4 | 7600000 | 7650000 | −1.11559 | 3.31E−07 |
| chr4_8100000_8150000 | chr4 | 8100000 | 8150000 | −1.27345 | 3.93E−10 |
| chr4_37050000_37100000 | chr4 | 37050000 | 37100000 | 1.622976 | 0.004623 |
| chr4_186350000_186400000 | chr4 | 1.86E+08 | 1.86E+08 | −2.0491 | 3.02E−09 |
| chr4_3550000_3600000 | chr4 | 3550000 | 3600000 | −1.48579 | 5.69E−06 |
| chr4_3700000_3750000 | chr4 | 3700000 | 3750000 | −1.15261 | 8.14E−09 |
| chr4_7250000_7300000 | chr4 | 7250000 | 7300000 | −1.76235 | 1.11E−13 |
| chr4_7350000_7400000 | chr4 | 7350000 | 7400000 | −1.01509 | 4.34E−10 |
| chr4_7500000_7550000 | chr4 | 7500000 | 7550000 | −1.17743 | 3.64E−10 |
| chr4_7650000_7700000 | chr4 | 7650000 | 7700000 | −1.38695 | 2.23E−11 |
| chr4_33200000_33250000 | chr4 | 33200000 | 33250000 | −1.63638 | 0.000119 |
| chr4_89850000_89900000 | chr4 | 89850000 | 89900000 | 1.815783 | 0.000178 |
| chr4_139750000_139800000 | chr4 | 1.4E+08 | 1.4E+08 | −1.09146 | 7.48E−09 |
| chr4_151850000_151900000 | chr4 | 1.52E+08 | 1.52E+08 | −1.12118 | 0.005857 |
| chr4_177000000_177050000 | chr4 | 1.77E+08 | 1.77E+08 | −1.34652 | 0.002008 |
| chr5_178250000_178300000 | chr5 | 1.78E+08 | 1.78E+08 | −1.16648 | 1.71E−08 |
| chr5_100000_150000 | chr5 | 100000 | 150000 | −1.03017 | 0.004544 |
| chr5_750000_800000 | chr5 | 750000 | 800000 | −1.51693 | 4.87E−09 |
| chr5_2200000_2250000 | chr5 | 2200000 | 2250000 | −1.21818 | 2.85E−06 |
| chr5_3400000_3450000 | chr5 | 3400000 | 3450000 | −1.00955 | 3.79E−11 |
| chr5_3650000_3700000 | chr5 | 3650000 | 3700000 | −1.56024 | 8.7E−16 |
| chr5_3900000_3950000 | chr5 | 3900000 | 3950000 | −1.16976 | 2.83E−11 |
| chr5_4050000_4100000 | chr5 | 4050000 | 4100000 | −1.04937 | 9E−12 |
| chr5_13200000_13250000 | chr5 | 13200000 | 13250000 | −1.35939 | 0.00027 |
| chr5_13500000_13550000 | chr5 | 13500000 | 13550000 | −1.04017 | 0.000393 |
| chr5_22900000_22950000 | chr5 | 22900000 | 22950000 | −1.12921 | 0.004527 |
| chr5_38100000_38150000 | chr5 | 38100000 | 38150000 | −1.15777 | 2.4E−05 |
| chr5_154500000_154550000 | chr5 | 1.55E+08 | 1.55E+08 | −1.21072 | 9.24E−12 |
| chr5_159250000_159300000 | chr5 | 1.59E+08 | 1.59E+08 | −1.18739 | 0.000281 |
| chr5_176650000_176700000 | chr5 | 1.77E+08 | 1.77E+08 | −1.12364 | 3.6E−11 |
| chr5_178500000_178550000 | chr5 | 1.79E+08 | 1.79E+08 | −1.97241 | 3.22E−18 |
| chr5_1500000_1550000 | chr5 | 1500000 | 1550000 | −1.14583 | 3.95E−08 |
| chr5_1600000_1650000 | chr5 | 1600000 | 1650000 | −1.19874 | 1.75E−12 |
| chr5_3300000_3350000 | chr5 | 3300000 | 3350000 | −1.08313 | 4.48E−14 |
| chr5_3350000_3400000 | chr5 | 3350000 | 3400000 | −1.06478 | 5.33E−14 |
| chr5_3600000_3650000 | chr5 | 3600000 | 3650000 | −1.7006 | 3.03E−19 |
| chr5_4200000_4250000 | chr5 | 4200000 | 4250000 | −1.52483 | 2.94E−15 |
| chr5_5800000_5850000 | chr5 | 5800000 | 5850000 | −1.0873 | 4.73E−07 |
| chr5_6100000_6150000 | chr5 | 6100000 | 6150000 | −1.05096 | 0.004505 |
| chr5_8700000_8750000 | chr5 | 8700000 | 8750000 | −1.18242 | 0.000279 |
| chr5_11050000_11100000 | chr5 | 11050000 | 11100000 | −1.11464 | 0.000106 |
| chr5_15750000_15800000 | chr5 | 15750000 | 15800000 | −1.06629 | 0.000113 |
| chr5_165700000_165750000 | chr5 | 1.66E+08 | 1.66E+08 | −1.18654 | 0.000175 |
| chr5_173150000_173200000 | chr5 | 1.73E+08 | 1.73E+08 | −1.09528 | 6.64E−06 |
| chr5_178800000_178850000 | chr5 | 1.79E+08 | 1.79E+08 | −1.07791 | 1.24E−08 |
| chr6_13200000_13250000 | chr6 | 13200000 | 13250000 | −1.28978 | 5.09E−15 |
| chr6_15850000_15900000 | chr6 | 15850000 | 15900000 | −1.06729 | 0.000215 |
| chr6_54000000_54050000 | chr6 | 54000000 | 54050000 | 1.588479 | 0.009233 |
| chr6_166450000_166500000 | chr6 | 1.66E+08 | 1.67E+08 | −1.14912 | 1.79E−06 |
| chr6_170000000_170050000 | chr6 | 1.7E+08 | 1.7E+08 | −1.65293 | 0.000213 |
| chr6_12500000_12550000 | chr6 | 12500000 | 12550000 | −1.0061 | 9.21E−08 |
| chr6_18250000_18300000 | chr6 | 18250000 | 18300000 | −1.27297 | 0.001503 |
| chr6_35800000_35850000 | chr6 | 35800000 | 35850000 | −1.13461 | 4.31E−05 |
| chr6_42150000_42200000 | chr6 | 42150000 | 42200000 | −1.1289 | 0.001805 |
| chr6_151550000_151600000 | chr6 | 1.52E+08 | 1.52E+08 | −1.26859 | 9.9E−07 |
| chr6_168500000_168550000 | chr6 | 1.69E+08 | 1.69E+08 | −1.02766 | 0.000701 |
| chr7_30650000_30700000 | chr7 | 30650000 | 30700000 | −1.03112 | 0.00682 |
| chr7_45450000_45500000 | chr7 | 45450000 | 45500000 | −1.08517 | 0.002406 |
| chr7_72200000_72250000 | chr7 | 72200000 | 72250000 | −1.19661 | 0.000381 |
| chr7_75750000_75800000 | chr7 | 75750000 | 75800000 | −1.02541 | 0.000474 |
| chr7_115450000_115500000 | chr7 | 1.15E+08 | 1.16E+08 | 1.806006 | 0.006802 |
| chr7_121100000_121150000 | chr7 | 1.21E+08 | 1.21E+08 | 2.524725 | 0.000277 |
| chr7_145600000_145650000 | chr7 | 1.46E+08 | 1.46E+08 | −1.04935 | 0.000698 |
| chr7_150850000_150900000 | chr7 | 1.51E+08 | 1.51E+08 | −1.38629 | 7.33E−11 |
| chr7_151350000_151400000 | chr7 | 1.51E+08 | 1.51E+08 | −1.14288 | 1.94E−06 |
| chr7_153450000_153500000 | chr7 | 1.53E+08 | 1.54E+08 | −1.10439 | 0.00121 |
| chr7_156600000_156650000 | chr7 | 1.57E+08 | 1.57E+08 | 1.819824 | 0.005656 |
| chr7_158050000_158100000 | chr7 | 1.58E+08 | 1.58E+08 | −2.02991 | 4.45E−06 |
| chr7_5300000_5350000 | chr7 | 5300000 | 5350000 | −1.08253 | 0.001663 |
| chr7_11200000_11250000 | chr7 | 11200000 | 11250000 | 1.934273 | 0.002902 |
| chr7_11350000_11400000 | chr7 | 11350000 | 11400000 | 2.358751 | 0.002017 |
| chr7_13950000_14000000 | chr7 | 13950000 | 14000000 | 3.153483 | 1.92E−06 |
| chr7_19700000_19750000 | chr7 | 19700000 | 19750000 | 1.43247 | 2.17E−05 |
| chr7_19950000_20000000 | chr7 | 19950000 | 20000000 | 2.840691 | 0.003355 |
| chr7_21050000_21100000 | chr7 | 21050000 | 21100000 | 1.664889 | 0.000138 |
| chr7_23350000_23400000 | chr7 | 23350000 | 23400000 | −1.01139 | 0.001837 |
| chr7_23400000_23450000 | chr7 | 23400000 | 23450000 | −2.10411 | 3.01E−13 |
| chr7_28750000_28800000 | chr7 | 28750000 | 28800000 | 1.307975 | 0.008415 |
| chr7_31200000_31250000 | chr7 | 31200000 | 31250000 | −1.41899 | 1.55E−10 |
| chr7_52600000_52650000 | chr7 | 52600000 | 52650000 | −1.08516 | 0.003871 |
| chr7_53650000_53700000 | chr7 | 53650000 | 53700000 | −1.08681 | 0.000982 |
| chr7_67550000_67600000 | chr7 | 67550000 | 67600000 | −1.40304 | 0.000413 |
| chr7_71750000_71800000 | chr7 | 71750000 | 71800000 | −2.01811 | 8.03E−06 |
| chr7_72500000_72550000 | chr7 | 72500000 | 72550000 | −1.04025 | 3.81E−09 |
| chr7_74200000_74250000 | chr7 | 74200000 | 74250000 | −1.30875 | 1.71E−05 |
| chr7_93400000_93450000 | chr7 | 93400000 | 93450000 | 1.743687 | 0.004412 |
| chr7_103350000_103400000 | chr7 | 1.03E+08 | 1.03E+08 | −1.0392 | 0.00103 |
| chr8_141550000_141600000 | chr8 | 1.42E+08 | 1.42E+08 | −1.52835 | 6.6E−18 |
| chr8_20050000_20100000 | chr8 | 20050000 | 20100000 | −1.49899 | 0.001959 |
| chr8_21600000_21650000 | chr8 | 21600000 | 21650000 | −1.05779 | 0.009543 |
| chr8_107600000_107650000 | chr8 | 1.08E+08 | 1.08E+08 | 2.243707 | 0.007406 |
| chr8_16700000_16750000 | chr8 | 16700000 | 16750000 | −1.07862 | 0.0014 |
| chr8_24000000_24050000 | chr8 | 24000000 | 24050000 | −1.07184 | 0.000892 |
| chr8_36850000_36900000 | chr8 | 36850000 | 36900000 | 2.099794 | 0.00024 |
| chr8_52350000_52400000 | chr8 | 52350000 | 52400000 | −1.01734 | 2.24E−11 |
| chr8_103750000_103800000 | chr8 | 1.04E+08 | 1.04E+08 | 2.040187 | 0.003554 |
| chr8_107750000_107800000 | chr8 | 1.08E+08 | 1.08E+08 | 1.209063 | 0.006783 |
| chr8_141600000_141650000 | chr8 | 1.42E+08 | 1.42E+08 | −1.69111 | 2.47E−21 |
| chr8_141700000_141750000 | chr8 | 1.42E+08 | 1.42E+08 | −1.15716 | 1.53E−12 |
| chr8_142100000_142150000 | chr8 | 1.42E+08 | 1.42E+08 | −1.03843 | 1.05E−11 |
| chr9_111100000_111150000 | chr9 | 1.11E+08 | 1.11E+08 | 1.943764 | 0.00529 |
| chr9_9300000_9350000 | chr9 | 9300000 | 9350000 | 1.942168 | 0.003212 |
| chr9_21750000_21800000 | chr9 | 21750000 | 21800000 | 2.238915 | 2.01E−05 |
| chr9_76000000_76050000 | chr9 | 76000000 | 76050000 | 1.373577 | 0.00168 |
| chr9_95000000_95050000 | chr9 | 95000000 | 95050000 | 2.122202 | 0.000161 |
| chr9_9100000_9150000 | chr9 | 9100000 | 9150000 | 3.191778 | 3.68E−05 |
| chr9_9750000_9800000 | chr9 | 9750000 | 9800000 | 1.966968 | 0.000427 |
| chr9_16200000_16250000 | chr9 | 16200000 | 16250000 | −1.60019 | 1.63E−17 |
| chr9_20350000_20400000 | chr9 | 20350000 | 20400000 | 2.067641 | 0.002934 |
| chr9_20450000_20500000 | chr9 | 20450000 | 20500000 | 2.284938 | 5.55E−05 |
| chr9_21550000_21600000 | chr9 | 21550000 | 21600000 | 2.112609 | 6.07E−07 |
| chr9_22300000_22350000 | chr9 | 22300000 | 22350000 | 2.140563 | 0.00438 |
| chr9_36050000_36100000 | chr9 | 36050000 | 36100000 | 1.547258 | 2.2E−06 |
| chr9_41200000_41250000 | chr9 | 41200000 | 41250000 | 2.579796 | 1.6E−06 |
| chr9_69500000_69550000 | chr9 | 69500000 | 69550000 | 2.138678 | 0.000364 |
| chr9_69900000_69950000 | chr9 | 69900000 | 69950000 | 1.746124 | 0.001376 |
| chr9_72950000_73000000 | chr9 | 72950000 | 73000000 | 1.69976 | 0.002934 |
| chr9_76100000_76150000 | chr9 | 76100000 | 76150000 | 1.212597 | 0.005139 |
| chr9_78950000_79000000 | chr9 | 78950000 | 79000000 | 1.971806 | 0.006881 |
| chr9_103300000_103350000 | chr9 | 1.03E+08 | 1.03E+08 | 1.867707 | 0.003835 |
| chr9_110950000_111000000 | chr9 | 1.11E+08 | 1.11E+08 | 1.39514 | 0.008662 |
| chr9_115650000_115700000 | chr9 | 1.16E+08 | 1.16E+08 | 2.423535 | 8.22E−05 |
| chr9_133350000_133400000 | chr9 | 1.33E+08 | 1.33E+08 | −1.14483 | 0.002273 |
| chr9_134150000_134200000 | chr9 | 1.34E+08 | 1.34E+08 | −1.10697 | 6.54E−06 |
| chr9_134750000_134800000 | chr9 | 1.35E+08 | 1.35E+08 | −1.01851 | 1.01E−05 |
| chr9_135800000_135850000 | chr9 | 1.36E+08 | 1.36E+08 | −1.22168 | 1.9E−06 |
| chr9_137250000_137300000 | chr9 | 1.37E+08 | 1.37E+08 | −1.16105 | 0.000293 |
| chrX_8650000_8700000 | chrX | 8650000 | 8700000 | −1.14376 | 0.00022 |
| chrX_9300000_9350000 | chrX | 9300000 | 9350000 | −1.23728 | 2.31E−10 |
| chrX_9800000_9850000 | chrX | 9800000 | 9850000 | −1.20961 | 4.1E−05 |
| chrX_48600000_48650000 | chrX | 48600000 | 48650000 | −1.31485 | 5.25E−05 |
| chrX_48850000_48900000 | chrX | 48850000 | 48900000 | −1.61975 | 4.86E−06 |
| chrX_50450000_50500000 | chrX | 50450000 | 50500000 | −1.01133 | 0.006974 |
| chrX_4750000_4800000 | chrX | 4750000 | 4800000 | −1.27214 | 0.001767 |
| chrX_8550000_8600000 | chrX | 8550000 | 8600000 | −1.2716 | 3.01E−07 |
| chrX_9350000_9400000 | chrX | 9350000 | 9400000 | −1.33188 | 4.38E−06 |
| chrX_9900000_9950000 | chrX | 9900000 | 9950000 | −1.87787 | 4.48E−14 |
| chrX_49750000_49800000 | chrX | 49750000 | 49800000 | −1.51656 | 0.001208 |

TABLE 3
genomic regions depleted in H3K27me3 enrichment in resistant and resistant-like cells compared to
sensitive cells for Capecitabine treatment overlapping a transcription start site of a gene known
to be associated to resistance to chemotherapy, q-value < 0.01 (sixth column) and |log2FC| >
1 (fifth column). The first column relates to the identification numbers of the genomic sequences
using the reference genome GRCh38 (GeneBank assembly accession GCA_000001405.15), second column
shows the chromosome number. The third and fourth columns relate to the start and end position of
genomic sequence respectively. Last column indicates genes related to transcription start site.

| | | | | log2FC. res. | adj. | Associated |
|---|---|---|---|---|---|---|
| ID | chr | start | end | vs. sens | pval | Gene |
| chr13_110300000_110350000 | chr13 | 1.1E+08 | 1.1E+08 | −1.25823 | 7.75E−16 | COL4A2; COL4A1 |
| chr2_176100000_176150000 | chr2 | 1.76E+08 | 1.76E+08 | −1.81279 | 2.78E−16 | HOXD10; HOXD9; |
| | | | | | | HOXD8; HOXD3; |
| | | | | | | HOXD-AS2 |
| chr7_23450000_23500000 | chr7 | 23450000 | 23500000 | −2.21142 | 5.84E−20 | IGF2BP3 |

TABLE 4
genomic regions depleted in H3K27me3 enrichment in resistant or resistant-like cell compared to sensitive
cells for Tamoxifen treatment overlapping a transcription start site, q-value < 0.01 (sixth column)
and |log2FC| > 1 (fifth column). The first column relates to the identification numbers of the
genomic sequences using the reference genome GRCh38 (GeneBank assembly accession GCA_000001405.15),
second column shows the chromosome number. The third and fourth columns relate to the start and end
position of genomic sequence respectively. Last column indicates genes related to transcription start site.

| | | | | log2FC. res. | adj. | Associated |
|---|---|---|---|---|---|---|
| ID | chr | start | end | vs. sens | pval | Gene |
| chr16_26550000_26600000 | chr16 | 26550000 | 26600000 | 2.282175836 | 0.006723134 | AC002331.1 |
| chr7_157600000_157650000 | chr7 | 157600000 | 157650000 | −2.546526344 | 7.84763E−13 | AC005481.5 |
| chr7_157500000_157550000 | chr7 | 157500000 | 157550000 | −1.757776423 | 3.19602E−05 | AC006372.6 |
| chr7_155450000_155500000 | chr7 | 155450000 | 155500000 | −2.396651037 | 4.32309E−05 | AC008060.8; |
| | | | | | | EN2 |
| chr7_158000000_158050000 | chr7 | 158000000 | 158050000 | −1.540182428 | 0.000249232 | AC011899.10 |
| chr7_157850000_157900000 | chr7 | 157850000 | 157900000 | −1.953305259 | 0.001644975 | AC011899.9; |
| | | | | | | RP11-452C13.1 |
| chr2_104650000_104700000 | chr2 | 104650000 | 104700000 | −1.41488221 | 0.000132514 | AC013402.5 |
| chr2_104700000_104750000 | chr2 | 104700000 | 104750000 | −1.44004599 | 4.59956E−05 | AC068057.1 |
| chr7_1250000_1300000 | chr7 | 1250000 | 1300000 | −1.947412701 | 0.00659844 | AC073094.4 |
| chr2_1050000_1100000 | chr2 | 1050000 | 1100000 | −2.569367433 | 0.008810734 | AC114808.3 |
| chr13_111100000_111150000 | chr13 | 111100000 | 111150000 | −1.370898901 | 3.50405E−05 | ARHGEF7; |
| | | | | | | ARHGEF7-AS2; |
| | | | | | | ARHGEF7-AS1 |
| chr2_60550000_60600000 | chr2 | 60550000 | 60600000 | −1.855898293 | 0.006770829 | BCL11A |
| chr22_49650000_49700000 | chr22 | 49650000 | 49700000 | −1.70210724 | 0.000192568 | C22orf34 |
| chr3_180650000_180700000 | chr3 | 180650000 | 180700000 | −1.981469572 | 0.00669927 | CCDC39-AS1 |
| chr12_4250000_4300000 | chr12 | 4250000 | 4300000 | −1.620717197 | 9.10271E−05 | CCND2; CCND2- |
| | | | | | | AS2; CCND2-AS1 |
| chr22_49450000_49500000 | chr22 | 49450000 | 49500000 | −1.318848091 | 0.000438484 | CTA-722E9.1 |
| chr7_137800000_137850000 | chr7 | 137800000 | 137850000 | −2.0076895 | 0.001975929 | DGKI |
| chr1_50400000_50450000 | chr1 | 50400000 | 50450000 | −2.173739606 | 0.008521108 | DMRTA2 |
| chr3_109300000_109350000 | chr3 | 109300000 | 109350000 | −1.756267344 | 0.000378183 | DPPA4 |
| chr2_176050000_176100000 | chr2 | 176050000 | 176100000 | −2.146183774 | 0.002719569 | EVX2 |
| chr8_138450000_138500000 | chr8 | 138450000 | 138500000 | −1.306132326 | 0.001657572 | FAM135B |
| chr10_15350000_15400000 | chr10 | 15350000 | 15400000 | −2.362442506 | 0.006687623 | FAM171A1 |
| chr12_4400000_4450000 | chr12 | 4400000 | 4450000 | −1.846372834 | 0.002990565 | FGF6 |
| chr13_38650000_38700000 | chr13 | 38650000 | 38700000 | −1.508090953 | 0.001731659 | FREM2 |
| chr3_180850000_180900000 | chr3 | 180850000 | 180900000 | −1.069123256 | 0.005602552 | FXR1; CCDC39 |
| chr13_93200000_93250000 | chr13 | 93200000 | 93250000 | −2.285846356 | 0.003718984 | GPC6; RP11- |
| | | | | | | 632L2.2 |
| chr1_185650000_185700000 | chr1 | 185650000 | 185700000 | −1.647231215 | 0.000103558 | GS1-204I12.4 |
| chr1_185700000_185750000 | chr1 | 185700000 | 185750000 | −1.785847906 | 0.006723134 | HMCN1 |
| chr12_65800000_65850000 | chr12 | 65800000 | 65850000 | −1.682201634 | 5.28266E−06 | HMGA2 |
| chr13_46850000_46900000 | chr13 | 46850000 | 46900000 | −2.346290612 | 0.000192466 | HTR2A- |
| | | | | | | AS1; HTR2A |
| chr7_155050000_155100000 | chr7 | 155050000 | 155100000 | −1.033354796 | 0.005016782 | HTR5A; |
| | | | | | | HTR5A-AS1 |
| chr6_19800000_19850000 | chr6 | 19800000 | 19850000 | −2.886781752 | 1.40279E−05 | ID4; RP4- |
| | | | | | | 625H18.2 |
| chr7_41650000_41700000 | chr7 | 41650000 | 41700000 | −1.842284909 | 0.009359169 | INHBA-AS1 |
| chr16_87600000_87650000 | chr16 | 87600000 | 87650000 | 6.044427807 | 0.001484001 | JPH3; RP11- |
| | | | | | | 482M8.3 |
| chr1_245150000_245200000 | chr1 | 245150000 | 245200000 | −1.447137008 | 0.001187658 | KIF26B |
| chr20_9500000_9550000 | chr20 | 9500000 | 9550000 | −2.240086336 | 0.001081259 | LAMP5; |
| | | | | | | LAMP5-AS1 |
| chr12_71400000_71450000 | chr12 | 71400000 | 71450000 | −1.503134448 | 0.001064358 | LGR5; TSPAN8 |
| chr12_113450000_113500000 | chr12 | 113450000 | 113500000 | −2.132082986 | 5.69317E−06 | LHX5- |
| | | | | | | AS1; LHX5 |
| chr20_22550000_22600000 | chr20 | 22550000 | 22600000 | −1.546379953 | 0.000162491 | LINC00261; |
| | | | | | | FOXA2 |
| chr13_111900000_111950000 | chr13 | 111900000 | 111950000 | −1.42640496 | 8.01128E−07 | LINC00354 |
| chr13_111850000_111900000 | chr13 | 111850000 | 111900000 | −1.454066964 | 1.5421E−05 | LINC00354 |
| chr13_94700000_94750000 | chr13 | 94700000 | 94750000 | −1.763016564 | 0.000192466 | LINC00391; |
| | | | | | | SOX21; |
| | | | | | | SOX21-AS1 |
| chr13_110050000_110100000 | chr13 | 110050000 | 110100000 | −1.221110862 | 0.007728727 | LINC00396; |
| | | | | | | RP11-403A3.3 |
| chr13_112100000_112150000 | chr13 | 112100000 | 112150000 | −1.621889747 | 4.63058E−08 | LINC00404; |
| | | | | | | LINC00403 |
| chr13_27950000_28000000 | chr13 | 27950000 | 28000000 | −2.073531841 | 6.88489E−05 | LINC00543; |
| | | | | | | CDX2; URAD |
| chr13_106600000_106650000 | chr13 | 106600000 | 106650000 | −1.608269125 | 0.000142296 | LINC00551 |
| chr13_94950000_95000000 | chr13 | 94950000 | 95000000 | −1.023669002 | 0.003579694 | LINC00557 |
| chr20_11850000_11900000 | chr20 | 11850000 | 11900000 | −1.105660533 | 0.009830827 | LINC00687 |
| chr18_76500000_76550000 | chr18 | 76500000 | 76550000 | 3.14417894 | 0.006699728 | LINC00908 |
| chr12_125950000_126000000 | chr12 | 125950000 | 126000000 | 1.78976621 | 0.002425078 | LINC00939; |
| | | | | | | RP5-916L7.2 |
| chr6_27650000_27700000 | chr6 | 27650000 | 27700000 | −2.41213869 | 1.2738E−05 | LINC01012 |
| chr8_135200000_135250000 | chr8 | 135200000 | 135250000 | −1.356677165 | 0.001552571 | LINC01591 |
| chr8_55850000_55900000 | chr8 | 55850000 | 55900000 | −1.77751355 | 0.001200096 | LYN |
| chr13_35450000_35500000 | chr13 | 35450000 | 35500000 | −2.452530799 | 0.008053884 | MAB21L1 |
| chr15_37100000_37150000 | chr15 | 37100000 | 37150000 | −2.886160729 | 0.00207681 | MEIS2; |
| | | | | | | RP11-128A17.2 |
| chr6_152650000_152700000 | chr6 | 152650000 | 152700000 | −1.977804379 | 0.005558147 | MYCT1 |
| chr12_80700000_80750000 | chr12 | 80700000 | 80750000 | −1.987448666 | 0.007794749 | MYF6; MYF5 |
| chr13_109100000_109150000 | chr13 | 109100000 | 109150000 | −1.319253537 | 0.004458992 | MYO16-AS2 |
| chr3_173350000_173400000 | chr3 | 173350000 | 173400000 | −1.247525527 | 0.005817379 | NLGN1 |
| chr1_247400000_247450000 | chr1 | 247400000 | 247450000 | −1.978661469 | 1.70229E−05 | NLRP3 |
| chr2_100450000_100500000 | chr2 | 100450000 | 100500000 | −1.937628177 | 0.002467696 | NMS |
| chr7_8400000_8450000 | chr7 | 8400000 | 8450000 | −1.329936395 | 0.002988363 | NXPH1 |
| chr19_1750000_1800000 | chr19 | 1750000 | 1800000 | −1.322193809 | 0.004195209 | ONECUT3 |
| chr19_8700000_8750000 | chr19 | 8700000 | 8750000 | −2.241254815 | 0.00065375 | OR2Z1 |
| chr11_56450000_56500000 | chr11 | 56450000 | 56500000 | 2.618710783 | 0.006530622 | OR5M8 |
| chr3_152800000_152850000 | chr3 | 152800000 | 152850000 | −1.2221533 | 0.003930538 | P2RY1 |
| chr3_190100000_190150000 | chr3 | 190100000 | 190150000 | −2.003431672 | 8.67762E−11 | P3H2- |
| | | | | | | AS1; P3H2 |
| chr6_163150000_163200000 | chr6 | 163150000 | 163200000 | −2.132287682 | 0.000601613 | PACRG-AS3 |
| chr4_110600000_110650000 | chr4 | 110600000 | 110650000 | −2.233986224 | 4.41213E−08 | PANCR; PITX2 |
| chr13_52800000_52850000 | chr13 | 52800000 | 52850000 | −2.13165389 | 0.0078624 | PCDH8 |
| chr5_141350000141400000 | chr5 | 141350000 | 141400000 | −1.966780565 | 0.000441198 | PCDHGB1; |
| | | | | | | AC005618.8; |
| | | | | | | PCDHGA4; |
| | | | | | | PCDHGB2; |
| | | | | | | PCDHGA5; |
| | | | | | | PCDHGB3; |
| | | | | | | PCDHGA6; |
| | | | | | | PCDHGA7; |
| | | | | | | PCDHGB4; |
| | | | | | | PCDHGA8; |
| | | | | | | PCDHGB5 |
| chr20_17200000_17250000 | chr20 | 17200000 | 17250000 | −1.527371503 | 0.002020741 | PCSK2 |
| chr6_165950000_166000000 | chr6 | 165950000 | 166000000 | −3.599259591 | 0.000456302 | PDE10A; LINC00602; |
| | | | | | | LINC00473 |
| chr13_27900000_27950000 | chr13 | 27900000 | 27950000 | −1.312582716 | 0.000327992 | PDX1; PDX1- |
| | | | | | | AS1; ATP5EP2 |
| chr1_230400000_230450000 | chr1 | 230400000 | 230450000 | −1.410878458 | 0.003961219 | PGBD5; RP4- |
| | | | | | | 553F17.1 |
| chr1_242500000_242550000 | chr1 | 242500000 | 242550000 | −1.51727972 | 0.001192968 | PLD5 |
| chr6_27300000_27350000 | chr6 | 27300000 | 27350000 | −1.678958925 | 0.001942382 | POM121L2 |
| chr2_104850000_104900000 | chr2 | 104850000 | 104900000 | −2.066667427 | 5.24907E−12 | POU3F3; |
| | | | | | | AC018730.4 |
| chr7_114050000_114100000 | chr7 | 114050000 | 114100000 | −2.542475055 | 0.003641055 | PPP1R3A; FOXP2 |
| chr6_27200000_27250000 | chr6 | 27200000 | 27250000 | −2.507563143 | 1.63629E−06 | PRSS16 |
| chr20_43150000_43200000 | chr20 | 43150000 | 43200000 | −1.300382602 | 0.000711067 | PTPRT; RP1- |
| | | | | | | 269M15.3 |
| chr3_157400000_157450000 | chr3 | 157400000 | 157450000 | −1.610166224 | 0.000646074 | PTX3 |
| chr12_85800000_85850000 | chr12 | 85800000 | 85850000 | −1.556813897 | 0.005602552 | RASSF9 |
| chr12_106550000_106600000 | chr12 | 106550000 | 106600000 | −3.482875288 | 0.000401027 | RFX4 |
| chr6_27400000_27450000 | chr6 | 27400000 | 27450000 | −2.858487228 | 0.006662885 | RP1-153G14.4 |
| chr22_49550000_49600000 | chr22 | 49550000 | 49600000 | −1.461211419 | 0.00242786 | RP1-29C18.9 |
| chr19_6950000_7000000 | chr19 | 6950000 | 7000000 | −1.622748254 | 0.009091308 | RP11-1137G4.3 |
| chr13_97650000_97700000 | chr13 | 97650000 | 97700000 | −1.890672358 | 0.000871682 | RP11-120E13.1 |
| chr15_37050000_37100000 | chr15 | 37050000 | 37100000 | −1.474527595 | 0.006723134 | RP11-128A17.1 |
| chr13_108300000_108350000 | chr13 | 108300000 | 108350000 | −1.83559742 | 9.45005E−06 | RP11-153124.5 |
| chr20_53150000_53200000 | chr20 | 53150000 | 53200000 | −1.565202142 | 0.000312465 | RP11-15M15.1 |
| chr20_53200000_53250000 | chr20 | 53200000 | 53250000 | −2.008203642 | 0.001578241 | RP11-15M15.2 |
| chr16_1050000_1100000 | chr16 | 1050000 | 1100000 | −3.796362993 | 0.000205873 | RP11-161M6.5; |
| | | | | | | SSTR5 |
| chr15_98300000_98350000 | chr15 | 98300000 | 98350000 | −1.164458317 | 0.008716839 | RP11-167B3.2; |
| | | | | | | RP11-167B3.3 |
| chr18_76450000_76500000 | chr18 | 76450000 | 76500000 | 3.808268454 | 0.004302055 | RP11-17M16.2; |
| | | | | | | ZNF516; |
| | | | | | | C18orf65 |
| chr3_27700000_27750000 | chr3 | 27700000 | 27750000 | −2.275270757 | 0.002932752 | RP11-222K16.2; |
| | | | | | | EOMES |
| chr6_165750000_165800000 | chr6 | 165750000 | 165800000 | −3.060971129 | 0.000529767 | RP11-252P19.1 |
| chr13_106900000_106950000 | chr13 | 106900000 | 106950000 | −1.455415959 | 4.32309E−05 | RP11-282A11.4 |
| chr16_9650000_9700000 | chr16 | 9650000 | 9700000 | −1.308245401 | 0.003337905 | RP11-297M9.1 |
| chr9_27350000_27400000 | chr9 | 27350000 | 27400000 | −2.66133415 | 0.008110144 | RP11-298E2.2 |
| chr15_93250000_93300000 | chr15 | 93250000 | 93300000 | −1.951659662 | 0.000214397 | RP11-326A13.1 |
| chr14_62050000_62100000 | chr14 | 62050000 | 62100000 | −2.063527295 | 0.000463386 | RP11-355I22.5 |
| chr15_98600000_98650000 | chr15 | 98600000 | 98650000 | −1.088787553 | 0.007608875 | RP11-35O15.1; IGF1R |
| chr12_66000000_66050000 | chr12 | 66000000 | 66050000 | −1.648841863 | 0.000871682 | RP11-366L20.4 |
| chr8_139450000_139500000 | chr8 | 139450000 | 139500000 | −1.415952706 | 0.000237757 | RP11-398H6.1 |
| chr20_61750000_61800000 | chr20 | 61750000 | 61800000 | −2.31659018 | 5.42369E−06 | RP11-429E11.2 |
| chr5_2800000_2850000 | chr5 | 2800000 | 2850000 | 1.528394863 | 0.001844069 | RP11-468D11.1 |
| chr3_181700000_181750000 | chr3 | 181700000 | 181750000 | −2.121935643 | 1.90521E−06 | RP11-4B14.3; SOX2 |
| chr18_76350000_76400000 | chr18 | 76350000 | 76400000 | 4.515713494 | 0.006771896 | RP11-504I13.3 |
| chr16_86250000_86300000 | chr16 | 86250000 | 86300000 | −1.288186264 | 0.002220408 | RP11-514D23.2; |
| | | | | | | RP11-514D23.1 |
| chr6_750000_800000 | chr6 | 750000 | 800000 | −1.489860156 | 0.003357151 | RP11-532F6.5; |
| | | | | | | RP11-284J1.1 |
| chr4_121050000_121100000 | chr4 | 121050000 | 121100000 | −2.46884053 | 0.005602552 | RP11-647P12.1; |
| | | | | | | NDNF |
| chr3_14735000_0147400000 | chr3 | 147350000 | 147400000 | −1.807261743 | 1.54435E−07 | RP11-649A16.1; |
| | | | | | | ZIC4-AS1; ZIC1 |
| chr13_111550000_111600000 | chr13 | 111550000 | 111600000 | −1.434510305 | 1.99936E−06 | RP11-65D24.2; |
| | | | | | | RP11-65D24.1 |
| chr12_3850000_3900000 | chr12 | 3850000 | 3900000 | −1.851525526 | 0.008716839 | RP11-664D1.1; |
| | | | | | | PARP11 |
| chr3_143100000_143150000 | chr3 | 143100000 | 143150000 | −2.383437856 | 0.001775695 | RP11-80H8.3; |
| | | | | | | CHST2 |
| chr6_1350000_1400000 | chr6 | 1350000 | 1400000 | −1.835048867 | 0.006649038 | RP4-668J24.2; |
| | | | | | | FOXF2 |
| chr20_53450000_53500000 | chr20 | 53450000 | 53500000 | −1.805177037 | 0.001077977 | RP4-678D15.1 |
| chr6_1000000_1050000 | chr6 | 1000000 | 1050000 | −2.253873861 | 0.000611119 | RP5-856G1.1 |
| chr22_49700000_49750000 | chr22 | 49700000 | 49750000 | −1.412496607 | 4.96159E−05 | RP5-983L19.2 |
| chr6_147500000_147550000 | chr6 | 147500000 | 147550000 | −1.712312073 | 0.001495858 | SAMD5 |
| chr2_199450000_199500000 | chr2 | 199450000 | 199500000 | −1.207157505 | 0.000463386 | SATB2-AS1; |
| | | | | | | SATB2 |
| chr13_36650000_36700000 | chr13 | 36650000 | 36700000 | −2.452764296 | 0.000497791 | SERTM1 |
| chr14_36500000_36550000 | chr14 | 36500000 | 36550000 | −1.787380228 | 0.000231991 | SFTA3; |
| | | | | | | NKX2-1-AS1; |
| | | | | | | NKX2-1 |
| chr8_116900000_116950000 | chr8 | 116900000 | 116950000 | 1.135842164 | 0.006723134 | SLC30A8 |
| chr20_38700000_38750000 | chr20 | 38700000 | 38750000 | −1.661882951 | 2.0545E−05 | SLC32A1 |
| chr17_35200000_35250000 | chr17 | 35200000 | 35250000 | −2.570172587 | 0.004556519 | SLFN5 |
| chr20_16700000_16750000 | chr20 | 16700000 | 16750000 | −1.773343704 | 0.000573451 | SNRPB2 |
| chr12_93550000_93600000 | chr12 | 93550000 | 93600000 | −2.273742552 | 0.001193885 | SOCS2 |
| chr13_112050000_112100000 | chr13 | 112050000 | 112100000 | −1.54035255 | 2.9727E−06 | SOX1; RP11- |
| | | | | | | 450H6.3 |
| chr3_137750000_137800000 | chr3 | 137750000 | 137800000 | −1.278075694 | 0.003399471 | SOX14; |
| | | | | | | LINC01210 |
| chr7_20750000_20800000 | chr7 | 20750000 | 20800000 | −1.527295553 | 0.001266845 | SP8 |
| chr3_98700000_98750000 | chr3 | 98700000 | 98750000 | −1.826798701 | 0.000194807 | ST3GAL6; |
| | | | | | | ST3GAL6-AS1 |
| chr5_76050000_76100000 | chr5 | 76050000 | 76100000 | −2.174795073 | 0.006026183 | SV2C; |
| | | | | | | CTC-235G5.3 |
| chr12_114400000_114450000 | chr12 | 114400000 | 114450000 | −2.03528457 | 0.000289181 | TBX5-AS1; |
| | | | | | | TBX5 |
| chr6_50700000_50750000 | chr6 | 50700000 | 50750000 | −1.851636154 | 0.009830827 | TFAP2D |
| chr7_1200000_1250000 | chr7 | 1200000 | 1250000 | −1.99024942 | 0.000754707 | UNCX |
| chr20_61350000_61400000 | chr20 | 61350000 | 61400000 | −2.121158524 | 0.003399471 | WI2-8325B5.1 |
| chr2_206250000_206300000 | chr2 | 206250000 | 206300000 | −1.008047191 | 0.002168921 | ZDBF2 |
| chr3_147400000_147450000 | chr3 | 147400000 | 147450000 | −1.990454986 | 6.03894E−08 | ZIC4 |
| chr2_179850000_179900000 | chr2 | 179850000 | 179900000 | −2.312390261 | 0.000817083 | ZNF385B |
| chr19_30200000_30250000 | chr19 | 30200000 | 30250000 | −1.459259752 | 0.000517633 | ZNF536; |
| | | | | | | AC005597.1 |

TABLE 5
genomic regions depleted in H3K27me3 enrichment in resistant or resistant-like
cells compared to sensitive cells for Tamoxifen treatment non-overlapping a transcription
start site, q-value < 0.01 (sixth column) and |log2FC| > 1 (fifth
column). The first column relates to the identification numbers of the genomic sequences
using the reference genome GRCh38 (GeneBank assembly accession GCA_000001405.15),
second column shows the chromosome number. The third and fourth columns relate
to the start and end position of genomic sequence respectively.

| | | | | log2FC. res. | adj. |
|---|---|---|---|---|---|
| ID | chr | Start | end | vs. sens | pval |
| chr1_50300000_50350000 | chr1 | 50300000 | 50350000 | −2.27444 | 0.000646 |
| chr1_233800000_233850000 | chr1 | 2.34E+08 | 2.34E+08 | −1.53646 | 0.005817 |
| chr1_3000000_3050000 | chr1 | 3000000 | 3050000 | −4.26395 | 0.009885 |
| chr1_208250000_208300000 | chr1 | 2.08E+08 | 2.08E+08 | −2.06865 | 0.000887 |
| chr1_218200000_218250000 | chr1 | 2.18E+08 | 2.18E+08 | −1.3512 | 0.004311 |
| chr1_50100000_50150000 | chr1 | 50100000 | 50150000 | −1.35231 | 0.003382 |
| chr1_205600000_205650000 | chr1 | 2.06E+08 | 2.06E+08 | 7.705603 | 0.002468 |
| chr1_214050000_214100000 | chr1 | 2.14E+08 | 2.14E+08 | −1.6949 | 0.009913 |
| chr1_216800000_216850000 | chr1 | 2.17E+08 | 2.17E+08 | −1.74819 | 0.006336 |
| chr1_218150000_218200000 | chr1 | 2.18E+08 | 2.18E+08 | −2.27144 | 0.000176 |
| chr1_236000000_236050000 | chr1 | 2.36E+08 | 2.36E+08 | 4.596965 | 0.004442 |
| chr10_6250000_6300000 | chr10 | 6250000 | 6300000 | Inf | 0.000817 |
| chr10_52650000_52700000 | chr10 | 52650000 | 52700000 | −2.04763 | 0.004195 |
| chr10_98800000_98850000 | chr10 | 98800000 | 98850000 | −1.88461 | 0.001193 |
| chr10_120800000_120850000 | chr10 | 1.21E+08 | 1.21E+08 | −1.27165 | 0.002475 |
| chr10_129850000_129900000 | chr10 | 1.3E+08 | 1.3E+08 | −2.10619 | 0.000386 |
| chr10_1650000_1700000 | chr10 | 1650000 | 1700000 | −1.826 | 0.004858 |
| chr10_11200000_11250000 | chr10 | 11200000 | 11250000 | −1.23336 | 0.006598 |
| chr10_16450000_16500000 | chr10 | 16450000 | 16500000 | 4.711479 | 0.000518 |
| chr10_16950000_17000000 | chr10 | 16950000 | 17000000 | −1.9115 | 0.006595 |
| chr10_100600000_100650000 | chr10 | 1.01E+08 | 1.01E+08 | −1.53962 | 0.009725 |
| chr10_104650000_104700000 | chr10 | 1.05E+08 | 1.05E+08 | −1.59625 | 0.006598 |
| chr10_104900000_104950000 | chr10 | 1.05E+08 | 1.05E+08 | −2.50476 | 0.003817 |
| chr10_120200000_120250000 | chr10 | 1.2E+08 | 1.2E+08 | −2.20865 | 0.006702 |
| chr10_120250000_120300000 | chr10 | 1.2E+08 | 1.2E+08 | −2.47396 | 5.3E−05 |
| chr10_128250000_128300000 | chr10 | 1.28E+08 | 1.28E+08 | −1.23199 | 0.008956 |
| chr10_129000000_129050000 | chr10 | 1.29E+08 | 1.29E+08 | −1.46375 | 0.002601 |
| chr10_129900000_129950000 | chr10 | 1.3E+08 | 1.3E+08 | −2.6199 | 0.009582 |
| chr10_131400000_131450000 | chr10 | 1.31E+08 | 1.31E+08 | −2.02373 | 0.008717 |
| chr10_131500000_131550000 | chr10 | 1.32E+08 | 1.32E+08 | −1.74519 | 0.002425 |
| chr11_41150000_41200000 | chr11 | 41150000 | 41200000 | 4.308754 | 0.00222 |
| chr11_44150000_44200000 | chr11 | 44150000 | 44200000 | 2.263477 | 0.002425 |
| chr11_73000000_73050000 | chr11 | 73000000 | 73050000 | 2.052239 | 0.00851 |
| chr11_19550000_19600000 | chr11 | 19550000 | 19600000 | 1.639311 | 0.008676 |
| chr11_19800000_19850000 | chr11 | 19800000 | 19850000 | 3.667903 | 0.001817 |
| chr11_33700000_33750000 | chr11 | 33700000 | 33750000 | 6.906491 | 0.002477 |
| chr11_55900000_55950000 | chr11 | 55900000 | 55950000 | 2.22882 | 0.009582 |
| chr11_96100000_96150000 | chr11 | 96100000 | 96150000 | Inf | 0.006531 |
| chr11_97150000_97200000 | chr11 | 97150000 | 97200000 | 4.817789 | 0.006723 |
| chr12_58700000_58750000 | chr12 | 58700000 | 58750000 | −2.47645 | 0.006598 |
| chr12_3900000_3950000 | chr12 | 3900000 | 3950000 | −1.82507 | 0.000413 |
| chr12_5600000_5650000 | chr12 | 5600000 | 5650000 | −2.0929 | 0.003961 |
| chr12_5850000_5900000 | chr12 | 5850000 | 5900000 | −1.67153 | 0.009885 |
| chr12_29600000_29650000 | chr12 | 29600000 | 29650000 | 3.714737 | 0.006723 |
| chr12_34300000_34350000 | chr12 | 34300000 | 34350000 | −2.34337 | 1.56E−06 |
| chr12_46750000_46800000 | chr12 | 46750000 | 46800000 | −2.31156 | 0.00457 |
| chr12_65750000_65800000 | chr12 | 65750000 | 65800000 | −1.92016 | 0.002326 |
| chr12_100750000_100800000 | chr12 | 1.01E+08 | 1.01E+08 | −2.6117 | 2.5E−05 |
| chr12_132350000_132400000 | chr12 | 1.32E+08 | 1.32E+08 | −3.11633 | 1.59E−06 |
| chr12_3950000_4000000 | chr12 | 3950000 | 4000000 | −2.09612 | 4.32E−05 |
| chr12_4000000_4050000 | chr12 | 4000000 | 4050000 | −2.19991 | 0.001643 |
| chr12_4100000_4150000 | chr12 | 4100000 | 4150000 | −2.02533 | 3.63E−05 |
| chr12_4200000_4250000 | chr12 | 4200000 | 4250000 | −1.50604 | 0.0013 |
| chr12_5800000_5850000 | chr12 | 5800000 | 5850000 | −1.22421 | 0.005161 |
| chr12_34350000_34400000 | chr12 | 34350000 | 34400000 | −3.22278 | 6.39E−08 |
| chr12_65700000_65750000 | chr12 | 65700000 | 65750000 | −1.60738 | 0.000351 |
| chr12_72000000_72050000 | chr12 | 72000000 | 72050000 | −1.03208 | 0.003382 |
| chr12_94150000_94200000 | chr12 | 94150000 | 94200000 | −2.15491 | 0.002757 |
| chr12_114350000_114400000 | chr12 | 1.14E+08 | 1.14E+08 | −2.51296 | 0.001299 |
| chr12_128900000_128950000 | chr12 | 1.29E+08 | 1.29E+08 | −1.75601 | 0.006889 |
| chr13_110100000_110150000 | chr13 | 1.1E+08 | 1.1E+08 | −1.03037 | 0.00454 |
| chr13_112150000_112200000 | chr13 | 1.12E+08 | 1.12E+08 | −1.22965 | 0.004459 |
| chr13_95000000_95050000 | chr13 | 95000000 | 95050000 | −1.37807 | 0.001645 |
| chr13_95250000_95300000 | chr13 | 95250000 | 95300000 | −1.46994 | 0.004485 |
| chr13_105600000_105650000 | chr13 | 1.06E+08 | 1.06E+08 | −1.18454 | 0.006723 |
| chr13_106400000_106450000 | chr13 | 1.06E+08 | 1.06E+08 | −2.05397 | 5.25E−06 |
| chr13_107050000_107100000 | chr13 | 1.07E+08 | 1.07E+08 | −1.49509 | 0.006598 |
| chr13_110200000_110250000 | chr13 | 1.1E+08 | 1.1E+08 | −1.25853 | 0.004502 |
| chr13_111400000_111450000 | chr13 | 1.11E+08 | 1.11E+08 | −2.23179 | 2.49E−11 |
| chr13_111500000_111550000 | chr13 | 1.12E+08 | 1.12E+08 | −2.33653 | 2.49E−11 |
| chr13_21700000_21750000 | chr13 | 21700000 | 21750000 | −1.36597 | 0.007795 |
| chr13_81850000_81900000 | chr13 | 81850000 | 81900000 | −1.4008 | 0.008956 |
| chr13_93900000_93950000 | chr13 | 93900000 | 93950000 | −2.71279 | 0.001961 |
| chr13_94650000_94700000 | chr13 | 94650000 | 94700000 | −1.6031 | 0.009091 |
| chr13_95200000_95250000 | chr13 | 95200000 | 95250000 | −2.62405 | 1.02E−06 |
| chr13_95350000_95400000 | chr13 | 95350000 | 95400000 | −2.02451 | 0.004998 |
| chr13_97700000_97750000 | chr13 | 97700000 | 97750000 | −2.03766 | 0.008313 |
| chr13_106450000_106500000 | chr13 | 1.06E+08 | 1.07E+08 | −2.24575 | 5.82E−06 |
| chr13_106700000_106750000 | chr13 | 1.07E+08 | 1.07E+08 | −1.58775 | 0.001819 |
| chr13_106850000_106900000 | chr13 | 1.07E+08 | 1.07E+08 | −1.88011 | 6.02E−07 |
| chr13_107250000_107300000 | chr13 | 1.07E+08 | 1.07E+08 | 2.046501 | 0.003399 |
| chr13_107400000_107450000 | chr13 | 1.07E+08 | 1.07E+08 | 2.640243 | 0.006598 |
| chr13_107550000_107600000 | chr13 | 1.08E+08 | 1.08E+08 | 2.441049 | 0.001193 |
| chr13_110150000_110200000 | chr13 | 1.1E+08 | 1.1E+08 | −1.46942 | 6.97E−07 |
| chr13_110250000_110300000 | chr13 | 1.1E+08 | 1.1E+08 | −1.21193 | 5.19E−06 |
| chr13_111450000_111500000 | chr13 | 1.11E+08 | 1.12E+08 | −1.76656 | 8.2E−06 |
| chr13_111600000_111650000 | chr13 | 1.12E+08 | 1.12E+08 | −1.33043 | 5.42E−06 |
| chr13_111950000_112000000 | chr13 | 1.12E+08 | 1.12E+08 | −1.25556 | 0.000518 |
| chr13_112000000_112050000 | chr13 | 1.12E+08 | 1.12E+08 | −1.52171 | 6.77E−06 |
| chr14_85100000_85150000 | chr14 | 85100000 | 85150000 | 4.05792 | 0.004518 |
| chr15_93450000_93500000 | chr15 | 93450000 | 93500000 | −2.19157 | 0.007707 |
| chr15_33500000_33550000 | chr15 | 33500000 | 33550000 | −2.75694 | 0.000239 |
| chr15_36850000_36900000 | chr15 | 36850000 | 36900000 | −2.34828 | 0.008339 |
| chr15_59950000_60000000 | chr15 | 59950000 | 60000000 | −1.93742 | 0.000162 |
| chr15_62950000_63000000 | chr15 | 62950000 | 63000000 | 2.084963 | 0.006597 |
| chr15_67550000_67600000 | chr15 | 67550000 | 67600000 | 5.545063 | 0.006723 |
| chr15_96350000_96400000 | chr15 | 96350000 | 96400000 | −2.55404 | 0.006723 |
| chr16_1000000_1050000 | chr16 | 1000000 | 1050000 | −1.96243 | 0.003399 |
| chr16_5100000_5150000 | chr16 | 5100000 | 5150000 | −2.58033 | 0.003357 |
| chr16_5350000_5400000 | chr16 | 5350000 | 5400000 | −1.31318 | 0.005519 |
| chr16_10200000_10250000 | chr16 | 10200000 | 10250000 | −1.56133 | 0.006723 |
| chr16_30000000_30050000 | chr16 | 30000000 | 30050000 | 2.721727 | 0.006598 |
| chr16_5150000_5200000 | chr16 | 5150000 | 5200000 | −2.11623 | 0.006789 |
| chr16_17850000_17900000 | chr16 | 17850000 | 17900000 | −1.73225 | 0.009263 |
| chr16_24950000_25000000 | chr16 | 24950000 | 25000000 | 6.035399 | 0.002316 |
| chr16_63500000_63550000 | chr16 | 63500000 | 63550000 | 2.548683 | 0.007485 |
| chr16_67950000_68000000 | chr16 | 67950000 | 68000000 | 3.409111 | 0.006723 |
| chr16_86400000_86450000 | chr16 | 86400000 | 86450000 | −1.3623 | 0.002229 |
| chr16_86450000_86500000 | chr16 | 86450000 | 86500000 | −1.00518 | 0.009455 |
| chr17_55550000_55600000 | chr17 | 55550000 | 55600000 | −2.03352 | 0.00263 |
| chr17_78600000_78650000 | chr17 | 78600000 | 78650000 | Inf | 0.009913 |
| chr17_12800000_12850000 | chr17 | 12800000 | 12850000 | 4.190049 | 0.006723 |
| chr17_35250000_35300000 | chr17 | 35250000 | 35300000 | −1.40937 | 0.005483 |
| chr17_48350000_48400000 | chr17 | 48350000 | 48400000 | 1.611228 | 0.008956 |
| chr17_12900000_12950000 | chr17 | 12900000 | 12950000 | 7.406539 | 0.006608 |
| chr17_15400000_15450000 | chr17 | 15400000 | 15450000 | Inf | 0.009913 |
| chr17_49100000_49150000 | chr17 | 49100000 | 49150000 | 3.082998 | 0.00457 |
| chr17_49200000_49250000 | chr17 | 49200000 | 49250000 | 1.741867 | 0.006129 |
| chr17_58100000_58150000 | chr17 | 58100000 | 58150000 | −3.15055 | 0.001064 |
| chr17_64400000_64450000 | chr17 | 64400000 | 64450000 | 2.861662 | 0.008251 |
| chr17_65050000_65100000 | chr17 | 65050000 | 65100000 | 4.034173 | 0.004442 |
| chr18_7300000_7350000 | chr18 | 7300000 | 7350000 | 3.794262 | 0.001299 |
| chr18_72350000_72400000 | chr18 | 72350000 | 72400000 | 2.444372 | 0.007795 |
| chr18_8250000_8300000 | chr18 | 8250000 | 8300000 | 3.384444 | 0.001142 |
| chr18_55950000_56000000 | chr18 | 55950000 | 56000000 | 1.7584 | 0.008717 |
| chr18_66300000_66350000 | chr18 | 66300000 | 66350000 | Inf | 0.003931 |
| chr18_76400000_76450000 | chr18 | 76400000 | 76450000 | Inf | 6.47E−06 |
| chr18_1650000_1700000 | chr18 | 1650000 | 1700000 | Inf | 0.00237 |
| chr18_7250000_7300000 | chr18 | 7250000 | 7300000 | 2.971319 | 0.009725 |
| chr18_7650000_7700000 | chr18 | 7650000 | 7700000 | 4.957834 | 0.004326 |
| chr18_7750000_7800000 | chr18 | 7750000 | 7800000 | 3.838077 | 5.05E−05 |
| chr18_8300000_8350000 | chr18 | 8300000 | 8350000 | 3.852626 | 0.001204 |
| chr18_8450000_8500000 | chr18 | 8450000 | 8500000 | 5.790625 | 0.002507 |
| chr18_55850000_55900000 | chr18 | 55850000 | 55900000 | 2.920274 | 0.006723 |
| chr18_70850000_70900000 | chr18 | 70850000 | 70900000 | 3.178989 | 0.00418 |
| chr18_74750000_74800000 | chr18 | 74750000 | 74800000 | 5.426865 | 0.001498 |
| chr18_76550000_76600000 | chr18 | 76550000 | 76600000 | 3.778667 | 0.003045 |
| chr18_78850000_78900000 | chr18 | 78850000 | 78900000 | −1.58199 | 0.008172 |
| chr19_30400000_30450000 | chr19 | 30400000 | 30450000 | −1.58087 | 0.005218 |
| chr19_6550000_6600000 | chr19 | 6550000 | 6600000 | −2.40562 | 0.000753 |
| chr19_15550000_15600000 | chr19 | 15550000 | 15600000 | 4.533398 | 0.004149 |
| chr19_30250000_30300000 | chr19 | 30250000 | 30300000 | −1.45465 | 0.006598 |
| chr19_53700000_53750000 | chr19 | 53700000 | 53750000 | −1.78522 | 0.005808 |
| chr19_13450000_13500000 | chr19 | 13450000 | 13500000 | −1.09164 | 0.001078 |
| chr19_30350000_30400000 | chr19 | 30350000 | 30400000 | −1.82743 | 0.001141 |
| chr19_30700000_30750000 | chr19 | 30700000 | 30750000 | −2.44868 | 0.007806 |
| chr19_36250000_36300000 | chr19 | 36250000 | 36300000 | −2.19478 | 0.002757 |
| chr2_103650000_103700000 | chr2 | 1.04E+08 | 1.04E+08 | 2.570426 | 0.001688 |
| chr2_191600000_191650000 | chr2 | 1.92E+08 | 1.92E+08 | 3.998397 | 0.004492 |
| chr2_208400000_208450000 | chr2 | 2.08E+08 | 2.08E+08 | −1.35774 | 0.001047 |
| chr2_221600000_221650000 | chr2 | 2.22E+08 | 2.22E+08 | 4.99261 | 0.004302 |
| chr2_2000000_2050000 | chr2 | 2000000 | 2050000 | −1.16157 | 0.007736 |
| chr2_55900000_55950000 | chr2 | 55900000 | 55950000 | 4.005817 | 0.007609 |
| chr2_94900000_94950000 | chr2 | 94900000 | 94950000 | −1.83643 | 0.000293 |
| chr2_104600000_104650000 | chr2 | 1.05E+08 | 1.05E+08 | −1.7376 | 0.000238 |
| chr2_104750000_104800000 | chr2 | 1.05E+08 | 1.05E+08 | −2.33966 | 9.21E−11 |
| chr2_104800000_104850000 | chr2 | 1.05E+08 | 1.05E+08 | −2.68583 | 6.98E−12 |
| chr2_104900000_104950000 | chr2 | 1.05E+08 | 1.05E+08 | −1.17285 | 0.000563 |
| chr2_105150000_105200000 | chr2 | 1.05E+08 | 1.05E+08 | −1.35262 | 1.76E−05 |
| chr2_117350000_117400000 | chr2 | 1.17E+08 | 1.17E+08 | 2.925259 | 0.009913 |
| chr2_167800000_167850000 | chr2 | 1.68E+08 | 1.68E+08 | −3.04535 | 0.00631 |
| chr2_178850000_178900000 | chr2 | 1.79E+08 | 1.79E+08 | −1.61882 | 0.000648 |
| chr2_191900000_191950000 | chr2 | 1.92E+08 | 1.92E+08 | −1.77335 | 0.003557 |
| chr2_196550000_196600000 | chr2 | 1.97E+08 | 1.97E+08 | 2.981685 | 0.003245 |
| chr2_212900000_212950000 | chr2 | 2.13E+08 | 2.13E+08 | 3.112512 | 0.003278 |
| chr2_222200000_222250000 | chr2 | 2.22E+08 | 2.22E+08 | 2.743459 | 0.009913 |
| chr20_16600000_16650000 | chr20 | 16600000 | 16650000 | −2.00757 | 8.01E−06 |
| chr20_17400000_17450000 | chr20 | 17400000 | 17450000 | −2.76226 | 0.003517 |
| chr20_22500000_22550000 | chr20 | 22500000 | 22550000 | −1.47685 | 0.004149 |
| chr20_30500000_30550000 | chr20 | 30500000 | 30550000 | −1.61886 | 2.08E−06 |
| chr20_41800000_41850000 | chr20 | 41800000 | 41850000 | −1.53181 | 0.008956 |
| chr20_43100000_43150000 | chr20 | 43100000 | 43150000 | −1.18022 | 0.009973 |
| chr20_46250000_46300000 | chr20 | 46250000 | 46300000 | −1.69097 | 1.45E−07 |
| chr20_47950000_48000000 | chr20 | 47950000 | 48000000 | 3.619931 | 0.003332 |
| chr20_53050000_53100000 | chr20 | 53050000 | 53100000 | −1.6348 | 0.001645 |
| chr20_53300000_53350000 | chr20 | 53300000 | 53350000 | −2.16323 | 0.000137 |
| chr20_54000000_54050000 | chr20 | 54000000 | 54050000 | −1.84639 | 0.000412 |
| chr20_54650000_54700000 | chr20 | 54650000 | 54700000 | −1.36144 | 0.002077 |
| chr20_61600000_61650000 | chr20 | 61600000 | 61650000 | −1.67627 | 8.19E−05 |
| chr20_5250000_5300000 | chr20 | 5250000 | 5300000 | −1.7435 | 0.002653 |
| chr20_16150000_16200000 | chr20 | 16150000 | 16200000 | −1.98098 | 0.000142 |
| chr20_16650000_16700000 | chr20 | 16650000 | 16700000 | −3.01782 | 1.27E−11 |
| chr20_29300000_29350000 | chr20 | 29300000 | 29350000 | −1.52718 | 0.007823 |
| chr20_31400000_31450000 | chr20 | 31400000 | 31450000 | −1.30858 | 0.001078 |
| chr20_48500000_48550000 | chr20 | 48500000 | 48550000 | −1.53027 | 0.006547 |
| chr20_53100000_53150000 | chr20 | 53100000 | 53150000 | −2.29674 | 5.05E−05 |
| chr20_53250000_53300000 | chr20 | 53250000 | 53300000 | −1.79113 | 1.94E−06 |
| chr20_53400000_53450000 | chr20 | 53400000 | 53450000 | −2.08729 | 7.15E−05 |
| chr20_53950000_54000000 | chr20 | 53950000 | 54000000 | −1.63164 | 0.002169 |
| chr20_54600000_54650000 | chr20 | 54600000 | 54650000 | −1.79364 | 5.42E−06 |
| chr20_61500000_61550000 | chr20 | 61500000 | 61550000 | −1.43189 | 0.000396 |
| chr20_61650000_61700000 | chr20 | 61650000 | 61700000 | −1.66781 | 0.000817 |
| chr20_61800000_61850000 | chr20 | 61800000 | 61850000 | −1.08792 | 0.001455 |
| chr21_8200000_8250000 | chr21 | 8200000 | 8250000 | −1.85166 | 0.000529 |
| chr22_49000000_49050000 | chr22 | 49000000 | 49050000 | −1.80251 | 0.009711 |
| chr22_49150000_49200000 | chr22 | 49150000 | 49200000 | −1.61112 | 0.000576 |
| chr22_11600000_11650000 | chr22 | 11600000 | 11650000 | −1.69893 | 0.006598 |
| chr22_37250000_37300000 | chr22 | 37250000 | 37300000 | 4.064293 | 0.003052 |
| chr22_49350000_49400000 | chr22 | 49350000 | 49400000 | −1.80874 | 1.74E−06 |
| chr22_49600000_49650000 | chr22 | 49600000 | 49650000 | −2.26098 | 0.003635 |
| chr3_93450000_93500000 | chr3 | 93450000 | 93500000 | 2.029094 | 1.84E−14 |
| chr3_114150000_114200000 | chr3 | 1.14E+08 | 1.14E+08 | −1.52853 | 0.006889 |
| chr3_170750000_170800000 | chr3 | 1.71E+08 | 1.71E+08 | −2.06231 | 8.25E−06 |
| chr3_177600000_177650000 | chr3 | 1.78E+08 | 1.78E+08 | 2.676783 | 0.009831 |
| chr3_190050000_190100000 | chr3 | 1.9E+08 | 1.9E+08 | −1.86847 | 1.27E−11 |
| chr3_190150000_190200000 | chr3 | 1.9E+08 | 1.9E+08 | −1.39949 | 0.005808 |
| chr3_124850000_124900000 | chr3 | 1.25E+08 | 1.25E+08 | 4.614469 | 0.006821 |
| chr3_135800000_135850000 | chr3 | 1.36E+08 | 1.36E+08 | −1.75079 | 0.000638 |
| chr3_159450000_159500000 | chr3 | 1.59E+08 | 1.6E+08 | −1.64507 | 0.000836 |
| chr3_170450000_170500000 | chr3 | 1.7E+08 | 1.71E+08 | −1.24688 | 0.008339 |
| chr3_180550000_180600000 | chr3 | 1.81E+08 | 1.81E+08 | −1.47515 | 0.006598 |
| chr3_189950000_190000000 | chr3 | 1.9E+08 | 1.9E+08 | −1.44713 | 0.002264 |
| chr3_190000000_190050000 | chr3 | 1.9E+08 | 1.9E+08 | −1.9527 | 1.58E−09 |
| chr4_77700000_77750000 | chr4 | 77700000 | 77750000 | 6.48854 | 0.001455 |
| chr4_80850000_80900000 | chr4 | 80850000 | 80900000 | 4.04117 | 0.002719 |
| chr4_94500000_94550000 | chr4 | 94500000 | 94550000 | 3.715718 | 0.007564 |
| chr4_116900000_116950000 | chr4 | 1.17E+08 | 1.17E+08 | 1.815073 | 0.005623 |
| chr4_152550000_152600000 | chr4 | 1.53E+08 | 1.53E+08 | Inf | 0.009913 |
| chr4_43550000_43600000 | chr4 | 43550000 | 43600000 | 2.128774 | 0.005524 |
| chr4_52600000_52650000 | chr4 | 52600000 | 52650000 | 4.458123 | 0.006723 |
| chr4_91800000_91850000 | chr4 | 91800000 | 91850000 | 4.844734 | 0.006723 |
| chr4_98450000_98500000 | chr4 | 98450000 | 98500000 | 4.886721 | 0.000775 |
| chr4_140300000_140350000 | chr4 | 1.4E+08 | 1.4E+08 | 4.30121 | 0.008717 |
| chr4_142550000_142600000 | chr4 | 1.43E+08 | 1.43E+08 | 2.914279 | 0.000574 |
| chr5_2750000_2800000 | chr5 | 2750000 | 2800000 | 3.203465 | 0.000896 |
| chr5_15950000_16000000 | chr5 | 15950000 | 16000000 | 2.991914 | 0.006598 |
| chr5_26700000_26750000 | chr5 | 26700000 | 26750000 | 4.140361 | 0.004442 |
| chr5_29600000_29650000 | chr5 | 29600000 | 29650000 | 2.497117 | 0.006723 |
| chr5_29850000_29900000 | chr5 | 29850000 | 29900000 | 3.446463 | 0.004582 |
| chr5_30900000_30950000 | chr5 | 30900000 | 30950000 | 2.107415 | 0.007795 |
| chr5_35900000_35950000 | chr5 | 35900000 | 35950000 | −2.69533 | 0.006598 |
| chr5_156100000_156150000 | chr5 | 1.56E+08 | 1.56E+08 | 3.417148 | 0.003988 |
| chr5_2700000_2750000 | chr5 | 2700000 | 2750000 | 2.878402 | 0.00753 |
| chr5_5250000_5300000 | chr5 | 5250000 | 5300000 | −1.61667 | 0.004557 |
| chr5_8950000_9000000 | chr5 | 8950000 | 9000000 | 3.028403 | 0.008054 |
| chr5_11150000_11200000 | chr5 | 11150000 | 11200000 | −1.49277 | 0.00272 |
| chr5_22950000_23000000 | chr5 | 22950000 | 23000000 | 1.382486 | 0.008082 |
| chr5_23650000_23700000 | chr5 | 23650000 | 23700000 | 3.424022 | 0.006101 |
| chr5_27600000_27650000 | chr5 | 27600000 | 27650000 | 3.385228 | 0.0025 |
| chr5_32000000_32050000 | chr5 | 32000000 | 32050000 | 4.414519 | 0.004278 |
| chr5_51850000_51900000 | chr5 | 51850000 | 51900000 | 2.174685 | 0.005626 |
| chr5_155400000_155450000 | chr5 | 1.55E+08 | 1.55E+08 | 6.080068 | 0.000794 |
| chr5_155500000_155550000 | chr5 | 1.56E+08 | 1.56E+08 | 3.488772 | 0.001514 |
| chr5_155600000_155650000 | chr5 | 1.56E+08 | 1.56E+08 | 1.637822 | 0.001193 |
| chr5_156150000_156200000 | chr5 | 1.56E+08 | 1.56E+08 | 2.469821 | 0.00523 |
| chr5_163000000_163050000 | chr5 | 1.63E+08 | 1.63E+08 | 2.126484 | 0.005808 |
| chr6_27250000_27300000 | chr6 | 27250000 | 27300000 | −2.35528 | 1.28E−10 |
| chr6_147800000_147850000 | chr6 | 1.48E+08 | 1.48E+08 | −2.36603 | 0.001365 |
| chr6_154100000_154150000 | chr6 | 1.54E+08 | 1.54E+08 | −2.42483 | 0.005817 |
| chr6_13200000_13250000 | chr6 | 13200000 | 13250000 | −2.04456 | 0.005105 |
| chr6_19650000_19700000 | chr6 | 19650000 | 19700000 | −2.30173 | 0.000558 |
| chr6_19750000_19800000 | chr6 | 19750000 | 19800000 | −2.11661 | 0.006547 |
| chr6_27500000_27550000 | chr6 | 27500000 | 27550000 | −1.5291 | 0.001188 |
| chr6_147550000_147600000 | chr6 | 1.48E+08 | 1.48E+08 | −1.83177 | 0.003399 |
| chr6_163300000_163350000 | chr6 | 1.63E+08 | 1.63E+08 | −1.81271 | 0.008956 |
| chr6_165800000_165850000 | chr6 | 1.66E+08 | 1.66E+08 | −2.10521 | 0.001869 |
| chr6_5700000_5750000 | chr6 | 5700000 | 5750000 | −2.91488 | 0.001086 |
| chr6_13150000_13200000 | chr6 | 13150000 | 13200000 | −2.2511 | 0.001103 |
| chr6_27550000_27600000 | chr6 | 27550000 | 27600000 | −2.02876 | 5.28E−06 |
| chr6_28450000_28500000 | chr6 | 28450000 | 28500000 | −1.39935 | 0.005756 |
| chr6_113300000_113350000 | chr6 | 1.13E+08 | 1.13E+08 | −2.29431 | 0.006723 |
| chr6_142800000_142850000 | chr6 | 1.43E+08 | 1.43E+08 | Inf | 0.009913 |
| chr6_149050000_149100000 | chr6 | 1.49E+08 | 1.49E+08 | 4.174323 | 0.00272 |
| chr6_149950000_150000000 | chr6 | 1.5E+08 | 1.5E+08 | −2.52815 | 0.006723 |
| chr6_163200000_163250000 | chr6 | 1.63E+08 | 1.63E+08 | −1.5007 | 0.006598 |
| chr6_165700000_165750000 | chr6 | 1.66E+08 | 1.66E+08 | −1.37764 | 0.00838 |
| chr6_165850000_165900000 | chr6 | 1.66E+08 | 1.66E+08 | −1.25978 | 0.003828 |
| chr7_350000_400000 | chr7 | 350000 | 400000 | −1.94243 | 0.007823 |
| chr7_25650000_25700000 | chr7 | 25650000 | 25700000 | −1.89403 | 0.002952 |
| chr7_28550000_28600000 | chr7 | 28550000 | 28600000 | −1.30155 | 0.000728 |
| chr7_28700000_28750000 | chr7 | 28700000 | 28750000 | 1.208141 | 0.004605 |
| chr7_67600000_67650000 | chr7 | 67600000 | 67650000 | −1.88612 | 4.42E−05 |
| chr7_71950000_72000000 | chr7 | 71950000 | 72000000 | −1.91158 | 0.006965 |
| chr7_85850000_85900000 | chr7 | 85850000 | 85900000 | 2.975909 | 0.006531 |
| chr7_137750000_137800000 | chr7 | 1.38E+08 | 1.38E+08 | −1.7686 | 0.001497 |
| chr7_154650000_154700000 | chr7 | 1.55E+08 | 1.55E+08 | −1.29207 | 0.008717 |
| chr7_154750000_154800000 | chr7 | 1.55E+08 | 1.55E+08 | −1.22899 | 0.0013 |
| chr7_157650000_157700000 | chr7 | 1.58E+08 | 1.58E+08 | −2.27173 | 1.94E−08 |
| chr7_157800000_157850000 | chr7 | 1.58E+08 | 1.58E+08 | −1.40633 | 0.000347 |
| chr7_157900000_157950000 | chr7 | 1.58E+08 | 1.58E+08 | −2.64511 | 6.39E−08 |
| chr7_158050000_158100000 | chr7 | 1.58E+08 | 1.58E+08 | −1.61231 | 0.006523 |
| chr7_9500000_9550000 | chr7 | 9500000 | 9550000 | 2.755232 | 0.003176 |
| chr7_22350000_22400000 | chr7 | 22350000 | 22400000 | 3.090301 | 0.003382 |
| chr7_28500000_28550000 | chr7 | 28500000 | 28550000 | −1.50048 | 0.000218 |
| chr7_28900000_28950000 | chr7 | 28900000 | 28950000 | 2.983892 | 0.008717 |
| chr7_46300000_46350000 | chr7 | 46300000 | 46350000 | −1.4642 | 0.006531 |
| chr7_54850000_54900000 | chr7 | 54850000 | 54900000 | −1.93326 | 0.003113 |
| chr7_54950000_55000000 | chr7 | 54950000 | 55000000 | −1.84169 | 3.2E−05 |
| chr7_68750000_68800000 | chr7 | 68750000 | 68800000 | −2.33353 | 0.001452 |
| chr7_69150000_69200000 | chr7 | 69150000 | 69200000 | −2.14782 | 0.009468 |
| chr7_71900000_71950000 | chr7 | 71900000 | 71950000 | −1.62366 | 0.006336 |
| chr7_72300000_72350000 | chr7 | 72300000 | 72350000 | −2.1927 | 2.67E−05 |
| chr7_137550000_137600000 | chr7 | 1.38E+08 | 1.38E+08 | −1.19825 | 0.004882 |
| chr7_141300000_141350000 | chr7 | 1.41E+08 | 1.41E+08 | 1.845589 | 0.009475 |
| chr7_154800000_154850000 | chr7 | 1.55E+08 | 1.55E+08 | −1.45302 | 0.000876 |
| chr7_155100000_155150000 | chr7 | 1.55E+08 | 1.55E+08 | −1.75702 | 0.000142 |
| chr7_156300000_156350000 | chr7 | 1.56E+08 | 1.56E+08 | −2.19384 | 0.005817 |
| chr7_157550000_157600000 | chr7 | 1.58E+08 | 1.58E+08 | −2.58479 | 1.02E−14 |
| chr7_157950000_158000000 | chr7 | 1.58E+08 | 1.58E+08 | −1.70186 | 4.99E−05 |
| chr7_158150000_158200000 | chr7 | 1.58E+08 | 1.58E+08 | −1.0201 | 0.006598 |
| chr8_69550000_69600000 | chr8 | 69550000 | 69600000 | 4.200584 | 0.009945 |
| chr8_101550000_101600000 | chr8 | 1.02E+08 | 1.02E+08 | 4.356731 | 0.002468 |
| chr8_108100000_108150000 | chr8 | 1.08E+08 | 1.08E+08 | −1.71217 | 0.009647 |
| chr8_113100000_113150000 | chr8 | 1.13E+08 | 1.13E+08 | 2.11603 | 0.000823 |
| chr8_113900000_113950000 | chr8 | 1.14E+08 | 1.14E+08 | 1.557284 | 0.000518 |
| chr8_115850000_115900000 | chr8 | 1.16E+08 | 1.16E+08 | 2.632803 | 8.78E−05 |
| chr8_117000000_117050000 | chr8 | 1.17E+08 | 1.17E+08 | 1.359241 | 0.008276 |
| chr8_117300000_117350000 | chr8 | 1.17E+08 | 1.17E+08 | 1.735591 | 0.001549 |
| chr8_117800000_117850000 | chr8 | 1.18E+08 | 1.18E+08 | 2.065241 | 0.005841 |
| chr8_119650000_119700000 | chr8 | 1.2E+08 | 1.2E+08 | 2.120311 | 0.004552 |
| chr8_120150000_120200000 | chr8 | 1.2E+08 | 1.2E+08 | 2.317158 | 0.008554 |
| chr8_135650000_135700000 | chr8 | 1.36E+08 | 1.36E+08 | −1.10769 | 0.009294 |
| chr8_139550000_139600000 | chr8 | 1.4E+08 | 1.4E+08 | −1.00952 | 0.006635 |
| chr8_17900000_17950000 | chr8 | 17900000 | 17950000 | Inf | 0.003931 |
| chr8_19600000_19650000 | chr8 | 19600000 | 19650000 | 2.556382 | 0.002229 |
| chr8_65600000_65650000 | chr8 | 65600000 | 65650000 | 5.881482 | 0.002497 |
| chr8_91400000_91450000 | chr8 | 91400000 | 91450000 | −1.05213 | 0.00523 |
| chr8_93600000_93650000 | chr8 | 93600000 | 93650000 | −1.7263 | 0.007237 |
| chr8_100450000_100500000 | chr8 | 1E+08 | 1.01E+08 | 3.690046 | 0.002933 |
| chr8_113550000_113600000 | chr8 | 1.14E+08 | 1.14E+08 | 2.342953 | 3.2E−05 |
| chr8_116150000_116200000 | chr8 | 1.16E+08 | 1.16E+08 | 1.928773 | 0.006723 |
| chr8_116450000_116500000 | chr8 | 1.16E+08 | 1.17E+08 | 2.859876 | 0.000325 |
| chr8_116800000_116850000 | chr8 | 1.17E+08 | 1.17E+08 | 2.268712 | 0.005266 |
| chr8_117500000_117550000 | chr8 | 1.18E+08 | 1.18E+08 | 1.303223 | 0.008554 |
| chr8_120750000_120800000 | chr8 | 1.21E+08 | 1.21E+08 | −1.56233 | 0.001455 |
| chr8_121800000_121850000 | chr8 | 1.22E+08 | 1.22E+08 | −1.25133 | 0.003045 |
| chr8_123650000_123700000 | chr8 | 1.24E+08 | 1.24E+08 | 2.161014 | 0.009457 |
| chr8_134250000_134300000 | chr8 | 1.34E+08 | 1.34E+08 | −1.37105 | 0.006598 |
| chr8_134400000_134450000 | chr8 | 1.34E+08 | 1.34E+08 | −1.19718 | 0.000845 |
| chr8_135700000_135750000 | chr8 | 1.36E+08 | 1.36E+08 | −1.28512 | 0.002425 |
| chr8_138700000_138750000 | chr8 | 1.39E+08 | 1.39E+08 | −2.07975 | 2.05E−05 |
| chr8_139000000_139050000 | chr8 | 1.39E+08 | 1.39E+08 | −1.22772 | 0.000353 |
| chr8_139200000_139250000 | chr8 | 1.39E+08 | 1.39E+08 | −1.16223 | 0.000872 |
| chr8_139250000_139300000 | chr8 | 1.39E+08 | 1.39E+08 | −1.68173 | 4.92E−06 |
| chr8_139400000_139450000 | chr8 | 1.39E+08 | 1.39E+08 | −1.26459 | 0.000624 |
| chr8_139500000_139550000 | chr8 | 1.4E+08 | 1.4E+08 | −1.58731 | 0.005695 |
| chr9_14800000_14850000 | chr9 | 14800000 | 14850000 | −1.83887 | 0.009831 |
| chr9_133600000_133650000 | chr9 | 1.34E+08 | 1.34E+08 | −2.69641 | 0.00321 |
| chrX_12950000_13000000 | chrX | 12950000 | 13000000 | 5.250651 | 0.006547 |
| chrX_34200000_34250000 | chrX | 34200000 | 34250000 | 2.418981 | 0.008189 |
| chrX_105250000_105300000 | chrX | 1.05E+08 | 1.05E+08 | Inf | 0.009913 |
| chrX_6500000_6550000 | chrX | 6500000 | 6550000 | 2.005637 | 0.003045 |
| chrX_8150000_8200000 | chrX | 8150000 | 8200000 | 3.205772 | 0.008096 |
| chrX_16650000_16700000 | chrX | 16650000 | 16700000 | Inf | 0.009913 |
| chrX_25850000_25900000 | chrX | 25850000 | 25900000 | 2.862063 | 0.009945 |

TABLE 6
genomic regions depleted in H3K27me3 enrichment in tamoxifen resistant or resistant-like cells
compared to sensitive cells overlapping a transcription start site of a gene known to promote
resistance to chemotherapy, q-value < 0.01 (sixth column) and |log2FC| > 1 (fifth
column). The first column relates to the identification numbers of the genomic sequences using the
reference genome GRCh38 (GeneBank assembly accession GCA_000001405.15), second column shows
the chromosome number. The third and fourth columns relate to the start and end position of
genomic sequence respectively. Last column indicates genes related to transcription start site.

| | | | | log2FC. res. | adj. | Associated |
|---|---|---|---|---|---|---|
| ID | chr | start | End | vs. sens | pval | gene |
| chr3_105350000_105400000 | chr3 | 1.05E+08 | 1.05E+08 | −1.64958 | 0.001942 | ALCAM |
| chr7_55000000_55050000 | chr7 | 55000000 | 55050000 | −2.59365 | 0.004558 | EGFR |
| chr7_45900000_45950000 | chr7 | 45900000 | 45950000 | −1.45397 | 0.004906 | IGFBP3 |
| chr4_54200000_54250000 | chr4 | 54200000 | 54250000 | −2.46464 | 0.006723 | PDGFRA |
| chr13_110300000_110350000 | chr13 | 1.1E+08 | 1.1E+08 | −1.50318 | 2.93E−05 | COL4A2; COL4A1 |
| chr7_23450000_23500000 | chr7 | 23450000 | 23500000 | −1.49815 | 3.2E−05 | IGF2BP3 |

1-3. (canceled)

4. A method for determining whether a patient suffering from a cancer is resistant or sensitive to treatment with a cancer drug, comprising:
detecting, in a tumor sample from the patient, a histone modification of at least one biomarker which comprises one or more genomic sequence(s) comprising the histone modification, wherein said one or more genomic sequence(s) is/are selected from the lists of Tables 1 to 6 and,
determining from the presence or absence of the histone modification of said at least one biomarker, whether the patient is resistant or sensitive to said treatment.

5. A method for determining whether a patient suffering from a cancer is resistant or sensitive to treatment with a cancer drug, comprising:
i) determining in a tumor sample from the patient, the expression level of a gene encoding a biomarker which comprises one or more genomic sequence(s) comprising a histone modification, wherein said histone modification is in a gene or in the proximity of said gene and said genomic sequence(s) is/are selected from the list of Tables 1 and 4,
ii) determining from the expression level of said gene whether the patient is resistant or sensitive to said treatment.

6. (canceled)

7. The method according to claim 4, wherein the cancer drug is a chemotherapy drug, and said one or more genomic sequence(s) are selected from the genomic sequences listed in Tables 1 to 3.

8. The method of claim 7 wherein said cancer drug is capecitabine.

9. The method according to claim 4, wherein said cancer drug is an anti-hormonal drug, and said one or more genomic sequence(s) are selected from the list of Tables 4 to 6.

10. The method of claim 9 wherein said anti-hormonal drug is tamoxifen.

11. The method according to claim 4, wherein said histone modification is associated with transcriptional activation.

12. The method of claim 11 wherein said histone modification is a loss of transcriptional repressive chromatin marks.

13. The method according to claim 4, wherein said cancer is a breast cancer.

14. A method for treating a cancer in a subject in need thereof, comprising administering to the subject a combined preparation comprising a cancer drug and a compound that modulates the epigenetic status of the at least one biomarker according to claim 4.

15. A method for treating cancer in a subject in need thereof, comprising
administering to the subject a therapeutically effective amount of a combined preparation comprising a cancer drug and a compound that modulates the epigenetic status of a biomarker, wherein the biomarker comprises one or more genomic sequence(s) comprising a histone modification, wherein said one or more genomic sequence(s) is/are selected from the list of Tables 1 to 6, and wherein the therapeutically effective amount is sufficient to reduce the development of resistance to said cancer.

16. The method according to claim 5, wherein the method is performed prior to administration of the treatment to the patient.

17. The method according to claim 5, wherein the cancer drug agent is capecitabine.

18. The method according to claim 5, wherein the cancer drug agent is an anti-hormonal drug and said one or more genomic sequence(s) is/are selected from the list of Table 1.

19. The method according to claim 18, wherein the anti-hormonal drug is tamoxifen.

20. The method according to claim 5, wherein the histone modification is associated with transcriptional activation.

21. The method according to claim 20 wherein said histone modification is a loss of H3K23me6 transcriptional repressive chromatin mark.

22. The method according to claim 5 wherein said cancer is a breast cancer.

23. The method according to claim 22, wherein said breast cancer is triple negative breast cancer.

24. The method of claim 15, wherein said compound is administered before, after or concurrently with the therapeutic drug.

25. The method according to claim 4, wherein the method is performed prior to administration of the treatment to the patient.

26. The method according to claim 5, wherein the cancer drug is a chemotherapy drug, and said one or more genomic sequence(s) are selected from the genomic sequences listed in Table 1.
